(19) United States
(12) Patent Application Publication
(10) Pub. No.: US 2005/0267120 A1
(43) Pub. Date: Dec. 1, 2005

(54) Alkyne compounds with MCH antagonistic activity and medicaments comprising these compounds

(75) Inventors: Dirk Stenkamp, Biberach (DE); Stephan Georg Mueller, Warthausen (DE); Philipp Lustenberger, Warthausen (DE); Thorsten Lehmann-Lintz, Ochsenhausen (DE); Gerald Juergen Roth, Biberach (DE); Klaus Rudolf, Warthausen (DE); Marcus Schindler, Biberach (DE); Leo Thomas, Biberach (DE); Ralf Lotz, Schemmerhofen (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 11/104,832

(22) Filed: Apr. 13, 2005

Related U.S. Application Data
(60) Provisional application No. 60/563,654, filed on Apr. 20, 2004.

(30) Foreign Application Priority Data
Apr. 14, 2004 (DE) 10 2004 017 933.6

(51) Int. Cl. A61K 31/501; A61K 31/4709; A61K 31/353

(52) U.S. Cl. 514/252.04; 514/311; 514/312; 514/456; 514/307

(57) ABSTRACT

Alkyne compounds of formula I 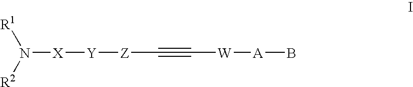
wherein A, B, W, X, Y, Z, R¹, and R² have the meanings given herein, which have MCH-receptor antagonistic activity and are useful for preparing pharmaceutical compositions for the treatment of metabolic disorders and/or eating disorders, particularly obesity and diabetes.

### RELATED APPLICATIONS

This application claims benefit of U.S. Ser. No. 60/563,654, filed Apr. 20, 2004, and claims priority to German Application No. 10 2004 017 933.6, filed Apr. 14, 2004, each of which is hereby incorporated by reference in its entirety.

### FIELD OF THE INVENTION

The present invention relates to new alkyne compounds, the physiologically acceptable salts thereof as well as their use as MCH antagonists and their use in preparing a pharmaceutical preparation which is suitable for the prevention and/or treatment of symptoms and/or diseases caused by MCH or causally connected with MCH in some other way. The invention also relates to the use of a compound according to the invention for influencing eating behavior and for reducing body weight and/or for preventing any increase in body weight in a mammal. It further relates to compositions and medicaments containing a compound according to the invention and processes for preparing them. Other aspects of this invention relate to processes for preparing the compounds according to the invention.

### BACKGROUND OF THE INVENTION

The intake of food and its conversion in the body is an essential part of life for all living creatures. Therefore, deviations in the intake and conversion of food generally lead to problems and also illness. The changes in the lifestyle and nutrition of humans, particularly in industrialized countries, have promoted morbid overweight (also known as corpulence or obesity) in recent decades. In affected people, obesity leads directly to restricted mobility and a reduction in the quality of life. There is the additional factor that obesity often leads to other diseases such as, for example, diabetes, dyslipidemia, high blood pressure, arteriosclerosis, and coronary heart disease. Moreover, high bodyweight alone puts an increased strain on the support and mobility apparatus, which can lead to chronic pain and diseases such as arthritis or osteoarthritis. Thus, obesity is a serious health problem for society.

The term obesity means an excess of adipose tissue in the body. In this connection, obesity is fundamentally to be seen as the increased level of fatness which leads to a health risk. There is no sharp distinction between normal individuals and those suffering from obesity, but the health risk accompanying obesity is presumed to rise continuously as the level of fatness increases. For simplicity's sake, in the present invention, individuals with a Body Mass Index (BMI), which is defined as the bodyweight measured in kilograms divided by the height (in meters) squared, above a value of 25 and more particularly above 30, are preferably regarded as suffering from obesity.

Apart from physical activity and a change in nutrition, there is currently no convincing treatment option for effectively reducing bodyweight. As obesity is a major risk factor in the development of serious and even life-threatening diseases, however, it is all the more important to have access to pharmaceutical active substances for the prevention and/or treatment of obesity. One approach which has been proposed very recently is the therapeutic use of MCH antagonists (cf. inter alia WO 01/21577 and WO 01/82925).

Melanin-concentrating hormone (MCH) is a cyclic neuropeptide consisting of 19 amino acids. It is synthesized predominantly in the hypothalamus in mammals and from there travels to other parts of the brain by the projections of hypothalamic neurons. Its biological activity is mediated in humans through two different glycoprotein-coupled receptors (GPCRs) from the family of rhodopsin-related GPCRs, namely the MCH receptors 1 and 2 (MCH-1R, MCH-2R).

Investigations into the function of MCH in animal models have provided good indications for a role of the peptide in regulating the energy balance, i.e., changing metabolic activity and food intake. D. Qu, et al., A role for melanin-concentrating hormone in the central regulation of feeding behavior, Nature, 1996, 380(6571): pp. 243-7; M. Shimada, et al., Mice lacking melanin-concentrating hormone are hypophagic and lean, Nature, 1998, 396(6712): pp. 670-4. For example, after intraventricular administration of MCH in rats, food intake was increased compared with control animals. Additionally, transgenic rats which produce more MCH than control animals, when given a high-fat diet, responded by gaining significantly more weight than animals without an experimentally altered MCH level. It was also found that there is a positive correlation between phases of increased desire for food and the quantity of MCH mRNA in the hypothalamus of rats. However, experiments with MCH knock-out mice are particularly important in showing the function of MCH. Loss of the neuropeptide results in lean animals with a reduced fat mass, which take in significantly less food than control animals.

The anorectic effects of MCH are presumably mediated in rodents through the G-Galpha i-coupled MCH-1R [B. Borowsky, et al., Antidepressant, anxiolytic and anorectic effects of a melanin-concentrating hormone-1 receptor antagonist, Nat Med, 2002, 8(8): pp. 825-30; Y. Chen, et al., Targeted disruption of the melanin-concentrating hormone receptor-1 results in hyperphagia and resistance to diet-induced obesity, Endocrinology, 2002, 143(7): pp. 2469-77; D. J. Marsh, et al., Melanin-concentrating hormone 1 receptor-deficient mice are lean, hyperactive, and hyperphagic and have altered metabolism. Proc Natl Acad Sci U S A, 2002, 99(5): pp. 3240-5; S. Takekawa, et al., T-226296: A novel, orally active and selective melanin-concentrating hormone receptor antagonist, Eur J Pharmacol, 2002, 438(3): pp. 129-35.], as, unlike primates, ferrets, and dogs, no second MCH receptor subtype has hitherto been found in rodents. After losing the MCH-1R, knock-out mice have a lower fat mass, an increased energy conversion and, when fed on a high fat diet, do not put on weight, compared with control animals. Another indication of the importance of the MCH system in regulating the energy balance results from experiments with a receptor antagonist (SNAP-7941). B. Borowsky, et al., Nat Med, 2002, 8(8): pp. 825-30. In long term trials, the animals treated with the antagonist lose significant amounts of weight.

In addition to its anorectic effect, the MCH-1R antagonist SNAP-7941 also achieves additional anxiolytic and antidepressant effects in behavioral experiments on rats. B. Borowsky, et al., Nat Med, 2002, 8(8): pp. 825-30. Thus, there are clear indications that the MCH-MCH-1R system is involved not only in regulating the energy balance but also in affectivity.

In the patent literature certain amine compounds are proposed as MCH antagonists. Thus, WO 01/21577 (Takeda) describes compounds of formula 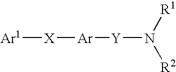
wherein Ar¹ denotes a cyclic group, X denotes a spacer, Y denotes a bond or a spacer, Ar denotes an aromatic ring which may be fused with a non-aromatic ring, R¹ and R² independently of one another denote H or a hydrocarbon group, while R¹ and R² together with the adjacent N atom may form an N-containing hetero ring and R² with Ar may also form a spirocyclic ring, and R together with the adjacent N atom and Y may form an N-containing hetero ring, as MCH antagonists for the treatment of obesity.

Moreover WO 01/82925 (Takeda) also describes compounds of formula 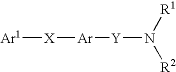
wherein Ar¹ denotes a cyclic group, X and Y represent spacer groups, Ar denotes an optionally substituted fused polycyclic aromatic ring, R¹ and R² independently of one another represent H or a hydrocarbon group, while R¹ and R² together with the adjacent N atom may form an N-containing heterocyclic ring and R² together with the adjacent N atom and Y may form an N-containing hetero ring, as MCH antagonists for the treatment of obesity, inter alia.

WO 2004/024702 proposes carboxylic acid amide compounds of formula I 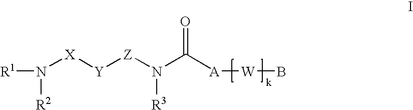
wherein Y, A, and B may represent cyclic groups and X, Z, and W may denote bridges or bonds, as MCH-antagonists.

WO 04/039780 A1 describes alkyne compounds of formula I 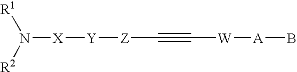
wherein Y, A, and B may denote cyclic groups and X, Z, and W may denote bridges or bonds, as MCH-antagonists. The following substances are mentioned, inter alia: 6-[5-(4-chlorophenyl)pyridin-2-ylethynyl]-2-pyrrolidin-1-ylmethyl-1,2,3,4-tetrahydroquinoline, 6-[5-(4-chlorophenyl)pyridin-2-ylethynyl]-1-methyl-2-pyrrolidin-1-ylmethyl-1,2,3,4-tetrahydroquinoline, and 5-(4-chlorophenyl)-2-[2-(4-methylpiperidin-1-ylmethyl)chroman-6-ylethynyl]pyridine.

WO 04/039764 A1 describes amide compounds of formula I 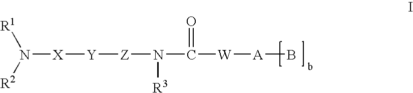
wherein Y, A, and B may denote cyclic groups and X denotes an alkylene bridge, Z denotes a bridge or bond and W is selected from the group comprising —CR^{6a}R^{6b}—O, —CR^{7a}═CR^{7c}, —CR^{6a}R^{6b}—NR⁸, —CR^{7a}R^{7b}—CR^{7c}R^{7d}, and —NR⁸—CR^{6a}R^{6b}—, as MCH-antagonists.

The aim of the present invention is to identify new alkyne compounds, particularly those which are especially effective as MCH antagonists. The invention also sets out to provide new alkyne compounds which can be used to influence the eating habits of mammals and achieve a reduction in body weight, particularly in mammals, and/or prevent an increase in body weight.

The present invention further sets out to provide new pharmaceutical compositions which are suitable for the prevention and/or treatment of symptoms and/or diseases caused by MCH or otherwise causally connected to MCH. In particular, the aim of this invention is to provide pharmaceutical compositions for the treatment of metabolic disorders such as obesity and/or diabetes as well as diseases and/or disorders which are associated with obesity and diabetes. Other objectives of the present invention are concerned with demonstrating advantageous uses of the compounds according to the invention. The invention also sets out to provide a process for preparing the amide compounds according to the invention. Other aims of the present invention will be immediately apparent to the skilled man from the foregoing remarks and those that follow.

In a first aspect, the present invention-relates to alkyne compounds of general formula I 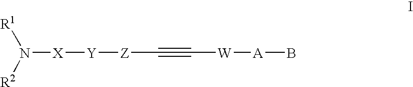
wherein:
R¹ and R² independently of one another denote H, C₁₋₈-alkyl, C₃₋₇-cycloalkyl, or a phenyl or pyridinyl group optionally mono- or polysubstituted by identical or different groups R²⁰ and/or monosubstituted by nitro, while the alkyl or cycloalkyl group may be mono- or polysubstituted by identical or different groups R¹¹, and a —CH₂— group in position 3 or 4 of a 5-, 6-, or 7-membered cycloalkyl group may be replaced by —O—, —S—, or —NR¹³—, or
R¹ and R² form a C₃₋₈-alkylene bridge, wherein a —CH₂— group not adjacent to the N atom of the R¹R²N— group may be replaced by —CH═N—, —CH═CH—, —O—, —S—, —SO—, —(SO₂)—, —CO—, —C(═CH₂)—, or —NR¹³—, while in the alkylene bridge defined hereinbefore one or more H atoms may be replaced by identical or different groups R¹⁴, and the alkylene bridge defined hereinbefore may be substituted by one or two identical or different carbo- or heterocyclic groups Cy such that the bond between the alkylene bridge and the group Cy is made via a single or double bond, via a common C atom forming a spirocyclic ring system, via two common adjacent C and/or N atoms forming a fused bicyclic ring system, or via three or more C and/or N atoms forming a bridged ring system;
X denotes a C₁₋₄-alkylene bridge, while in the definition C₂₋₄-alkylene one or two C atoms may be monosubstituted by R¹⁰, or a C₃₋₄-alkylene bridge, wherein a —CH₂—CH₂— group not immediately adjacent to the N atom of the R¹R²N— group is replaced by —CH═CH— or —C≡C—, while the meanings given above for X may include a substituent selected from C₂₋₆-alkenyl, C₂₋₆-alkynyl, C₃₋₇-cycloalkyl, and C₃₋₇-cycloalkyl-C₁₋₃-alkyl as well as, independently, one, two, or three identical or different C₁₋₄-alkyl substituents, while two alkyl groups may be joined together forming a 3- to 7-membered cyclic group or an alkyl and an alkenyl group may be joined together forming a 5- to 7-membered cyclic group, and
W and Z independently of one another denote a single bond or a C₁₋₂-alkylene bridge, while two adjacent C atoms may be joined together by an additional C₁₋₄-alkylene bridge, and one or two C atoms independently of one another may be substituted by one or two identical or different C₁₋₃-alkyl groups, while two alkyl groups may be joined together, forming a carbocyclic ring, and
Y is selected from the definitions of the partial formulae Y1 and Y2 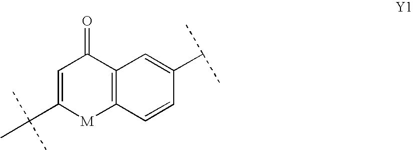 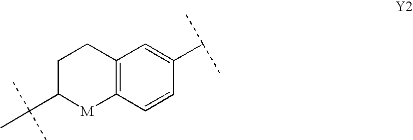
   wherein the group M denotes O, S, or NR^{M}, where R^{M} has a meaning selected from among H, C₁₋₆-alkyl, C₃₋₆-alkenyl, C₃₋₆-alkynyl, C₃₋₇-cycloalkyl, and C₃₋₇-cycloalkyl-C₁₋₃-alkyl, and
   in the partial formulae Y1 and Y2, one or more C atoms may be substituted independently of one another by R²⁰; and
A is selected from among the bivalent cyclic groups phenyl, pyridinyl, pyrimidinyl, pyrazinyl, pyridazinyl, naphthyl, tetrahydronaphthyl, indolyl, dihydroindolyl, quinolinyl, dihydroquinolinyl, tetrahydroquinolinyl, isoquinolinyl, dihydroisoquinolinyl, tetrahydroisoquinolinyl, benzimidazolyl, benzoxazolyl, thienyl, furanyl, benzothienyl, or benzofuranyl, while the abovementioned cyclic groups may be mono- or polysubstituted at one or more C atoms by identical or different groups R²⁰, and in the case of a phenyl ring may also additionally be monosubstituted by nitro, and/or one or more NH groups may be substituted by R²¹;
B has one of the meanings given for A or denotes C₁₋₆-alkyl, C₁₋₆-alkenyl, C₁₋₆-alkynyl, C₃₋₇-cycloalkyl, C₅₋₇-cycloalkenyl, C₃₋₇-cycloalkyl-C₁₋₃-alkyl, C₃₋₇-cycloalkenyl-C₁₋₃-alkyl, C₃₋₇cycloalkyl-C₁₋₃-alkenyl, or C₃₋₇-cycloalkyl-C₁₋₃-alkynyl, wherein one or more C atoms independently of one another may be mono- or polysubstituted by halogen and/or monosubstituted by hydroxy or cyano and/or cyclic groups may be mono- or polysubstituted by identical or different groups R²⁰;
Cy denotes a carbo- or heterocyclic group selected from one of the following meanings: a saturated 3- to 7-membered carbocyclic group, an unsaturated 4- to 7-membered carbocyclic group, a phenyl group, a saturated 4- to 7-membered or unsaturated 5- to 7-membered heterocyclic group with an N, O, or S atom as heteroatom, a saturated or unsaturated 5- to 7-membered heterocyclic group with two or more N atoms or with one or two N atoms and an O or S atom as heteroatoms, or an aromatic heterocyclic 5- or 6-membered group with one or more identical or different heteroatoms selected from N, O, and/or S,
   while the saturated 6- or 7-membered groups mentioned above may also be present as bridged ring systems with an imino, (C₁₋₄-alkyl)-imino, methylene, (C₁₋₄-alkyl)-methylene or di-(C₁₋₄-alkyl)-methylene bridge, and
   the abovementioned cyclic groups may be mono- or polysubstituted at one or more C atoms by identical or different groups R²⁰, and in the case of a phenyl group may also additionally be monosubstituted by nitro, and/or one or more NH groups may be substituted by R²¹;
R¹⁰ denotes hydroxy, ω-hydroxy-C₁₋₃-alkyl, C₁₋₄-alkoxy, or C₁₋₄-alkoxy-C₁₋₃-alkyl;
R¹¹ denotes halogen, C₁₋₆-alkyl, C₂₋₆-alkenyl, C₂₋₆-alkynyl, R¹⁵—O—, R¹⁵—O—CO—, R¹⁵—CO—O— cyano, R¹⁶R¹⁷N, R¹⁸R¹⁹N—CO—, or Cy, while in the abovementioned groups one or more C atoms may be substituted independently of one another by substituents selected from halogen, OH, CN, CF₃, C₁₋₃-alkyl, and hydroxy-C₁₋₃-alkyl;
R¹³ has one of the meanings given for R¹⁷;
R¹⁴ denotes halogen, cyano, C₁₋₆-alkyl, C₂₋₆-alkenyl, C₂₋₆-alkynyl, R¹⁵—O—R¹⁵—O—CO—,R¹⁵—CO—, R¹⁵—CO—O—R¹⁶R¹⁷N, R¹⁸R¹⁹N—CO—,R¹⁵—O—C₁₋₃-alkyl, R¹⁵—O—CO—C₁₋₃-alkyl, R¹⁵—SO₂—NH—, R¹⁵—O—CO—NH—C₁₋₃-alkyl, R¹⁵—SO₂—NH—C₁₋₃-alkyl, R¹⁵—CO—C₁₋₃-alkyl, R¹⁵—CO—O—C₁₋₃-alkyl, R¹⁶R¹⁷N—C₁₋₃-alkyl, R¹⁸R¹⁹N—CO—C₁₋₃-alkyl, or Cy-C₁₋₃-alkyl;
R¹⁵ denotes H, C₁₋₄-alkyl, C₃₋₇-cycloalkyl, C₃₋₇-cycloalkyl-C₁₋₃-alkyl, phenyl, phenyl-C₁₋₃-alkyl, pyridinyl, or pyridinyl-C₁₋₃-alkyl;
R¹⁶ denotes H, C₁₋₆-alkyl, C₃₋₇-cycloalkyl, C₃₋₇-cycloalkyl-C₁₋₃-alkyl, C₄₋₇-cycloalkenyl, C₄₋₇-cycloalkenyl-C₁₋₃-alkyl, ω-hydroxy-C₂₋₃-alkyl, ω-(C₁₋₄-alkoxy)-C₂₋₃-alkyl, amino-C₂₋₆-alkyl, C₁₋₄-alkyl-amino-C₂₋₆-alkyl, di-(C₁₋₄-alkyl)-amino-C₂₋₆-alkyl, or cyclo-C₃₋₆-alkyleneimino-C₂₋₆-alkyl;
R¹⁷ has one of the meanings given for R¹⁶ or denotes phenyl, phenyl-C₁₋₃-alkyl, pyridinyl, C₁₋₄-alkylcarbonyl, hydroxycarbonyl-C₁₋₃-alkyl, C₁₋₄-alkoxycarbonyl, C₁₋₄-alkoxycarbonyl-C₁₋₃-alkyl, C₁₋₄-alkylcarbonylamino-C₂₋₃-alkyl, N-(C₁₋₄-alkylcarbonyl)-N-(C₁₋₄-alkyl)-amino-C₂₋₃-alkyl, C₁₋₄-alkylsulfonyl, C₁₋₄-alkylsulfonylamino-C₂₋₃-alkyl, or N-(C₁₋₄-alkylsulfonyl)-N(—C₁₋₄-alkyl)-amino-C₂₋₃-alkyl;
R¹⁸ and R¹⁹ independently of one another denote H or C₁₋₆-alkyl,
R²⁰ denotes halogen, hydroxy, cyano, C₁₋₆-alkyl, C₂₋₆-alkenyl, C₂₋₆-alkynyl, C₃₋₇-cycloalkyl, C₃₋₇-cycloalkyl-C₁₋₃-alkyl, hydroxy-C₁₋₃-alkyl, R²²—C₁₋₃-alkyl, or has one of the meanings given for R²²;
R²¹ denotes C₁₋₄-alkyl, ω-hydroxy-C₂₋₆-alkyl, ω-C₁₋₄-alkoxy-C₂₋₆-alkyl, ω-C₁₋₄-alkyl-amino-C₂₋₆-alkyl, ω-di-(C₁₋₄-alkyl)-amino-C₂₋₆-alkyl, ω-cyclo-C₃₋₆-alkyleneimino-C₂₋₆-alkyl, phenyl, phenyl-C₁₋₃-alkyl, C₁₋₄-alkyl-carbonyl, C₁₋₄-alkoxy-carbonyl, C₁₋₄-alkylsulfonyl, aminosulfonyl, C₁₋₄-alkylaminosulfonyl, di-C₁₋₄-alkylaminosulfonyl, or cyclo-C₃₋₆-alkylene-iminosulfonyl;
R²² denotes pyridinyl, phenyl, phenyl-C₁₋₃-alkoxy, cyclo-C₃₋₆-alkyleneimino-C₂₋₄-alkoxy, OHC—, HO—N═HC—, C₁₋₄-alkoxy-N═HC—, C₁₋₄-alkoxy, C₁₋₄-alkylthio, carboxy, C₁₋₄-alkylcarbonyl, C₁₋₄-alkoxycarbonyl, aminocarbonyl, C₁₋₄-alkylaminocarbonyl, di-(C₁₋₄-alkyl)-aminocarbonyl, cyclo-C₃₋₆-alkyl-aminocarbonyl, cyclo-C₃₋₆-alkyleneiminocarbonyl, phenylaminocarbonyl, cyclo-C₃₋₆-alkyleneimino-C₂₋₄-alkyl-aminocarbonyl, C₁₋₄-alkyl-sulfonyl, C₁₋₄-alkyl-sulfinyl, C₁₋₄-alkyl-sulfonylamino, amino, C₁₋₄-alkylamino, di-(C₁₋₄-alkyl)-amino, C₁₋₄-alkyl-carbonylamino, cyclo-C₃₋₆-alkyleneimino, phenyl-C₁₋₃-alkylamino, N-(C₁₋₄-alkyl)-phenyl-C₁₋₃-alkylamino, acetylamino, propionylamino, phenylcarbonyl, phenylcarbonylamino, phenylcarbonylmethylamino, hydroxy-C₂₋₃-alkylaminocarbonyl, (4-morpholinyl)carbonyl, (1-pyrrolidinyl)carbonyl, (1-piperidinyl)carbonyl, (hexahydro-1-azepinyl)carbonyl, (4-methyl-1-piperazinyl)carbonyl, methylenedioxy, aminocarbonylamino, or C₁₋₄-alkylaminocarbonylamino, while in the abovementioned groups and residues, particularly in W, X, Z, and R¹³ to R²², in each case one or more C atoms may additionally be mono- or polysubstituted by F and/or in each case one or two C atoms independently of one another may additionally be monosubstituted by Cl or Br and/or in each case one or more phenyl rings may independently of one another additionally contain one, two or three substituents selected from among F, Cl, Br, I, cyano, C₁₋₄-alkyl, C₁₋₄-alkoxy, difluoromethyl, trifluoromethyl, hydroxy, amino, C₁₋₃-alkylamino, di-(C₁₋₃-alkyl)-amino, acetylamino, aminocarbonyl, difluoromethoxy, trifluoromethoxy, amino-C₁₋₃-alkyl, C₁₋₃-alkylamino-C₁₋₃-alkyl-, and di-(C₁₋₃-alkyl)-amino-C₁₋₃-alkyl, and/or may be monosubstituted by nitro, and
the H atom of any carboxy group present or an H atom bound to an N atom may each be replaced by a group which can be cleaved in vivo,
the tautomers, the diastereomers, the enantiomers, the mixtures thereof and the salts thereof,
while the following compounds are not included according to the invention:
6-[5-(4-chlorophenyl)pyridin-2-ylethynyl]-2-pyrrolidin-1-ylmethyl-1,2,3,4-tetrahydroquinoline,
6-[5-(4-chlorophenyl)pyridin-2-ylethynyl]-1-methyl-2-pyrrolidin-1-ylmethyl-1,2,3,4-tetrahydroquinoline; and
5-(4-chlorophenyl)-2-[2-(4-methylpiperidin-1-ylmethyl)chroman-6-ylethynyl]pyridine.

The compounds according to the present invention, including the physiologically acceptable salts, are especially effective, compared with known, structurally similar compounds, as antagonists of the MCH receptor, particularly the MCH-1 receptor, and exhibit very good affinity in MCH receptor binding studies. In addition, the compounds according to the invention have a high to very high selectivity with regard to the MCH receptor. Generally the compounds according to the invention have low toxicity, they are well absorbed by oral route and have good intracerebral transitivity, particularly brain accessibility.

The invention also relates to the compounds in the form of the individual optical isomers, mixtures of the individual enantiomers or racemates, in the form of the tautomers and in the form of the free bases or corresponding acid addition salts with pharmacologically acceptable acids. The subject of the invention also includes the compounds according to the invention, including their salts, wherein one or more hydrogen atoms are replaced by deuterium.

This invention also includes the physiologically acceptable salts of the alkyne compounds according to the invention as described above and hereinafter.

Also covered by this invention are compositions containing at least one alkyne compound according to the invention and/ or a salt according to the invention optionally together with one or more physiologically acceptable excipients.

Also covered by this invention are pharmaceutical compositions containing at least one alkyne compound according to the invention and/ or a salt according to the invention optionally together with one or more inert carriers and/or diluents.

This invention also relates to the use of at least one alkyne compound according to the invention and/or a salt according to the invention for influencing the eating behavior of a mammal.

The invention further relates to the use of at least one alkyne compound according to the invention and/or a salt according to the invention for reducing the body weight and/ or for preventing an increase in the body weight of a mammal.

The invention also relates to the use of at least one alkyne compound according to the invention and/or a salt according to the invention for preparing a pharmaceutical composition with an MCH receptor-antagonistic activity, particularly with an MCH-1 receptor-antagonistic activity.

This invention also relates to the use of at least one alkyne compound according to the invention and/or a salt according to the invention for preparing a pharmaceutical composition which is suitable for the prevention and/or treatment of symptoms and/or diseases which are caused by MCH or are otherwise causally connected with MCH.

A further object of this invention is the use of at least one alkyne compound according to the invention and/or a salt according to the invention for preparing a pharmaceutical composition which is suitable for the prevention and/or treatment of metabolic disorders and/or eating disorders, particularly obesity, bulimia, bulimia nervosa, cachexia, anorexia, anorexia nervosa and hyperphagia.

The invention also relates to the use of at least one alkyne compound according to the invention and/or a salt according to the invention for preparing a pharmaceutical composition which is suitable for the prevention and/or treatment of diseases and/or disorders associated with obesity, particularly diabetes, especially type II diabetes, complications of diabetes including diabetic retinopathy, diabetic neuropathy, diabetic nephropathy, insulin resistance, pathological glucose tolerance, encephalorrhagia, cardiac insufficiency, cardiovascular diseases, particularly arteriosclerosis and high blood pressure, arthritis and gonitis.

In addition the present invention relates to the use of at least one alkyne compound according to the invention and/or a salt according to the invention for preparing a pharmaceutical composition which is suitable for the prevention and/or treatment of hyperlipidemia, cellulitis, fat accumulation, malignant mastocytosis, systemic mastocytosis, emotional disorders, affective disorders, depression, anxiety, sleep disorders, reproductive disorders, sexual disorders, memory disorders, epilepsy, forms of dementia and hormonal disorders.

The invention also relates to the use of at least one alkyne compound according to the invention and/or a salt according to the invention for preparing a pharmaceutical composition which is suitable for the prevention and/or treatment of urinary problems, such as, for example, urinary incontinence, overactive bladder, urgency, nycturia and enuresis.

The invention further relates to the use of at least one alkyne compound according to the invention and/ or a salt according to the invention for preparing a pharmaceutical composition which is suitable for the prevention and/or treatment of dependencies and/or withdrawal symptoms.

The invention further relates to processes for preparing for preparing a pharmaceutical composition according to the invention, characterized in that at least one alkyne compound according to the invention and/or a salt according to the invention is incorporated in one or more inert carriers and/or diluents by a non-chemical method.

The invention also relates to a pharmaceutical composition containing a first active substance which is selected from the alkyne compounds according to the invention and/or the corresponding salts as well as a second active substance which is selected from the group consisting of active substances for the treatment of diabetes, active substances for the treatment of diabetic complications, active substances for the treatment of obesity, preferably other than MCH antagonists, active substances for the treatment of high blood pressure, active substances for the treatment of dyslipidemia or hyperlipidemia, including arteriosclerosis, active substances for the treatment of arthritis, active substances for the treatment of anxiety states and active substances for the treatment of depression, optionally together with one or more inert carriers and/or diluents.

Moreover, in one aspect, the invention relates to a process for preparing alkyne compounds of formula A.5
R¹R²N—X—Y—C≡C—W—A—B (A.5)
while in formulae A.1, A.2, A.3, A.4, and A.5 R¹, R², Y, X, W, A, and B have one of the meanings given hereinbefore and hereinafter,
wherein a halogen compound of formula A.1
   HO—X—Y—Hal (A.1)
   wherein Hal denotes chlorine, bromine or iodine, preferably bromine or iodine, is reacted with an alkyne compound of formula A.2
   H—C≡C—W-A-B (A.2)
   in the presence of a suitable palladium catalyst, a suitable base and copper (I) iodide in a suitable solvent, and
the compound of formula A.3 obtained
   HO—X—Y—C≡C—W-A-B (A.3)
   is reacted with methanesulfonic acid chloride (MsCI) to produce the methanesulfonate derivative A.4,
   MsO—X—Y—C≡C—W-A-B (A.4)
   which is further reacted with an amine of formula H—NR¹R² to form the end product A.5.

This invention further relates to a process for preparing alkyne compounds of formula B.5
R¹R²N—X—Y-Z-C≡C-A-B (B.5)
while in formulae B.1, B.2, B.3, B.4, and B.5, R¹, R², X, Y, Z, A, and B have one of the meanings given hereinbefore and hereinafter,
wherein a halogen compound of formula B.1
Hal-A-B (B.1)
wherein Hal denotes chlorine, bromine, or iodine, preferably bromine or iodine, is reacted with an alkyne compound of formula B.2
HO—X—Y-Z-C≡C—H (B.2)
in the presence of a suitable palladium catalyst, a suitable base, and copper (I) iodide in a suitable solvent, and
the resulting compound of formula B.3
   HO—X—Y-Z-C≡C-A-B (B.3)
   is reacted with methanesulfonic acid chloride (MsCl) to form the methanesulfonate derivative B.4,
   MsO—X—Y-Z-C≡C-A-B (B.4)
   which is reacted further with an amine of formula H—NR¹R² to form the end product B.5.

In addition, the invention relates to a process for preparing alkyne compounds of formula C.3
R¹R²N—X—Y—C≡C—W-A-B (C.3)
while in formulae C.1, C.2, and C.3, R¹, R², X, Y, W, A, and B have one of the meanings given hereinbefore and hereinafter,
wherein a halogen compound of formula C.1
   R¹R²N—X—Y-Hal (C.1)
   wherein Hal denotes chlorine, bromine, or iodine, preferably bromine or iodine, is further reacted with an alkyne compound of formula C.2
   H—C≡C—W-A-B (C.2)
   in the presence of a suitable palladium catalyst, a suitable base, and copper (I) iodide in a suitable solvent to yield the end product C.3.

In another aspect the invention relates to a process for preparing alkyne compounds of formula D.3
R¹R²N—X—Y-Z-C≡C-A-B (D.3)
while in formulae D.1, D.2, and D.3, R¹, R², X, Y, Z, A, and B have one of the meanings given hereinbefore and hereinafter, wherein a halogen compound of formula D.2
Hal-A-B (D.2)
wherein Hal denotes chlorine, bromine, or iodine, preferably bromine or iodine, is reacted with an alkyne compound of formula D.1
R¹R²N—X—Y-Z-C≡C—H (D.1)
in the presence of a suitable palladium catalyst, a suitable base and copper (I) iodide in a suitable solvent to form the end product D.3.

The starting materials and intermediate products used in the synthesis according to the invention are also a subject of this invention.

### DETAILED DESCRIPTION OF THE INVENTION

Unless otherwise specified, the groups, residues and substituents, particularly A, B, W, X, Y, Z, Cy, R¹, R¹, R², R¹⁰, R¹¹, R¹³ to R²², and R^{M}, have the meanings given hereinbefore.

If groups, residues and/or substituents occur more than once in a compound, they may have the same or different meanings in each case.

If R¹ and R² are not joined together via an alkylene bridge, R¹ and R² independently of one another preferably denote a C₁₋₈-alkyl or C₃₋₇-cycloalkyl group mono- or polysubstituted by identical or different groups R¹¹, while a —CH₂— group in position 3 or 4 of a 5-, 6-, or 7-membered cycloalkyl group may be replaced by —O—, —S—, or —NR¹³—, or a phenyl or pyridinyl group optionally mono- or polysubstituted by identical or different groups R²⁰ and/or monosubstituted by nitro, while one or both of the groups R¹ and R² may also represent H.

Preferred meanings of the group R¹¹ are F, Cl, Br, C₁₋₆-alkyl, C₂₋₆-alkenyl, C₂₋₆-alkynyl, R¹⁵—O-cyano, R¹⁶R¹⁷N, C₃₋₇-cycloalkyl, cyclo-C₃₋₆-alkyleneimino, pyrrolidinyl, N—(C₁₋₄-alkyl)-pyrrolidinyl, piperidinyl, N—(C₁₋₄-alkyl)-piperidinyl, phenyl, and pyridyl, while in the abovementioned groups and radicals one or more C atoms may be mono- or polysubstituted independently of one another by F, C₁₋₃-alkyl, or hydroxy-C₁₋₃-alkyl, and/or one or two C atoms may be monosubstituted independently of one another by Cl, Br, OH, CF₃, or CN, and the abovementioned cyclic groups may be mono- or polysubstituted at one or more C atoms by identical or different radicals R²⁰, or in the case of a phenyl group may also additionally be monosubstituted by nitro, and/or one or more NH groups may be substituted by R²¹. If R¹¹ has one of the meanings R¹⁵—O-cyano, R¹⁶R¹⁷N, or cyclo-C₃₋₆-alkyleneimino, the C atom of the alkyl or cycloalkyl group substituted by R¹¹ is preferably not directly connected to a heteroatom, such as, for example, the group —N—X—.

Preferably the groups R¹, R² independently of one another represent H, C₁₋₆-alkyl, C₃₋₅-alkenyl, C₃₋₅-alkynyl, C₃₋₇-cycloalkyl, hydroxy-C₃₋₇-cycloalkyl, C₃₋₇-cycloalkyl-C₁₋₃-alkyl, (hydroxy-C₃₋₇-cycloalkyl)-C₁₋₃-alkyl, hydroxy-C₂₋₄-alkyl, ω-NC—C₂₋₃-alkyl, C₁₋₄-alkoxy-C₂₋₄-alkyl, hydroxy-C₁₋₄-alkoxy-C₂₋₄-alkyl, C₁₋₄-alkoxy-carbonyl-C₁₋₄-alkyl, carboxyl-C₁₋₄-alkyl, amino-C₂₋₄-alkyl, C₁₋₄-alkyl-amino-C₂₋₄-alkyl, di-(C₁₋₄-alkyl)-amino-C₂₋₄-alkyl, cyclo-C₃₋₆-alkyleneimino-C₂₋₄-alkyl, pyrrolidin-3-yl, N—(C₁₋₄-alkyl)-pyrrolidin-3-yl, pyrrolidinyl-C₁₋₃-alkyl, N—(C₁₋₄-alkyl)-pyrrolidinyl-C₁₋₃-alkyl, piperidin-3-yl, piperidin-4-yl, N-(C₁₋₄-alkyl)-piperidin-3-yl, N—(C₁₋₄-alkyl)-piperidin-4-yl, piperidinyl-C₁₋₃-alkyl, N—(C₁₋₄-alkyl)-piperidinyl-C₁₋₃-alkyl, tetrahydropyran-3-yl, tetrahydropyran-4-yl, phenyl, phenyl-C₁₋₃-alkyl, pyridyl, or pyridyl-C₁₋₃-alkyl, while in the abovementioned groups and radicals one or more C atoms independently of one another may be mono- or polysubstituted by F, C₁₋₃-alkyl, or hydroxy-C₁₋₃-alkyl, and/or one or two C atoms independently of one another may be monosubstituted by Cl, Br, OH, CF₃, or CN, and the abovementioned cyclic groups may be mono- or polysubstituted at one or more C atoms by identical or different radicals R²⁰, in the case of a phenyl group may also additionally be monosubstituted by nitro, and/or one or more NH groups may be substituted by R²¹. Preferred substituents of the abovementioned phenyl or pyridyl groups are selected from the group F, Cl, Br, I, cyano, C₁₋₄-alkyl, C₁₋₄-alkoxy, difluoromethyl, trifluoromethyl, hydroxy, amino, C₁₋₃-alkylamino, di-(C₁₋₃-alkyl)-amino, acetylamino, aminocarbonyl, difluoromethoxy, trifluoromethoxy, amino-C₁₋₃-alkyl, C₁₋₃-alkylamino-C₁₋₃-alkyl, and di-(C₁₋₃-alkyl)-amino-C₁₋₃-alkyl, while a phenyl group may also be monosubstituted by nitro. Particularly preferred definitions of the groups R¹ and/or R² are selected from the group consisting of H, C₁₋₄-alkyl, hydroxy-C₁₋₄-alkyl, C₃₋₅-alkenyl, C₃₋₅-alkynyl, C₃₋₇-cycloalkyl, hydroxy-C₃₋₇-cycloalkyl, dihydroxy-C₃₋₄-alkyl, C₃₋₇-cycloalkyl-C₁₋₃-alkyl, tetrahydropyran-3-yl, tetrahydropyran-4-yl, (hydroxy-C₃₋₇-cycloalkyl)-C₁₋₃-alkyl, ω-(C₁₋₄-alkoxy)-C₂₋₃-alkyl, pyridyl, and benzyl, while an alkyl, cycloalkyl or cycloalkyl-alkyl group may additionally be mono- or disubstituted by hydroxy and/or hydroxy-C₁₋₃-alkyl, and/or mono- or polysubstituted by F or C₁₋₃-alkyl and/or monosubstituted by CF₃, Br, Cl, or CN, and one of the groups R¹ and R² may also denote H.

Most particularly preferred groups R¹ and/or R² are selected from the group consisting of H, methyl, ethyl, n-propyl, isopropyl, prop-2-enyl, but-2-enyl, prop-2-ynyl, but-2-ynyl, 2-methoxyethyl, cyclopropyl, cyclopentyl, cyclohexyl, cyclopropylmethyl, cyclopentylmethyl, hydroxy-C₃₋₇-cycloalkyl, (hydroxy-C₁₋₃-alkyl)-hydroxy-C₃₋₇-cycloalkyl, dihydroxy-C₃₋₅-alkyl, 2-hydroxy-1-(hydroxymethyl)ethyl, 1,1-di(hydroxymethyl)ethyl, (1-hydroxy-C₃₋₆-cycloalkyl)methyl, tetrahydropyran-3-yl, tetrahydropyran-4-yl, 2-hydroxyethyl, 3-hydroxypropyl, benzyl, and pyridyl, while the abovementioned groups may be mono- or polysubstituted by F and/or C₁₋₃-alkyl, and the phenyl and pyridyl rings may be substituted as specified.

Examples of most particularly preferred groups R¹ and/or R² are therefore H, methyl, ethyl, n-propyl, isopropyl, prop-2-enyl, prop-2-ynyl, 2-methoxyethyl, cyclopropyl, cyclopentyl, cyclohexyl, cyclopropylmethyl, cyclopentylmethyl, hydroxycyclopentyl, hydroxycyclohexyl, (hydroxymethyl)hydroxycyclopentyl, (hydroxymethyl)hydroxycyclohexyl, 2,3-dihydroxypropyl, (1-hydroxycyclopropyl)methyl, tetrahydropyran-3-yl, tetrahydropyran-4-yl, 2-hydroxyethyl, 3-hydroxypropyl, benzyl, and pyridyl.

Particularly preferably, at least one of the groups R¹, R², and most particularly preferably both groups, have a meaning other than H.

If R¹ and R² form an alkylene bridge, this is preferably a C₃₋₇-alkylene bridge or a C₃₋₇-alkylene bridge, particularly a C₄₋₇-alkylene bridge, wherein a —CH₂— group not adjacent to the N atom of the R¹R²N— group is replaced by —CH═N—, —CH═CH—, —O—, —S—, —CO—, or —NR¹³—,
while in the alkylene bridge defined hereinbefore one or more H atoms may be replaced by identical or different groups R¹⁴, and
the alkylene bridge defined hereinbefore may be substituted with a carbo- or heterocyclic group Cy in such a way that the bond between the alkylene bridge and the group Cy is made via a single or double bond, via a common C atom forming a spirocyclic ring system, via two common adjacent C and/or N atoms forming a fused bicyclic ring system, or via three or more C and/or N atoms forming a bridged ring system.

Preferably also, R¹ and R² form an alkylene bridge such that R¹R²N— denotes a group which is selected from azetidine, pyrrolidine, piperidine, azepan, 2,5-dihydro-1H-pyrrole, 1,2,3,6-tetrahydropyridine, 2,3,4,7-tetrahydro-1H-azepine, 2,3,6,7-tetrahydro-1H-azepine, piperazine in which the free imine function is substituted by R¹³, piperidin-4-one, morpholine, and thiomorpholine, is particularly preferably selected from pyrrolidine, piperidine, piperazine in which the free imine function is substituted by R¹³, and morpholine, while according to the general definition of R¹ and R² one or more H atoms may be replaced by identical or different groups R¹⁴, and/or the abovementioned groups may be substituted by one or two identical or different carbo- or heterocyclic groups Cy in a manner specified according to the general definition of R¹ and R², while the group Cy may be mono- or polysubstituted by R²⁰.

Particularly preferred groups Cy are C₃₋₇-cycloalkyl or aza-C₄₋₇-cycloalkyl, particularly cyclo-C₃₋₆-alkyleneimino, as well as 1-C₁₋₄-alkyl-aza-C₄₋₇-cycloalkyl, while the group Cy may be mono- or polysubstituted by R²⁰.

The C₃₋₈-alkylene bridge formed by R¹ and R², wherein —CH₂— groups may be replaced as specified, may be substituted, as described, by one or two identical or different carbo- or heterocyclic groups Cy, which may be substituted as specified hereinbefore.

In the event that the alkylene bridge is linked to a group Cy through a single bond, Cy is preferably selected from the group consisting of C₃₋₇-cycloalkyl, cyclo-C₃-6-alkyleneimino, 1H-imidazole, thienyl, and phenyl.

In the event that the alkylene bridge is linked to a group Cy via a common C atom forming a spirocyclic ring system, Cy is preferably selected from the group consisting of C₃₋₇-cycloalkyl, aza-C₄₋₈-cycloalkyl, oxa-C₄₋₈-cycloalkyl, and 2,3-dihydro-1H-quinazolin-4-one.

In the event that the alkylene bridge is linked to a group Cy via two common adjacent C and/or N atoms forming a fused bicyclic ring system, Cy is preferably selected from the group consisting of C₄₋₇-cycloalkyl, phenyl, and thienyl.

In the event that the alkylene bridge is linked to a group Cy via three or more C and/or N atoms forming a bridged ring system, Cy preferably denotes C₄₋₈-cycloalkyl or aza-C₄₋₈-cycloalkyl.

In the event that the heterocyclic group R¹R²N— is substituted by a group Cy, the group Cy is preferably linked to the group R¹R²N— through a single bond, while Cy is preferably selected from the group consisting of C₃₋₇-cycloalkyl and cyclo-C₃₋₆-alkyleneimino, while these groups may be substituted as specified, preferably by fluorine, CF₃, C₁₋₃-alkyl, hydroxy-C₁₋₃-alkyl, and hydroxy.

Particularly preferably the group 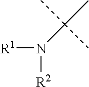
is defined according to one of the following partial formulae 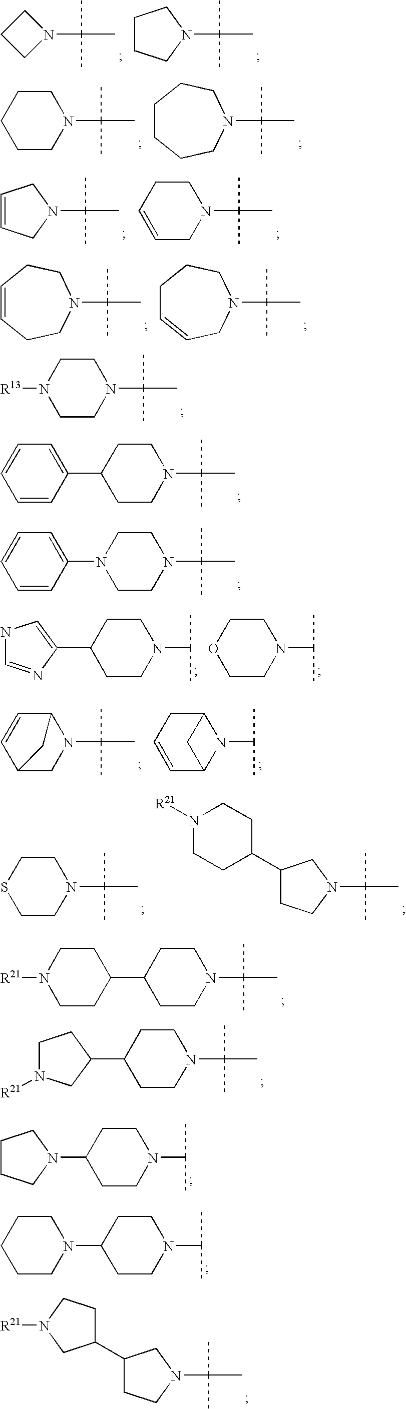 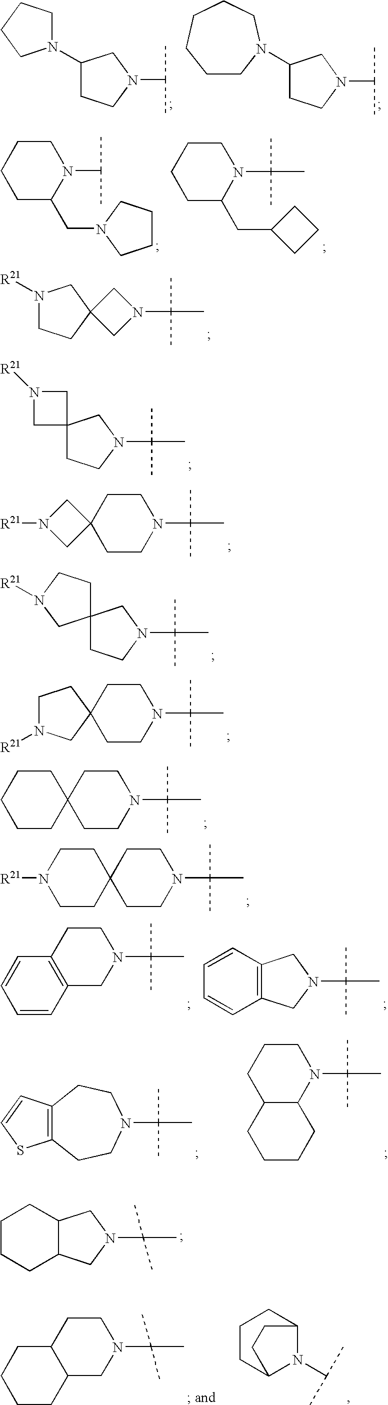
wherein one or more H atoms of the heterocycle formed by the group R¹R²N— may be replaced by identical or different groups R¹⁴, and
the heterocycle formed by the group R¹R²N— may be substituted by one or two, preferably one C₃₋₇-cycloalkyl group, while the cycloalkyl group may be mono- or polysubstituted by R²⁰, and
the ring linked to the heterocycle formed by the group R¹R²N— may be mono- or polysubstituted at one or more C atoms by R²⁰, and in the case of a phenyl ring may also additionally be monosubstituted by nitro and
wherein R¹³, R¹⁴, R²⁰, and R²¹ have the meanings given above and hereinafter.

If the heterocycle formed by the group R¹R²N— is substituted as specified by one or two cycloalkyl groups mono- or polysubstituted by R²⁰, the substituents R²⁰ independently of one another preferably denote C₁₋₄-alkyl, C₁₋₄-alkoxy-C₁₋₃-alkyl, hydroxy-C₁₋₃-alkyl, hydroxy, fluorine, chlorine, bromine, or CF₃, particularly hydroxy.

Most particularly preferably the group 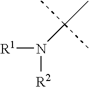
is defined according to one of the following partial formulae 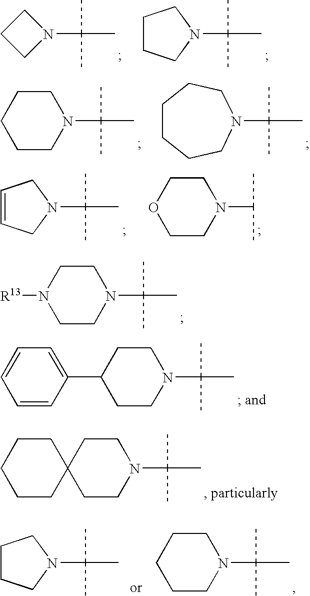
where R¹³ has the meanings given above and hereinafter, and
the heterocycle formed by the group R¹R²N— may be substituted by C₃₋₆-cycloalkyl, hydroxy-C₃₋₆-cycloalkyl, or (hydroxy-C₃₋₆-cycloalkyl)-C₁₋₃-alkyl, and
the heterocycle formed by the group R¹R²N— may be mono-, di-, or trisubstituted by identical or different radicals R¹⁴. The substituents R¹⁴ preferably denote independently of one another F, Cl, Br, OH, C₁₋₄-alkyl, C₁₋₄-alkoxy, C₁₋₄-alkoxy-C₁₋₃-alkyl, hydroxy-C₁₋₄-alkyl, or CF₃, particularly hydroxy, C₁₋₃-alkyl, CF₃, or hydroxy-C₁₋₃-alkyl.

If the partial formulae shown above are substituted as specified, the following definitions of the group R¹R²N— are particularly preferred: hydroxypyrrolidinyl, hydroxypiperidinyl, 3,4-dihydroxypyrrolidinyl, 3,4-dihydroxypiperidinyl, 3,5-dihydroxypiperidinyl, (hydroxymethyl)pyrrolidinyl, (hydroxymethyl)piperidinyl, (hydroxymethyl)hydroxypyrrolidinyl, or (hydroxymethyl)hydroxypiperidinyl,
while in the groups specified a hydroxymethyl group at the C atom may be mono- or disubstituted by methyl, while two methyl substituents may be joined together, forming a cyclopropyl group, and
while in one or two hydroxy groups the H atom may be replaced by a methyl group, and
the groups specified do not have any other substituents or have one or two substituents selected independently of one another from fluorine, hydroxy, C₁₋₃-alkyl, hydroxy-C₁₋₃-alkyl, and CF₃.

The following partial formulae are most particularly preferred definitions of the abovementioned heterocyclic group 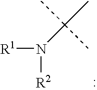 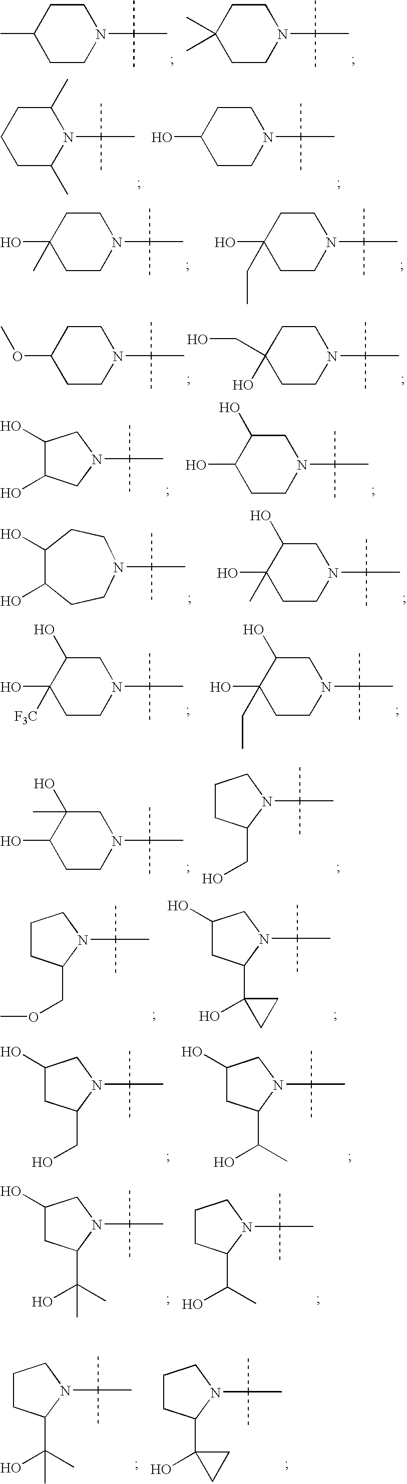 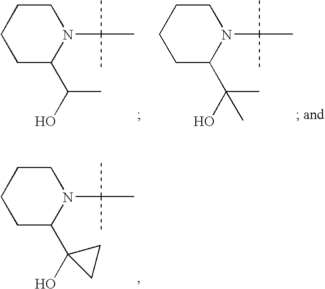
wherein the groups specified are not further substituted, or
wherein methyl or ethyl groups may be mono-, di or trisubstituted by fluorine, and wherein one or more H atoms of the heterocycle formed by the group R¹R²N— which are bound to carbon may be substituted independently of one another by fluorine, chlorine, CN, CF₃, C₁₋₃-alkyl, or hydroxy-C₁₋₃-alkyl, particularly C₁₋₃-alkyl or CF₃, preferably methyl, ethyl, or CF₃.

Among the preferred and particularly preferred meanings of R¹R²N— listed above, the following definitions of the substituent R¹⁴ are preferred: F, Cl, Br, cyano, C₁₋₄-alkyl, C₂₋₄-alkenyl, C₂₋₄-alkynyl, C₃₋₇-cycloalkyl, C₃₋₇-cycloalkyl-C₁₋₃-alkyl, hydroxy, hydroxy-C₁₋₃-alkyl, C₁₋₄-alkoxy, ω-(C₁₋₄-alkoxy)-C₁₋₃-alkyl, C₁₋₄-alkyl-carbonyl, carboxy, C₁₋₄-alkoxycarbonyl, hydroxy-carbonyl-C₁₋₃-alkyl, C₁₋₄-alkoxycarbonyl-C₁₋₃-alkyl, C₁₋₄-alkoxy-carbonylamino, C₁₋₄-alkoxy-carbonylamino-C₁₋₃-alkyl, amino, C₁₋₄-alkyl-amino, C₃₋₇-cycloalkyl-amino, N—(C₃₋₇-cycloalkyl)-N—(C₁₋₄-alkyl)-amino, di-(C₁₋₄-alkyl)-amino, cyclo-C₃₋₆-alkyleneimino, amino-C₁₋₃-alkyl, C₁₋₄-alkyl-amino-C₁₋₃-alkyl, C₃₋₇-cycloalkyl-amino-C₁₋₃-alkyl, N—(C₃₋₇-cycloalkyl)-N—(C₁₋₄-alkyl)-amino-C₁₋₃-alkyl, di-(C₁₋₄-alkyl)-amino-C₁₋₃-alkyl, cyclo-C₃₋₆-alkyleneimino-C₁₋₃-alkyl, aminocarbonyl, C₁₋₄-alkyl-aminocarbonyl, C₃₋₇-cycloalkyl-aminocarbonyl, N—(C₃₋₇-cycloalkyl)-N—(C₁₋₄-alkyl)-aminocarbonyl, di-(C₁₋₄-alkyl)-aminocarbonyl, pyridinyloxy, pyridinylamino, and pyridinyl-C₁₋₃-alkyl-amino.

Particularly preferred meanings of the substituent R¹⁴ are F, Cl, Br, C₁₋₄-alkyl, hydroxy, hydroxy-C₁₋₃-alkyl, C₁₋₄-alkoxy, ω-(C₁₋₄-alkoxy)-C₁₋₃-alkyl, amino-C₁₋₃-alkyl, C₁₋₄-alkyl-amino-C₁₋₃-alkyl, C₃₋₇-cycloalkyl-amino-C₁₋₃-alkyl, N—(C₃₋₇-cycloalkyl)-N—(C₁₋₄-alkyl)-amino-C₁₋₃-alkyl, di-(C₁₋₄-alkyl)-amino-C₁₋₃-alkyl, cyclo-C₃₋₆-alkyleneimino-C₁₋₃-alkyl, aminocarbonyl, and pyridylamino.

In the abovementioned preferred meanings of R¹⁴ in each case one or more C atoms may additionally be mono- or polysubstituted by F and/or in each case one or two C atoms may independently of one another additionally be monosubstituted by Cl or Br. Thus, preferred meanings of R¹⁴ also include, for example, —CF₃, —OCF₃, CF₃—CO—, and CF₃—CHOH—.

Most particularly preferred meanings of the substituent R¹⁴ are C₁₋₃-alkyl, hydroxy-C₁₋₃-alkyl, methoxymethyl, hydroxy, CF₃, CF₃—CHOH, particularly hydroxy, methyl, ethyl, CF₃, and hydroxymethyl.

According to a preferred first embodiment the group X is a methylene, ethylene, or propylene bridge, particularly preferably a methylene or ethylene bridge, which is unsubstituted or substituted by one or two identical or different C₁₋₃-alkyl substituents and/or a substituent selected from C₂₋₆-alkenyl, C₂₋₆-alkynyl, C₃₋₆-cycloalkyl, or C₃₋₆-cycloalkyl-C₁₋₃-alkyl, while two alkyl substituents may be joined together to form a 3- to 6-membered cycloalkyl group or an alkyl and an alkenyl substituent may be joined together to form a 5- or 6-membered cycloalkenyl group. According to this embodiment, X representing methylene or ethylene is preferably unsubstituted or mono- or disubstituted by methyl, ethyl, or isopropyl, while two alkyl substituents may be linked together forming a C₃₋₆-cycloalkyl group.

Particularly preferably according to this embodiment X is a —CH₂— bridge, which is unsubstituted or mono- or disubstituted by methyl, while two methyl groups may be joined together forming a cyclopropyl group. Most particularly preferably X is an unsubstituted —CH₂— bridge.

Examples of particularly preferred meanings of X are therefore

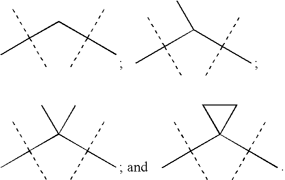

According to another embodiment, the group X is preferably an unbranched propylene or butylene bridge, while one or two C atoms may be monosubstituted by hydroxy, hydroxy-C₁₋₃-alkyl, or C₁₋₃-alkoxy, particularly hydroxy, and one or two C atoms may each be substituted by one or two identical or different C₁₋₃-alkyl substituents and/or a substituent selected from C₂₋₆-alkenyl, C₂₋₆-alkynyl, C₃₋₆-cycloalkyl, or C₃₋₆-cycloalkyl-C₁₋₃-alkyl, while two alkyl substituents may be joined together forming a 3- to 6-membered cycloalkyl group or an alkyl and an alkenyl substituent may be joined together forming a 5- or 6-membered cycloalkenyl group. In the group X, one or more C atoms may be mono- or polysubstituted by F and/or Cl, preferably F.

According to another embodiment, the group X is preferably an unbranched propylene or butylene bridge, wherein a —CH₂—CH₂— group not directly adjacent to the N atom of the R¹R²N— group is replaced by —CH═CH— or —C≡C—, particularly a —CH₂—CH═CH— or —CH₂—C≡C— bridge, while the meanings specified for X are unsubstituted or comprise one or two identical or different substituents selected independently of one another from fluorine and C₁₋₃-alkyl and/or a cyclopropyl substituent, while two alkyl groups may be joined together to form a C₃₋₆-cycloalkyl group.

Another preferred definition of X is also C₃₋₄-alkylene, particularly propylene, which has one or two identical or different substituents selected independently of one another from fluorine, chlorine, hydroxy, and C₁₋₃-alkyl and/or a cyclopropyl substituent, while two alkyl substituents may be joined together, forming a C₃₋₆-cycloalkyl group.

Preferred meanings under the definition of substituted propylene are, for example,

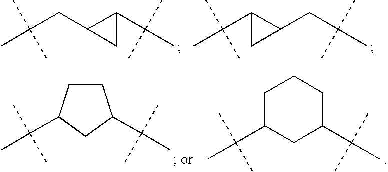

Other preferred meanings of X are —CH₂—CH═CH— or —CH₂—C≡C—, while the meanings specified for X are unsubstituted or have one or two identical or different substituents selected independently of one another from fluorine and C₁₋₃-alkyl and/or a cyclopropyl substituent, while two alkyl groups may be joined together forming a C₃₋₆-cycloalkyl group. Particularly preferably, the meaning given above for X are mono- or disubstituted by identical or different radicals selected from methyl, ethyl, and isopropyl, while two alkyl groups may be joined together as specified, to form a cyclic group.

Other preferred meanings of X are —CH₂—CH≡CH— and substituted —CH₂—CH═CH— selected from the group consisting of

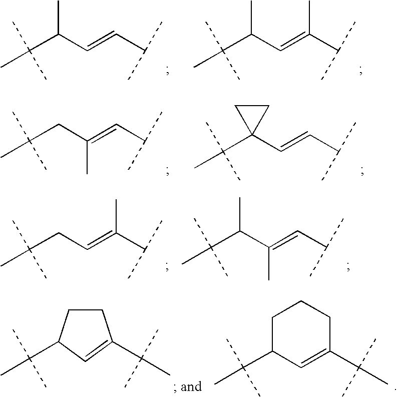

For X representing substituted alkenylene, only one of the two possible E/Z configurations is given above. Obviously, the other of the two E/Z configurations is also included according to the invention.

Preferred meanings of the group R¹⁰ are —OH, methoxy, and hydroxymethyl, particularly —OH.

The bridge W preferably denotes a single bond or ethylene, particularly preferably a single bond.

The bridge Z preferably denotes a single bond or ethylene, which may have one or two methyl substituents which may be joined together, forming a cyclopropyl group. Particularly preferably, Z denotes a single bond.

Preferred meanings of the group R^{M} in the group Y, in the event that M denotes NR^{M}, are selected from H, C₁₋₃-alkyl, prop-2-enyl, prop-2-ynyl, C₃₋₆-cycloalkyl, and C₃₋₆-cycloalkylmethyl. Most particularly preferably R^{M} denotes H or C₁₋₃-alkyl, particularly H or methyl.

The preferred alkyne compounds are those wherein M preferably denotes O or S.

Preferred definitions of the group Y are selected from the partial formulae 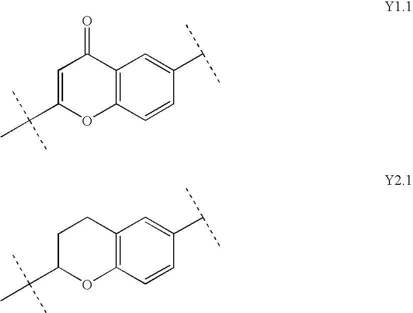
while both partial formulae are unsubstituted or in the two partial formulae one or more, preferably one or two C atoms are substituted independently of one another by R²⁰.

Particularly preferred substituents R²⁰ of the group Y are selected from the group consisting of fluorine, chlorine, bromine, cyano, nitro, C₁₋₄-alkyl, C₂₋₆-alkenyl, hydroxy, ω-hydroxy-C₁₋₃-alkyl, C₁₋₄-alkoxy, trifluoromethyl, trifluoromethoxy, C₂₋₄-alkynyl, C₁₋₄-alkoxycarbonyl, ω-(C₁₋₄-alkoxy)-C₁₋₃-alkyl, C₁₋₄-alkoxy-carbonylamino, amino, C₁₋₄-alkyl-amino, di-(C₁₋₄-alkyl)-amino, aminocarbonyl, C₁₋₄-alkyl-aminocarbonyl-, and di-(C₁₋₄-alkyl)-aminocarbonyl.

Most particularly preferred substituents R²⁰ of the group Y are selected from the group consisting of fluorine, chlorine, bromine, cyano, C₁₋₃-alkyl, C₂₋₃-alkenyl, C₂₋₃-alkynyl, C₁₋₃-alkoxy, C₁₋₄-alkoxycarbonyl, trifluoromethyl, and trifluoromethoxy, and in the case of a phenyl ring, nitro as well. Examples of particularly preferred meanings of the substituent R²⁰ are F, Cl, Br, methyl, ethyl, acetyl, or methoxy.

Preferably the group A is selected from among the bivalent cyclic groups phenyl, pyridinyl, pyrimidinyl, pyrazinyl, or pyridazinyl, which may be mono- or polysubstituted at one or more C atoms by identical or different radicals R²⁰, and in the case of a phenyl ring may also additionally be monosubstituted by nitro.

Most particularly preferably, A is one of the groups listed below 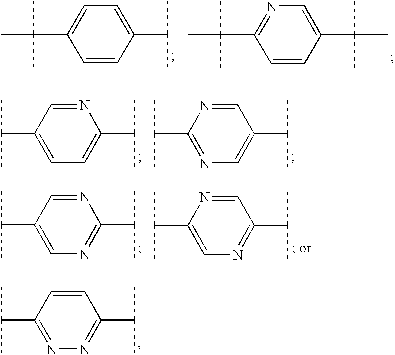
particularly 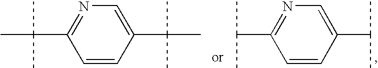
most particularly preferably 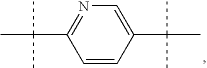
while the groups listed may be substituted as specified hereinbefore.

Particularly preferred substituents R²⁰ of the group A are, independently of one another, fluorine, chlorine, bromine, amino, CF₃, methoxy, and C₁₋₃-alkyl.

Preferably the group A is unsubstituted or monosubstituted by R²⁰, as specified.

Preferred definitions of the group B according to a first preferred embodiment are selected from the group consisting of phenyl, pyridyl, thienyl, and furanyl. Particularly preferably the group B denotes phenyl. The group B defined as specified may be mono- or polysubstituted by identical or different radicals R²⁰, while a phenyl group may additionally also be monosubstituted by nitro. Preferably the group B is unsubstituted or mono-, di-, or trisubstituted, particularly unsubstituted or mono- or disubstituted. In the case of a monosubstitution the substituent is preferably in the para position to the group A.

Preferred substituents R²⁰ of the group B are selected from the group consisting of fluorine, chlorine, bromine, cyano, nitro, C₁₋₄-alkyl, hydroxy, CHF₂, CHF₂—O— hydroxy-C₁₋₃-alkyl, C₁₋₄-alkoxy, trifluoromethyl, trifluoromethoxy, C₂₋₄-alkynyl, carboxy, C₁₋₄-alkoxycarbonyl, ω-(C₁₋₄-alkoxy)-C₁₋₃-alkyl, C₁₋₄-alkoxy-carbonylamino, amino, C₁₋₄-alkyl-amino, di-(C₁₋₄-alkyl)-amino, cyclo-C₃₋₆-alkyleneimino, aminocarbonyl, C₁₋₄-alkyl-aminocarbonyl, and di-(C₁₋₄-alkyl)-aminocarbonyl.

Particularly preferred substituents R²⁰ of the group B are selected from the group consisting of fluorine, chlorine, bromine, cyano, CF₃, C₁₋₃-alkyl, C₁₋₄-alkoxy, and trifluoromethoxy.

Most particularly preferred substituents R²⁰ of the group B are selected from the group consisting of chlorine and methoxy.

According to a second embodiment the definition of the group B is preferably selected from C₁₋₆-alkyl, C₂₋₆-alkenyl, C₂₋₆-alkynyl, C₃₋₇-cycloalkyl, C₅₋₇-cycloalkenyl, C₃₋₇-cycloalkyl-C₁₋₃-alkyl, C₃₋₇-cycloalkenyl-C₁₋₃-alkyl, C₃₋₇-cycloalkyl-C₁₋₃-alkenyl, or C₃₋₇-cycloalkyl-C₁₋₃-alkynyl, while one or more C atoms in the groups mentioned above for B may be mono- or polysubstituted by fluorine. In the cyclic groups according to the embodiment mentioned above, one or more C atoms may be substituted by R²⁰.

Particularly preferred according to this embodiment are the groups C₃₋₆-alkyl, C₃₋₆-alkenyl, C₃₋₆-alkynyl, cyclopentyl, cyclopentenyl, cyclohexyl, cyclohexenyl, cycloheptyl, cycloheptenyl, cyclopentyl-C₁₋₃-alkyl, cyclopentenyl-C₁₋₃-alkyl, cyclohexyl-C₁₋₃-alkyl, cyclohexenyl-C₁₋₃-alkyl, cycloheptyl-C₁₋₃-alkyl, and cycloheptenyl-C₁₋₃-alkyl, while one or more C atoms in the groups mentioned above for B may be mono- or polysubstituted by fluorine, and in cyclic groups one or more C atoms may be substituted by identical or different R²⁰.

Most particularly preferably, according to this second embodiment, B denotes cyclohexenyl, which is unsubstituted or contains 1, 2, or 3 identical or different substituents R²⁰, particularly methyl.

Some preferred definitions of other substituents according to the invention are listed below:
R⁴ preferably denotes H, C₁₋₄-alkyl, C₃₋₆-cycloalkyl, and C₃₋₆-cycloalkyl-methyl, particularly H, methyl, ethyl, propyl, isopropyl, n-propyl, cyclopropyl, cyclopentyl, cyclohexyl, cyclopropylmethyl, cyclopentylmethyl, and cyclohexylmethyl. Most particularly preferably R⁴ denotes H or methyl.

Preferably the substituent R¹³ has one of the meanings given for R¹⁶. Particularly preferably R¹³ denotes H, C₁₋₄-alkyl, C₃₋₇-cycloalkyl, C₃₋₇-cycloalkyl-C₁₋₃-alkyl, ω-hydroxy-C₂₋₃-alkyl, and ω-(C₁₋₄-alkoxy)-C₂₋₃-alkyl. Most particularly preferably R¹³ denotes H or C₁₋₄-alkyl. The alkyl groups specified above may be monosubstituted by Cl or mono- or polysubstituted by F.

Preferred meanings of the substituent R¹⁵ are H, C₁₋₄-alkyl, C₃₋₇-cycloalkyl, and C₃₋₇-cycloalkyl-C₁₋₃-alkyl, while, as hereinbefore defined, in each case one or more C atoms may additionally be mono- or polysubstituted by F and/or in each case one or two C atoms may independently of one another additionally be monosubstituted by Cl or Br. Particularly preferably, R¹⁵ denotes H, CF₃, methyl, ethyl, propyl, or butyl.

The substituent R¹⁶ preferably denotes H, C₁₋₄-alkyl, C₃₋₇-cycloalkyl, C₃₋₇-cycloalkyl-C₁₋₃-alkyl, ω-hydroxy-C₂₋₃-alkyl, or ω-(C₁₋₄-alkoxy)-C₂₋₃-alkyl, while, as hereinbefore defined, in each case one or more C atoms may additionally be mono- or polysubstituted by F and/or in each case one or two C atoms may independently of one another additionally be monosubstituted by Cl or Br. Particularly preferably R¹⁶ denotes H, CF₃, C₁₋₃-alkyl, C₃₋₆-cycloalkyl, or C₃₋₆-cycloalkyl-C₁₋₃-alkyl.

Preferably the substituent R¹⁷ has one of the meanings given as preferred for R¹⁶ or denotes phenyl, phenyl-C₁₋₃-alkyl, pyridinyl, or C₁₋₄-alkylcarbonyl. Particularly preferably R¹⁷ has one of the meanings given as preferred for R¹⁶.

Preferably one or both of the substituents R¹⁸ and R¹⁹ independently of one another denote hydrogen or C₁₋₄-alkyl, particularly hydrogen.

The substituent R²⁰ preferably denotes halogen, hydroxy, cyano, C₁₋₄-alkyl, C₂₋₄-alkenyl, C₂₋₄-alkynyl, C₃₋₇-cycloalkyl, C₃₋₇-cycloalkyl-C₁₋₃-alkyl, hydroxy-C₁₋₄-alkyl, R²²—C₁₋₃-alkyl, or one of the meanings given as preferred for R²², while, as hereinbefore defined, in each case one or more C atoms may additionally be mono- or polysubstituted by F and/or in each case one or two C atoms independently of one another may additionally be monosubstituted by Cl or Br.

Particularly preferred definitions of the group R²⁰ are halogen, hydroxy, cyano, C₁₋₄-alkyl, C₁₋₄-alkylcarbonyl, C₃₋₇-cycloalkyl, and C₁₋₄-alkoxy, while, as hereinbefore defined, in each case one or more C atoms may additionally be mono- or polysubstituted by F and/or in each case one or two C atoms independently of one another may additionally be monosubstituted by Cl or Br. Most particularly preferably R²⁰ denotes F, Cl, Br, I, OH, cyano, methyl, difluoromethyl, trifluoromethyl, ethyl, n-propyl, isopropyl, acetyl, methoxy, difluoromethoxy, trifluoromethoxy, ethoxy, n-propoxy, or isopropoxy.

The substituent R²² preferably denotes C₁₋₄-alkoxy, C₁₋₄-alkylthio, carboxy, C₁₋₄-alkylcarbonyl, C₁₋₄-alkoxycarbonyl, aminocarbonyl, C₁₋₄-alkylaminocarbonyl, di-(C₁₋₄-alkyl)-aminocarbonyl, C₁₋₄-alkyl-sulfonyl, C₁₋₄-alkyl-sulfinyl, C₁₋₄-alkyl-sulfonylamino, amino, C₁₋₄-alkylamino, di-(C₁₋₄-alkyl)-amino, C₁₋₄-alkyl-carbonylamino, hydroxy-C₁₋₃-alkylaminocarbonyl, aminocarbonylamino, or C₁₋₄-alkylaminocarbonyl-amino, while, as hereinbefore defined, in each case one or more C atoms may additionally be mono- or polysubstituted by F and/or in each case one or two C atoms may independently of one another additionally be monosubstituted by Cl or Br. Most particularly preferred meanings of R²² are C₁₋₄-alkoxy, C₁₋₄-alkylcarbonyl, amino, C₁₋₄-alkylamino, or di-(C₁₋₄-alkyl)-amino, wherein one or more H atoms may be replaced by fluorine.

Preferred definitions of the group R²¹ are C₁₋₄-alkyl, C₁₋₄-alkylcarbonyl, C₁₋₄-alkylsulfonyl, —SO₂—NH₂, —SO₂—NH—C₁₋₃-alkyl, —SO₂—N(C₁₋₃-alkyl)₂, and cyclo-C3-6-alkyleneimino-sulfonyl, while, as hereinbefore defined, in each case one or more C atoms may additionally be mono- or polysubstituted by F and/or in each case one or two C atoms independently of one another may additionally be monosubstituted by Cl or Br. Most particularly preferably R²¹ denotes C₁₋₄-alkyl or CF₃.

Cy preferably denotes a C₃₋₇-cycloalkyl, particularly a C₃₋₆-cycloalkyl group, a C₅₋₇-cycloalkenyl group, pyrrolidinyl, piperidinyl, piperazinyl, morpholinyl, thiomorpholinyl, aryl, or heteroaryl, and the abovementioned cyclic groups may be mono- or polysubstituted at one or more C atoms by identical or different radicals R²⁰, or in the case of a phenyl group may also additionally be monosubstituted by nitro, and/or one or more NH groups may be substituted by R²¹. Most particularly preferred definitions of the group Cy are C₃₋₆-cycloalkyl, pyrrolidinyl, and piperidinyl, which may be substituted as specified.

The term aryl preferably denotes phenyl or naphthyl, particularly phenyl.

The term heteroaryl preferably includes pyridyl, indolyl, quinolinyl, and benzoxazolyl.

Preferred compounds according to the invention are those wherein one or more of the groups, radicals, substituents and/or indices have one of the meanings specified hereinbefore as being preferred.

Particularly preferred compounds according to the invention may be described by one of the general formulae IIa to IId, particularly IIa and IIb: 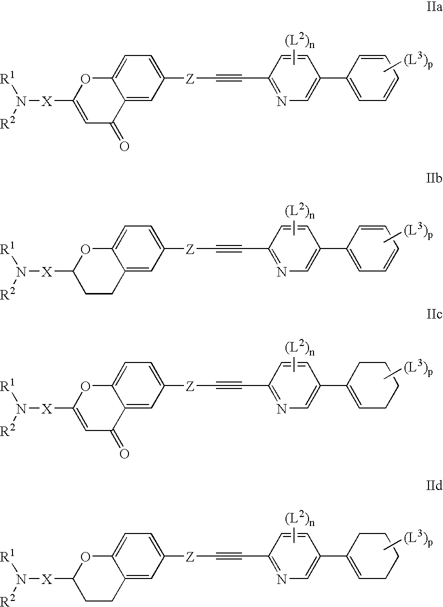
wherein:
the chromane and chromenone group is unsubstituted or mono- or disubstituted by L¹;
R¹

, R², X, and Z have one of the meanings given above; and
L¹, L², and L³ independently of one another have one of the meanings given for R²⁰, and
n and p independently of one another represent the values 0, 1, or 2, and p may also denote 3.

Preferably in formulae IIa to IId, particularly IIa and IIb:
X denotes an unsubstituted —CH₂— bridge or a —CH₂— bridge which is mono- or disubstituted by C₁₋₃-alkyl, particularly methyl, while two alkyl substituents may be joined together, forming a C₃₋₆-cycloalkyl group, particularly cyclopropyl;
Z denotes a single bond;
L¹ denotes fluorine, chlorine, bromine, cyano, C₁₋₃-alkyl, C₁₋₃-alkoxy, C₁₋₄-alkoxycarbonyl, trifluoromethyl, trifluoromethoxy, or nitro, particularly C₁₋₃-alkyl;
L² denotes fluorine, chlorine, bromine, CN, amino, CF₃, methoxy, or C₁₋₃-alkyl;
n denotes 0 or 1; and
L³ independently of one another have a meaning selected from among fluorine, chlorine, bromine, cyano, nitro, C₁₋₄-alkyl, hydroxy, co-hydroxy-C₁₋₃-alkyl, C₁₋₄-alkoxy, trifluoromethyl, trifluoromethoxy, C₂₋₄-alkynyl, carboxy, C₁₋₄-alkoxycarbonyl, ω-(C₁₋₄-alkoxy)-C₁₋₃-alkyl, C₁₋₄-alkoxy-carbonylamino, amino, C₁₋₄-alkyl-amino, di-(C₁₋₄-alkyl)-amino, cyclo-C₃₋₆-alkyleneimino, aminocarbonyl, C₁₋₄-alkyl-aminocarbonyl, or di-(C₁₋₄-alkyl)-aminocarbonyl, particularly preferably fluorine, chlorine, bromine, cyano, CF₃, C₁₋₃-alkyl, C₁₋₄-alkoxy, and trifluoromethoxy, with the proviso that a phenyl ring may only be monosubstituted by nitro, and
p denotes 0, 1, 2, or 3, particularly 1 or 2.

Most particularly preferably, in the formulae IIa, IIb, IIc, and IId:
R¹ and R² independently of one another denote C₁₋₄-alkyl, hydroxy-C₁₋₄-alkyl, C₃₋₅-alkenyl, C₃₋₅alkenyl, C₃₋₇-cycloalkyl, hydroxy-C₃₋₇-cycloalkyl, dihydroxy-C₃₋₆-alkyl, C₃₋₇-cycloalkyl-C₁₋₃-alkyl, tetrahydropyran-3-yl, tetrahydropyran-4-yl, (hydroxy-C₃₋₇-cycloalkyl)-C₁₋₃-alkyl, ω-(C₁₋₄-alkoxy)-C₂₋₃-alkyl, pyridyl, or benzyl, while an alkyl, cycloalkyl or cycloalkyl-alkyl group may additionally be mono- or disubstituted by hydroxy and/or hydroxy-C₁₋₃-alkyl, and/or mono- or polysubstituted by F or C₁₋₃-alkyl and/or monosubstituted by CF₃, Br, Cl, or CN, and one of the groups R¹ and R² may also denote H, and phenyl and pyridyl rings may be mono- or polysubstituted by identical or different radicals R²⁰, while phenyl may also be monosubstituted by nitro, or
R¹ and R² are joined together and form, together with the N atom to which they are bound, a heterocyclic group which is selected from pyrrolidine, piperidine, piperazine, wherein the free imine function is substituted by R¹³, and morpholine, wherein one or more H atoms may be replaced by identical or different groups R¹⁴, and while the heterocyclic group defined hereinbefore may be substituted through a single bond with a carbo- or heterocyclic group Cy, while Cy is selected from the group consisting of C₃₋₇-cycloalkyl and cyclo-C₃₋₆-alkyleneimino, while Cy may be mono- or polysubstituted by identical or different radicals R²⁰, where R²⁰ is as hereinbefore defined and is preferably selected from fluorine, CF₃, C₁₋₃-alkyl, hydroxy-C₁₋₃-alkyl, and hydroxy, and
R¹⁻⁴ is selected from F, Cl, Br, C₁₋₄-alkyl, hydroxy, hydroxy-C₁₋₃-alkyl, C₁₋₄-alkoxy, ω-(C₁₋₄-alkoxy)-C₁₋₃-alkyl, amino-C₁₋₃-alkyl, C₁₋₄-alkyl-amino-C₁₋₃-alkyl, C₃₋₇-cycloalkyl-amino-C₁₋₃-alkyl, N—(C₃₋₇-cycloalkyl)-N—(C₁₋₄-alkyl)-amino-C₁₋₃-alkyl, di-(C₁₋₄-alkyl)-amino-C₁₋₃-alkyl, cyclo-C₃₋₆-alkyleneimino-C₁₋₃-alkyl, aminocarbonyl, and pyridylamino, while in the abovementioned meanings one or more C atoms may each additionally be mono- or polysubstituted by F and/or in each case one or two C atoms may independently of one another additionally be monosubstituted by Cl or Br.

The compounds listed in the experimental section, including the tautomers, the diastereomers, the enantiomers, the mixtures thereof and the salts thereof, are preferred according to the invention.

Some expressions used hereinbefore and below to describe the compounds according to the invention will now be defined more fully.

The term halogen denotes an atom selected from among F, Cl, Br, and I, particularly F, Cl, and Br.

The term C₁₋ₙ-alkyl, where n has a value of 3 to 8, denotes a saturated, branched or unbranched hydrocarbon group with 1 to n C atoms. Examples of such groups include methyl, ethyl, n-propyl, isopropyl, butyl, isobutyl, sec-butyl, tert-butyl, n-pentyl, isopentyl, neopentyl, tert-pentyl, n-hexyl, isohexyl, etc.

The term C₁₋ₙ-alkylene, where n may have a value of 1 to 8, denotes a saturated, branched or unbranched hydrocarbon bridge with 1 to n C atoms. Examples of such groups include methylene (—CH₂—), ethylene (—CH₂—CH₂—), 1-methylethylene (—CH(CH₃)—CH₂—), 1,1-dimethylethylene (—C(CH₃)₂—CH₂—), n-prop-1,3-ylene (—CH₂—CH₂—CH₂—), 1-methylprop-1,3-ylene (—CH(CH₃)—CH₂—CH₂—), 2-methylprop-1,3-ylene (—CH₂—CH(CH₃)—CH₂—), etc., as well as the corresponding mirror-symmetrical forms.

The term C₂₋ₙ-alkenyl, where n has a value of 3 to 6, denotes a branched or unbranched hydrocarbon group with 2 to n C atoms and at least one C═C double bond. Examples of such groups include vinyl, 1-propenyl, 2-propenyl, isopropenyl, 1-butenyl, 2-butenyl, 3-butenyl, 2-methyl-1-propenyl, 1-pentenyl, 2-pentenyl, 3-pentenyl, 4-pentenyl, 3-methyl-2-butenyl, 1-hexenyl, 2-hexenyl, 3-hexenyl, 4-hexenyl, 5-hexenyl, etc.

The term C₂₋ₙ-alkynyl, where n has a value of 3 to 6, denotes a branched or unbranched hydrocarbon group with 2 to n C atoms and a C≡C triple bond. Examples of such groups include ethynyl, 1-propynyl, 2-propynyl, 1-butynyl, 2-butynyl, 3-butynyl, 2-methyl-1-propynyl, 1-pentynyl, 2-pentynyl, 3-pentynyl, 4-pentynyl, 3-methyl-2-butynyl, 1-hexynyl, 2-hexynyl, 3-hexynyl, 4-hexynyl, 5-hexynyl, etc.

The term C₁₋ₙ-alkoxy denotes a C₁₋ₙ-alkyl-O— group, wherein C₁₋ₙ-alkyl is defined as above. Examples of such groups include methoxy, ethoxy, n-propoxy, isopropoxy, n-butoxy, isobutoxy, sec-butoxy, tert-butoxy, n-pentoxy, isopentoxy, neopentoxy, tert-pentoxy, n-hexoxy, isohexoxy. etc.

The term C₁₋ₙ-alkylthio denotes a C₁₋ₙ-alkyl-S— group, wherein C₁₋ₙ-alkyl is defined as above. Examples of such groups include methylthio, ethylthio, n-propylthio, isopropylthio, n-butylthio, isobutylthio, sec-butylthio, tert-butylthio, n-pentylthio, isopentylthio, neopentylthio, tert-pentylthio, n-hexylthio, isohexylthio, etc.

The term C₁₋ₙ-alkylcarbonyl denotes a C₁₋ₙ-alkyl —C(═O)— group, wherein C₁₋ₙ-alkyl is defined as above. Examples of such groups include methylcarbonyl, ethylcarbonyl, n-propylcarbonyl, isopropylcarbonyl, n-butylcarbonyl, isobutylcarbonyl, sec-butylcarbonyl, tert-butylcarbonyl, n-pentylcarbonyl, isopentylcarbonyl, neopentylcarbonyl, tert-pentylcarbonyl, n-hexylcarbonyl, isohexylcarbonyl, etc.

The term C₃₋ₙ-cycloalkyl denotes a saturated mono-, bi-, tri-, or spirocarbocyclic, preferably monocarbocyclic group with 3 to n C atoms. Examples of such groups include cyclopropyl, cyclobutyl, cyclopentyl, cyclohexyl, cycloheptyl, cyclooctyl, cyclononyl, cyclododecyl, bicyclo[3,2,1]octyl, spiro[4,5]decyl, norpinyl, norbonyl, norcaryl, adamantyl, etc.

The term C₅₋ₙ-cycloalkenyl denotes a monounsaturated mono-, bi-, tri-. or spirocarbocyclic, preferably monocarboxylic group with 5 to n C atoms. Examples of such groups include cyclopentenyl, cyclohexenyl, cycloheptenyl, cyclooctenyl, cyclononenyl, etc.

The term C₃₋ₙ-cycloalkylcarbonyl denotes a C₃₋ₙ-cycloalkyl-C(═O) group, wherein C₃₋ₙ-cycloalkyl is as hereinbefore defined.

The term aryl denotes a carbocyclic, aromatic ring system, such as, for example, phenyl, biphenyl, naphthyl, anthracenyl, phenanthrenyl, fluorenyl, indenyl, pentalenyl, azulenyl, biphenylenyl, etc. A particularly preferred meaning of “aryl” is phenyl.

The term cyclo-C₃₋₆-alkyleneimino denotes a 4- to 7-membered ring which comprises 3 to 6 methylene units as well as an imino group, while the bond to the residue of the molecule is made via the imino group.

The term cyclo-C₃₋₆-alkyleneimino-carbonyl denotes a cyclo-C₃₋₆-alkyleneimino ring as hereinbefore defined which is linked to a carbonyl group via the imino group.

The term heteroaryl used in this application denotes a heterocyclic, aromatic ring system which comprises in addition to at least one C atom one or more heteroatoms selected from N, O, and/or S. Examples of such groups are furanyl, thiophenyl, pyrrolyl, oxazolyl, thiazolyl, imidazolyl, isoxazolyl, isothiazolyl, 1,2,3-triazolyl, 1,3,5-triazolyl, pyranyl, pyridyl, pyridazinyl, pyrimidinyl, pyrazinyl, 1,2,3-triazinyl, 1,2,4-triazinyl, 1,3,5-triazinyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4-thiadiazolyl, tetrazolyl, thiadiazinyl, indolyl, isoindolyl, benzofuranyl, benzothiophenyl (thianaphthenyl), indazolyl, benzimidazolyl, benzthiazolyl, benzisothiazolyl, benzoxazolyl, benzisoxazolyl, purinyl, quinazolinyl, quinozilinyl, quinolinyl, isoquinolinyl, quinoxalinyl, naphthyridinyl, pteridinyl, carbazolyl, azepinyl, diazepinyl, acridinyl, etc. The term heteroaryl also comprises the partially hydrogenated heterocyclic, aromatic ring systems, particularly those listed above. Examples of such partially hydrogenated ring systems are 2,3-dihydrobenzofuranyl, pyrolinyl, pyrazolinyl, indolinyl, oxazolidinyl, oxazolinyl, oxazepinyl, etc. Particularly preferably heteroaryl denotes a heteroaromatic mono- or bicyclic ring system.

Terms such as C₃₋₇-cycloalkyl-C₁₋ₙ-alkyl, heteroaryl-C₁₋ₙ-alkyl, etc. refer to C₁₋ₙ-alkyl, as defined above, which is substituted with a C₃₋₇-cycloalkyl, aryl, or heteroaryl group.

Many of the terms given above may be used repeatedly in the definition of a formula or group and in each case have one of the meanings given above, independently of one another. Thus, for example, in the group di-C₁₋₄-alkyl-amino, the two alkyl groups may have the same or different meanings.

The term “unsaturated”, for example, in “unsaturated carbocyclic group” or “unsaturated heterocyclic group”, as used particularly in the definition of the group Cy, comprises in addition to the mono- or polyunsaturated groups, the corresponding, totally unsaturated groups, but particularly the mono- and diunsaturated groups.

The term “optionally substituted” used in this application indicates that the group thus designated is either unsubstituted or mono- or polysubstituted by the substituents specified. If the group in question is polysubstituted, the substituents may be identical or different.

The style used hereinbefore and hereinafter, according to which in a cyclic group a bond of a substituent is shown towards the centre of this cyclic group, indicates unless otherwise stated that this substituent may be bound to any free position of the cyclic group carrying an H atom.

Thus in the example 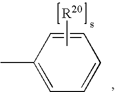
the substituent R²⁰ where s=1, may be bound to any of the free positions of the phenyl ring; where s=2, selected substituents R²⁰ may independently of one another be bound to different free positions of the phenyl ring.

An H atom bound to an N atom (imino or amino group) may in each case be replaced by a group which can be cleaved in vivo. By a group which can be cleaved in vivo from an N atom is meant, for example, a hydroxy group, an acyl group such as the benzoyl or pyridinoyl group, or a C₁₋₁₆-alkanoyl group such as the formyl, acetyl, propionyl, butanoyl, pentanoyl, or hexanoyl group, an allyloxycarbonyl group, a C₁₋₁₆-alkoxycarbonyl group such as the methoxycarbonyl, ethoxycarbonyl, propoxycarbonyl, isopropoxycarbonyl, butoxycarbonyl, tert-butoxycarbonyl, pentoxycarbonyl, hexyloxycarbonyl, octyloxycarbonyl, nonyloxycarbonyl, decyloxycarbonyl, undecyloxycarbonyl, dodecyloxycarbonyl, or hexadecyloxycarbonyl group, a phenyl-C₁₋₆-alkoxycarbonyl group such as the benzyloxycarbonyl, phenylethoxycarbonyl, or phenylpropoxycarbonyl group, a C₁₋₃-alkylsulfonyl-C₂₋₄-alkoxycarbonyl, C₁₋₃-alkoxy-C₂₋₄-alkoxy-C₂₋₄-alkoxycarbonyl, or RₑCO—O—(R_{f}CR_{b})—O—CO— group wherein
Rₑ denotes a C₁₋₈-alkyl, C₅₋₇-cycloalkyl, phenyl, or phenyl-C₁₋₃-alkyl group,
R_{f} denotes a hydrogen atom, a C₁₋₃-alkyl, C₅₋₇-cycloalkyl, or phenyl group, and
R_{g} denotes a hydrogen atom, a C₁₋₃-alkyl or RₑCO—O—(R_{f}CR_{g})—O— group wherein Rₑ to R_{g} are as hereinbefore defined,
   while the phthalimido group is an additional possibility for an amino group, and the abovementioned ester groups may also be used as a group which can be converted in vivo into a carboxy group.

The residues and substituents described above may be mono- or polysubstituted by fluorine as described. Preferred fluorinated alkyl groups are fluoromethyl, difluoromethyl, and trifluoromethyl. Preferred fluorinated alkoxy groups are fluoromethoxy, difluoromethoxy and trifluoromethoxy. Preferred fluorinated alkylsulfinyl and alkylsulfonyl groups are trifluoromethylsulfinyl and trifluoromethylsulfonyl.

The compounds of general formula I according to the invention may have acid groups, predominantly carboxyl groups, and/or basic groups such as, e.g., amino functions. Compounds of general formula I may therefore be present as internal salts, as salts with pharmaceutically useable inorganic acids such as hydrochloric acid, sulfuric acid, phosphoric acid, sulfonic acid or organic acids (such as, for example, maleic acid, fumaric acid, citric acid, tartaric acid, or acetic acid) or as salts with pharmaceutically useable bases such as alkali or alkaline earth metal hydroxides or carbonates, zinc or ammonium hydroxides or organic amines such as, e.g., diethylamine, triethylamine, and triethanolamine inter alia.

The compounds according to the invention may be obtained using methods of synthesis which are known in principle. Preferably the compounds are obtained analogously to the methods of preparation explained more fully hereinafter.

The two reaction plans A and B that follow show the synthesis of the compounds A.5 and B.5 according to the invention, while R¹, R², X, Y, Z, W, A, and B have one of the meanings described hereinbefore. Hal denotes chlorine, bromine, or iodine, particularly bromine or iodine, particularly preferably iodine.

According to Reaction Plan A, the halogen compound A.1 is reacted with the alkyne compound A.2 in a molar ratio of about 1.5:1 to 1:1.5 under a protective has atmosphere in the presence of a suitable palladium catalyst, a suitable base and copper (I) iodide in a suitable solvent.

A preferred amount of copper (I) iodide is in the range from 1 to 15 mol %, particularly 5 to 10 mol % based on the educt A.1.

Suitable palladium catalysts are, for example, Pd(PPh₃)₄, Pd₂(dba)₃, Pd(OAc)₂, Pd(PPh₃)₂Cl₂, Pd(CH₃CN)₂Cl₂, or Pd(dppf)Cl₂. The palladium catalyst is preferably used in an amount from 1 to 15 mol %, particularly 5 to 10 mol % based on the educt A.1.

Suitable bases are particularly amines, such as, for example, triethylamine or ethyldiisopropylamine, as well as Cs₂CO₃. The base is preferably used in an at least equimolar amount based on the educt A.1, in excess or as the solvent. Moreover, suitable solvents are dimethylformamide or ether, such as, for example, tetrahydrofuran, including the mixtures thereof. The reaction takes place over a period of about 2 to 24 hours in a temperature range of about 20° C. to 90° C.

The alkyne compound A.3 obtained is reacted directly or after prior purification with methanesulfonic acid chloride to form the methanesulfonate derivative A.4. The reaction conditions required are known to the skilled person as such. Advantageous solvents are halogenated hydrocarbons, such as, for example, dichloromethane. Suitable reaction temperatures are usually in the range from 0° C. to 30° C.

The reaction solution containing the methanesulfonate derivative A.4 or the purified methanesulfonate derivative A.4, dissolved in a suitable solvent, is reacted with an amine H—NR¹R² to yield the end product A.5 and then optionally purified. If the amine H—NR¹R² has another primary or secondary amino function, this is advantageously provided with a protective group beforehand, which can be cleaved again after the reaction has ended, using methods known from the literature. The product thus obtained may, for example, be converted into the salt form by reaction with a corresponding acid. A preferred molar ratio of the derivative A.4 to the amine compound is in the range from 1.5:1 to 1:1.5. Suitable solvents are dimethylformamide or ether, such as, for example, tetrahydrofuran, including the mixtures thereof.

The reaction to form the product A.5 is advantageously carried out in a temperature range from about 20° C. to 90° C.

Reaction Plan A:

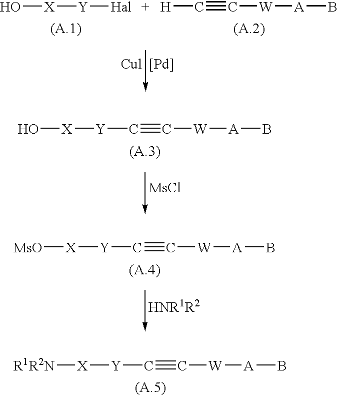

According to Reaction Plan B, the halogen compound B.2 is reacted with the alkyne compound B.1 in a molar ratio of about 1.5:1 to 1:1.5 under a protective gas atmosphere in the presence of a suitable palladium catalyst, a suitable base, and copper (I) iodide in a suitable solvent. Information on suitable reaction conditions, including catalysts, bases, and solvents, may be found in the explanations of Reaction Plan A.

The alkyne compound B.3 obtained is reacted directly, or after prior purification, with methanesulfonic acid chloride to form the methanesulfonate derivative B.4. The reaction conditions to be respected can again be found in the remarks accompanying Reaction Plan A.

The reaction solution containing the methanesulfonate derivative B.4 or the purified methanesulfonate derivative B.4, dissolved in a suitable solvent, is reacted with an amine H—NR¹R² to form the end product B.5 and then optionally purified. Here again, the remarks concerning Reaction Plan A apply.

Reaction Plan B:

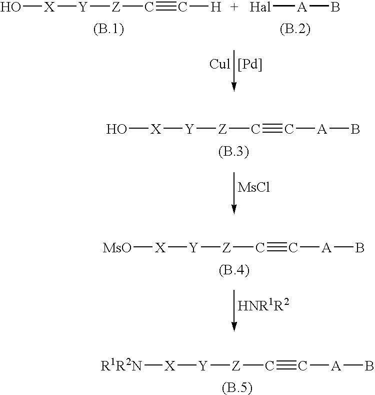

According to the other Reaction Plan C, the halogen compound C.1 is reacted with the alkyne compound C.2 in a molar ratio of about 1.5:1 to 1:1.5 under a protective gas atmosphere in the presence of a suitable palladium catalyst, a suitable base and copper (I) iodide in a suitable solvent to form the product C.3 directly. Information on suitable reaction conditions, including catalysts, bases, and solvents, may be found in the explanatory remarks accompanying Reaction Plan A.

Reaction Plan C:

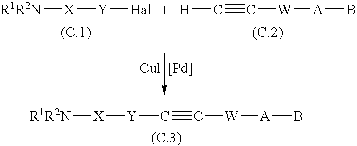

An alternative method of synthesis to this is shown in Reaction Plan D. According to this, the halogen compound D.2 is reacted with the alkyne compound D.1 in a molar ratio of about 1.5:1 to 1:1.5 under a protective gas atmosphere in the presence of a suitable palladium catalyst, a suitable base and copper (I) iodide in a suitable solvent to form the product D.3 directly. Once again, information on suitable reaction conditions, including catalysts, bases, and solvents, may be found in the explanatory remarks accompanying Reaction Plan A.

Reaction Plan D:

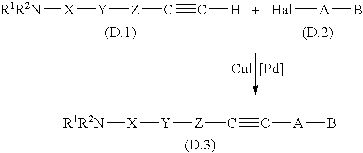

The reactions according to Plans A, B, C, and D are particularly advantageously carried out with the corresponding iodine compounds A.1, B.2, C.1, and D.2. In the event that Hal denotes bromine in compounds A.1, B.2, C.1, or D.2, it is advantageous to convert it into the corresponding iodine compound beforehand. One particularly advantageous method is the Aryl-Finkelstein reaction (Artis Klapars and Stephen L. Buchwald, Copper-Catalyzed Halogen Exchange in Aryl Halides: An Aromatic Finkelstein Reaction, J. Am. Chem. Soc. (2002), 124 (50), pp. 1-4844-1-4845). Thus, for example, the halogen compound A.1, B.2, C.1, or D.2 may be reacted with sodium iodide in the presence of N,N′-dimethylethylenediamine and copper (I) iodide in a suitable solvent to form the corresponding iodine compound. An advantageous molar ratio of the halogen compound to sodium iodide is 1:1.8 to 1:2.3. N,N′-dimethylethylenediamine is advantageously used in a molar ratio of 10 to 30 mol % based on the halogen compound A.1, B.2, C.1, or D.2. Preferred amounts of copper (I) iodide are in the range from 5 to 20 mol % based on the halogen compound A.1, B.2, C.1, or D.2. A suitable solvent is, for example, 1,4-dioxane. Suitable reaction temperatures are in the range from about 20° C. to 110° C. The reaction is substantially complete after 2 to 72 hours.

The compounds according to the invention may be obtained using methods of synthesis which are known in principle. Preferably the compounds are obtained analogously to the methods of preparation explained more fully in the experimental section.

Stereoisomeric compounds of formula (I) may chiefly be separated by conventional methods. The diastereomers are separated on the basis of their different physico-chemical properties, e.g., by fractional crystallization from suitable solvents, by high pressure liquid or column chromatography, using chiral or preferably non-chiral stationary phases.

Racemates covered by general formula (I) may be separated, for example, by HPLC on suitable chiral stationary phases (e.g., Chiral AGP, CHIRALPAK® AD). Racemates which contain a basic or acidic function can also be separated via the diastereomeric, optically active salts which are produced on reacting with an optically active acid, for example, (+) or (−)-tartaric acid, (+) or (−)-diacetyl tartaric acid, (+) or (−)-monomethyl tartrate, or (+)-camphorsulfonic acid, or an optically active base, for example, with (R)-(+)-1-phenylethylamine, (S)-(−)-1-phenylethylamine, or (S)-brucine.

According to a conventional method of separating isomers, the racemate of a compound of formula (I) is reacted with one of the abovementioned optically active acids or bases in equimolar amounts in a solvent and the resulting crystalline, diastereomeric, optically active salts thereof are separated using their different solubilities. This reaction may be carried out in any type of solvent provided that it is sufficiently different in terms of the solubility of the salts. Preferably, methanol, ethanol or mixtures thereof, for example, in a ratio by volume of 50:50, are used. Then each of the optically active salts is dissolved in water, carefully neutralized with a base such as sodium carbonate or potassium carbonate, or with a suitable acid, e.g., with dilute hydrochloric acid or aqueous methanesulfonic acid and in this way the corresponding free compound is obtained in the (+) or (−) form.

The (R) or (S) enantiomer alone or a mixture of two optically active diastereomeric compounds of general formula (I) may also be obtained by performing the syntheses described above with a suitable reaction component in the (R) or (S) configuration.

As already mentioned, the compounds of formula (I) may be converted into the salts thereof, particularly for pharmaceutical use into the physiologically and pharmacologically acceptable salts thereof. These salts may be present on the one hand as physiologically and pharmacologically acceptable acid addition salts of the compounds of formula (I) with inorganic or organic acids. On the other hand, in the case of acidically bound hydrogen, the compound of formula (I) may also be converted by reaction with inorganic bases into physiologically and pharmacologically acceptable salts with alkali or alkaline earth metal cations as counter-ion. The acid addition salts may be prepared, for example, using hydrochloric acid, hydrobromic acid, sulfuric acid, phosphoric acid, methanesulfonic acid, ethanesulfonic acid, toluenesulfonic acid, benzenesulfonic acid, acetic acid, fumaric acid, succinic acid, lactic acid, citric acid, tartaric acid, or maleic acid. Moreover, mixtures of the abovementioned acids may be used. To prepare the alkali and alkaline earth metal salts of the compound of formula (I) with acidically bound hydrogen the alkali and alkaline earth metal hydroxides and hydrides are preferably used, while the hydroxides and hydrides of the alkali metals, particularly of sodium and potassium, are preferred and sodium and potassium hydroxide are most preferred.

The compounds according to the present invention, including the physiologically acceptable salts, are effective as antagonists of the MCH receptor, particularly the MCH-1 receptor, and exhibit good affinity in MCH receptor binding studies. Pharmacological test systems for MCH-antagonistic properties are described in the following experimental section.

As antagonists of the MCH receptor the compounds according to the invention are advantageously suitable as pharmaceutical active substances for the prevention and/or treatment of symptoms and/or diseases caused by MCH or causally connected with MCH in some other way. Generally the compounds according to the invention have low toxicity, they are well absorbed by oral route and have good intracerebral transitivity, particularly brain accessibility.

Therefore, MCH antagonists which contain at least one compound according to the invention are particularly suitable in mammals, such as, for example, rats, mice, guinea pigs, hares, dogs, cats, sheep, horses, pigs, cattle, monkeys, and humans, for the treatment and/or prevention of symptoms and/or diseases which are caused by MCH or are otherwise causally connected with MCH.

Diseases caused by MCH or otherwise causally connected with MCH are particularly metabolic disorders, such as, for example, obesity, and eating disorders, such as, for example, bulimia, including bulimia nervosa. The indication obesity includes in particular exogenic obesity, hyperinsulinemic obesity, hyperplasmic obesity, hyperphyseal adiposity, hypoplasmic obesity, hypothyroid obesity, hypothalamic obesity, symptomatic obesity, infantile obesity, upper body obesity, alimentary obesity, hypogonadal obesity, central obesity. This range of indications also includes cachexia, anorexia, and hyperphagia.

Compounds according to the invention may be particularly suitable for reducing hunger, curbing appetite, controlling eating behavior and/or inducing a feeling of satiation.

In addition, the diseases caused by MCH or otherwise causally connected with MCH also include hyperlipidemia, cellulitis, fatty accumulation, malignant mastocytosis, systemic mastocytosis, emotional disorders, affectivity disorders, depression, anxiety states, reproductive disorders, sexual disorders, memory disorders, epilepsy, forms of dementia, and hormonal disorders.

Compounds according to the invention are also suitable as active substances for the prevention and/or treatment of other illnesses and/or disorders, particularly those which accompany obesity, such as, for example, diabetes, diabetes mellitus, particularly type II diabetes, hypoglycemia, particularly chronic hyperglycemia, complications of diabetes including diabetic retinopathy, diabetic neuropathy, diabetic nephropathy, etc., insulin resistance, pathological glucose tolerance, encephalorrhagia, cardiac insufficiency, cardiovascular diseases, particularly arteriosclerosis and high blood pressure, arthritis, and gonitis.

MCH antagonists and formulations according to the invention may advantageously be used in combination with a dietary therapy, such as, for example, a dietary diabetes treatment, and exercise.

Another range of indications for which the compounds according to the invention are advantageously suitable is the prevention and/or treatment of micturition disorders, such as, for example, urinary incontinence, hyperactive bladder, urgency, nycturia, and enuresis, while the hyperactive bladder and urgency may or may not be connected with benign prostatic hyperplasia.

Generally speaking, the compounds according to the invention are potentially suitable for preventing and/or treating dependencies, such as, for example, alcohol and/or nicotine dependency, and/or withdrawal symptoms, such as, for example, weight gain in smokers coming off nicotine. By “dependency” is generally meant here an irresistible urge to take an addictive substance and/or to perform certain actions, particularly in order to either achieve a feeling of wellbeing or to eliminate negative emotions. In particular, the term “dependency” is used here to denote a dependency on an addictive substance. By “withdrawal symptoms” are meant here, in general, symptoms which occur or may occur when addictive substances are withdrawn from patients dependent on one or more such substances. The compounds according to the invention are potentially suitable particularly as active substances for reducing or ending tobacco consumption, for the treatment or prevention of a nicotine dependency and/or for the treatment or prevention of nicotine withdrawal symptoms, for reducing the craving for tobacco and/or nicotine and generally as an anti-smoking agent. The compounds according to the invention may also be useful for preventing or at least reducing the weight gain typically seen when smokers are coming off nicotine. The substances may also be suitable as active substances which prevent or at least reduce the craving for and/or relapse into a dependency on addictive substances. The term addictive substances refers particularly but not exclusively to substances with a psycho-motor activity, such as narcotics or drugs, particularly alcohol, nicotine, cocaine, amphetamine, opiates, benzodiazepines, and barbiturates.

The dosage required to achieve such an effect is conveniently, by intravenous or subcutaneous route, 0.001 to 30 mg/kg of body weight, preferably 0.01 to 5 mg/kg of body weight, and by oral or nasal route or by inhalation, 0.01 to 50 mg/kg of body weight, preferably 0.1 to 30 mg/kg of body weight, in each case 1 to 3× daily.

For this purpose, the compounds prepared according to the invention may be formulated, optionally in conjunction with other active substances as described hereinafter, together with one or more inert conventional carriers and/or diluents, e.g., with corn starch, lactose, glucose, microcrystalline cellulose, magnesium stearate, polyvinylpyrrolidone, citric acid, tartaric acid, water, water/ethanol, water/glycerol, water/sorbitol, water/polyethylene glycol, propylene glycol, cetylstearyl alcohol, carboxymethylcellulose, or fatty substances such as hard fat or suitable mixtures thereof, to produce conventional galenic preparations such as plain or coated tablets, capsules, lozenges, powders, granules, solutions, emulsions, syrups, aerosols for inhalation, ointments, or suppositories.

In addition to pharmaceutical compositions, the invention also includes compositions containing at least one alkyne compound according to the invention and/or a salt according to the invention optionally together with one or more physiologically acceptable excipients. Such compositions may also be, for example, foodstuffs which may be solid or liquid, in which the compound according to the invention is incorporated.

For the abovementioned combinations it is possible to use as additional active substances particularly those which, for example, potentiate the therapeutic effect of an MCH antagonist according to the invention in terms of one of the indications mentioned above and/or which make it possible to reduce the dosage of an MCH antagonist according to the invention. Preferably one or more additional active substances are selected from among active substances for the treatment of diabetes, active substances for the treatment of diabetic complications, active substances for the treatment of obesity, preferably other than MCH antagonists, active substances for the treatment of high blood pressure, active substances for the treatment of hyperlipidemia, including arteriosclerosis, active substances for the treatment of dyslipidemia, including arteriosclerosis, active substances for the treatment of arthritis, active substances for the treatment of anxiety states, and active substances for the treatment of depression.

The abovementioned categories of active substances will now be explained in more detail by means of examples.

Examples of active substances for the treatment of diabetes are insulin sensitizers, insulin secretion accelerators, biguanides, insulins, α-glucosidase inhibitors, and β₃ adrenoreceptor agonists. Insulin sensitizers include glitazones, particularly pioglitazone and its salts (preferably hydrochloride), troglitazone, rosiglitazone and its salts (preferably maleate), JTT-501, GI-262570, MCC-555, YM-440, DRF-2593, BM-13-1258, KRP-297, R-119702, and GW-1929. Insulin secretion accelerators include sulfonylureas, such as, for example, tolbutamide, chloropropamide, tolazamide, acetohexamide, glyclopyramide and its ammonium salts, glibenclamide, gliclazide, and glimepiride. Further examples of insulin secretion accelerators are repaglinide, nateglinide, mitiglinide (KAD-1229), and JTT-608. Biguanides include metformin, buformin, and phenformin. Insulins include those obtained from animals, particularly cattle or pigs, semisynthetic human insulins which are synthesized enzymatically from insulin obtained from animals, human insulin obtained by genetic engineering, e.g., from Escherichia coli or yeasts. Moreover, the term insulin also includes insulin-zinc (containing 0.45 to 0.9 percent by weight of zinc) and protamine-insulin-zinc obtainable from zinc chloride, protamine sulfate, and insulin. Insulin may also be obtained from insulin fragments or derivatives (for example INS-1, etc.). Insulin may also include different kinds, e.g., with regard to the onset time and duration of effect (“ultra immediate action type”, “immediate action type”, “two phase type”, “intermediate type”, “prolonged action type”, etc.), which are selected depending on the pathological condition of the patient. α-Glucosidase inhibitors include acarbose, voglibose, miglitol, and emiglitate. β₃ Adrenoreceptor agonists include AJ-9677, BMS-196085, SB-226552, and AZ401-40. Active substances for the treatment of diabetes other than those mentioned above include ergoset, pramlintide, leptin, and BAY-27-9955 as well as glycogen phosphorylase inhibitors, sorbitol dehydrogenase inhibitors, protein tyrosine phosphatase IB inhibitors, dipeptidyl protease inhibitors, glipazide, and glyburide.

Active substances for the treatment of diabetic complications include, for example, aldose reductase inhibitors, glycation inhibitors and protein kinase C inhibitors, DPP-IV blockers, GLP-1 or GLP-2 analogues, and SGLT-2 inhibitors. Aldose reductase inhibitors are, for example, tolrestat, epalrestat, imirestat, zenarestat, SNK-860, zopolrestat, ARI-50i, AS-3201. An example of a glycation inhibitor is pimagedine. Protein Kinase C inhibitors are, for example, NGF, and LY-333531. DPP-IV blockers are, for example, LAF237 (Novartis), MK431 (Merck), as well as 815541, 823093, and 825964 (all GlaxoSmithKline). GLP-1 analogues are, for example, Liraglutide (NN2211) (Novo Nordisk), CJC-1131 (Conjuchem), and Exenatide (Amylin). SGLT-2 inhibitors are, for example, AVE-2268 (Aventis) and T-1095 (Tanabe, Johnson & Johnson). Active substances other than those mentioned above for the treatment of diabetic complications include alprostadil, thiapride hydrochloride, cilostazol, mexiletine hydrochloride, ethyl eicosapentate, memantine, and pimagedine (ALT-711).

Active substances for the treatment of obesity, preferably other than MCH antagonists, include lipase inhibitors and anorectics. A preferred example of a lipase inhibitor is orlistat. Examples of preferred anorectics are phentermine, mazindol, fluoxetine, sibutramine, baiamine, (S)-sibutramine, SR-1-41716, and NGD-95-1. Active substances other than those mentioned above for the treatment of obesity include lipstatin.

Moreover, for the purposes of this application, the active substance group of anti-obesity active substances also includes the anorectics, of which the β₃ agonists, thyromimetic active substances and NPY antagonists should be emphasized. The range of substances which may be considered as preferred anti-obesity or anorectic active substances is indicated by the following additional list, by way of example: phenylpropanolamine, ephedrine, pseudoephedrine, phentermine, a cholecystokinin-A (hereinafter referred to as CCK-A) agonist, a monoamine reuptake inhibitor (such as, for example, sibutramine), a sympathomimetic active substance, a serotonergic active substance (such as, for example, dexfenfluramine, fenfluramine, a 5-HT2C agonist such as BVT.933 or APD356, or duloxetine), a dopamine antagonist (such as, for example, bromocriptine or pramipexole), a melanocyte-stimulating hormone receptor agonist or mimetic, an analogue of melanocyte-stimulating hormone, a cannabinoid receptor antagonist (ACOMPLIA® (rimonabant)), an MCH antagonist, the OB protein (hereinafter referred to as leptin), a leptin analogue, a fatty acid synthase (FAS) antagonist, a leptin receptor agonist, a galanine antagonist, and a GI lipase inhibitor or reducer (such as, for example, orlistat). Other anorectics include bombesin agonists, dehydroepiandrosterone or its analogues, glucocorticoid receptor agonists and antagonists, orexin receptor antagonists, urocortin binding protein antagonists, agonists of the Glucagon-like Peptide-1 receptor, such as, for example, exendin, AC 2993, CJC-1131, IP10, or GRT0203Y, DPP-IV inhibitors and ciliary neurotrophic factors, such as, for example, axokines. In this context mention should also be made of the forms of therapy which produce weight loss by increasing the fatty acid oxidation in the peripheral tissue, such as, for example, inhibitors of acetyl-CoA-carboxylase.

Active substances for the treatment of high blood pressure include inhibitors of angiotensin converting enzyme, calcium antagonists, potassium channel openers, and angiotensin II antagonists. Inhibitors of angiotensin converting enzyme include captopril, enalapril, alacepril, delapril (hydrochloride), lisinopril, imidapril, benazepril, cilazapril, temocapril, trandolapril, and manidipine (hydrochloride). Examples of calcium antagonists are nifedipine, amlodipine, efonidipine, and nicardipine. Potassium channel openers include levcromakalim, L-27152, AL0671, and NIP-121. Angiotensin II antagonists include telmisartan, losartan, candesartan cilexetil, valsartan, irbesartan, CS-866, and E4177.

Active substances for the treatment of hyperlipidemia, including arteriosclerosis, include HMG-CoA reductase inhibitors and fibrate compounds. HMG-CoA reductase inhibitors include pravastatin, simvastatin, lovastatin, atorvastatin, fluvastatin, lipantil, cerivastatin, itavastatin, ZD-4522 and their salts. Fibrate compounds include bezafibrate, clinofibrate, clofibrate, and simfibrate.

Active substances for the treatment of dyslipidemia, including arteriosclerosis, include, e.g., medicaments which raise the HDL level, such as, e.g., nicotinic acid and derivatives and preparations thereof, such as, e.g., niaspan, as well as agonists of the nicotinic acid receptor.

Active substances for the treatment of arthritis include NSAIDs (non-steroidal anti-inflammatory drugs), particularly COX-2 inhibitors, such as, for example, meloxicam or ibuprofen.

Active substances for the treatment of anxiety states include chlordiazepoxide, diazepam, oxozolam, medazepam, cloxazolam, bromazepam, lorazepam, alprazolam, and fludiazepam.

Active substances for the treatment of depression include fluoxetine, fluvoxamine, imipramine, paroxetine, and sertraline.

The dosage for these active substances is conveniently 1/5 of the lowest normal recommended dose up to 1/1 of the normal recommended dose.

In another embodiment, the invention also relates to the use of at least one alkyne compound according to the invention and/or a salt according to the invention for influencing the eating behavior of a mammal. This use is particularly based on the fact that compounds according to the invention may be suitable for reducing hunger, curbing appetite, controlling eating behavior and/or inducing a feeling of satiety. The eating behavior is advantageously influenced so as to reduce food intake. Therefore, the compounds according to the invention are advantageously used for reducing body weight. Another use according to the invention is the prevention of increases in body weight, for example, in people who had previously taken steps to lose weight and are interested in maintaining their lower body weight. According to this embodiment it is preferably a non-therapeutic use. Such a non-therapeutic use might be a cosmetic use, for example, to alter the external appearance, or an application to improve general health. The compounds according to the invention are preferably used non-therapeutically for mammals, particularly humans, not suffering from any diagnosed eating disorders, no diagnosed obesity, bulimia, diabetes and/or no diagnosed micturition disorders, particularly urinary incontinence. Preferably, the compounds according to the invention are suitable for non-therapeutic use in people whose BMI (body mass index), defined as their body weight in kilograms divided by their height (in meters) squared, is below a level of 30, particularly below 25.

The Examples that follow are intended to illustrate the invention:

Preliminary Remarks

As a rule, IR, ¹H-NMR and/or mass spectra have been obtained for the compounds prepared. Unless otherwise stated the R_{f} values were determined using ready-made silica gel 60 TLC plates F₂₅₄ (E. Merck, Darmstadt, Item No. 1.05714) without chamber saturation. The R_{f} values obtained under the heading Alox were determined using ready-made aluminum oxide 60 TLC plates F₂₅₄ (E. Merck, Darmstadt, Item No. 1.05713) without chamber saturation. The ratios specified for the eluants are based on units by volume of the solvents in question. The units by volume specified in the case of NH₃ relate to a concentrated solution of NH₃ in water. Unless otherwise stated the acid, base and salt solutions used for working up the reaction solutions are aqueous systems of the concentrations specified.

In the asymmetric dihydroxylations, the “AD-Mix Alpha” (Item No. 39,275-8) and “AD-Mix-Beta” (Item No. 39,276-6) sold by Messrs Aldrich are used.

For chromatographic purification, silica gel made by Messrs Millipore (MATREX™, 35-70 my) is used. For chromatographic purification, Alox (E. Merck, Darmstadt, standardized aluminum oxide 90, 63-200 μm, Item No. 1.01097.9050) is used.

The HPLC data specified were measured under the parameters indicated below:

Analytical columns: Zorbax column (Agilent Technologies), SB (Stable Bond)—C18; 3.5 μm; 4.6×75 mm; column temperature: 30° C.; flow: 0.8 mL/min; injection volume: 5 μL; detection at 254 nm (methods A, B, and C).

Method A:

| | | percent by volume |
|---|---|---|
| | percent by volume of | of acetonitrile |
| | water (with 0.1% | (with 0.1% formic |
| time (min) | formic acid) | acid) |
| 0 | 95 | 5 |
| 9 | 10 | 90 |
| 10 | 10 | 90 |
| 11 | 90 | 10 |

Method B:

| | | percent by volume |
|---|---|---|
| | percent by volume | of acetonitrile |
| | of water (with 0.1% | (with 0.1% formic |
| time (min) | formic acid) | acid) |
| 0 | 95 | 5 |
| 4 | 10 | 90 |
| 10 | 10 | 90 |
| 11 | 90 | 10 |

Method C:

| | | percent by volume |
|---|---|---|
| | percent by volume | of acetonitrile |
| | of water (with 0.1% | (with 0.1% formic |
| time (min) | formic acid) | acid) |
| 0 | 95 | 5 |
| 8 | 50 | 50 |
| 9 | 10 | 90 |
| 10 | 10 | 90 |
| 11 | 90 | 10 |

Preparative column: Zorbax-Saule (Agilent Technologies), SB (Stable Bond)—C18; 3.5 μm; 30×100 mm; column temperature: ambient temperature; flow: 30 mL/min; detection at 254 nm. In preparative HPLC purification, as a rule the same gradients are used which were used when obtaining the analytical HPLC data.

The products are collected under mass control, the fractions containing the product are combined and freeze-dried.

Temperatures are given in degrees Celsius (° C.); times are generally given in minutes (min), hours (h), or days (d). If there is no specific information as to the configuration, it is not clear whether there are pure enantiomers or whether partial or even total racemization has taken place.

The following abbreviations are used above and hereinafter:
CDI carbonyldiimidazole
cyc cyclohexane
DCM dichloromethane
DIPE diisopropylether
DMF dimethylformamide
DMSO dimethylsulfoxide
dppf 1,1′-bis(diphenylphosphino)ferrocene
EtOAc ethyl acetate
EtOH ethanol
MeOH methanol
MTBE methyl-tert-butylether
PE petroleum ether
RT ambient (room) temperature (approx. 20° C.)
TBAF tetrabutylammonium fluoride trihydrate
THF tetrahydrofaran
→* denotes the binding site of a group

### amine A1: (3S,4R)-4-trifluoromethylpiperidin-3,4-diol

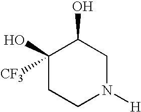

### A1a. 1-benzyl-4-trifluoromethylpyridinium chloride

A solution of 7.59 mL (65.94 mmol) of benzyl chloride in 10 mL of acetonitrile is added to a solution of 10.0 g (65.94 mmol) of 4-trifluoromethylpyridine in 40 mL of acetonitrile and the mixture is stirred for 2 hours at 80° C. A further 1.5 mL of benzyl chloride is added and the mixture is stirred for 22 hours at 80° C. The reaction mixture is cooled to RT and combined with MTBE. The precipitate is filtered off, washed with MTBE, dried in vacuo, and stored in the desiccator. Yield: 14.48 g (80% of theoretical); C₁₃H₁₁F₃N*Cl (M=273.681); calc.: molpeak (M+H)⁺: 238; found: molpeak (M+H)⁺: 238

### A1b. 1-benzyl-4-trifluoromethyl-1,2,3,6-tetrahydropyridine

3.0 g (79.36 mmol) of NaBH₄ is added batchwise with intensive cooling at 0° C. to a solution of 14.48 g (52.91 mmol) of 1-benzyl-4-trifluoromethylpyridinium chloride in 100 mL of EtOH, then the cooling is removed and the reaction mixture is stirred for 1.5 hours at 14° C. While cooling, 50 mL of water and then 50 mL of EtOH are added within 30 minutes. The reaction mixture is stirred for a further 30 minutes, the suspension formed is filtered, and the filtrate is evaporated down in vacuo. Yield: 11.72 g (92% of theoretical); C₁₃H₁₄F₃N (M=241.252); calc.: molpeak (M+H)⁺: 242; found: molpeak (M+H)⁺: 242; HPLC-MS: 3.60 min (method B).

### A1c. (3S,4R)-1-benzyl-4-trifluoromethylpiperidin-3,4-diol

43.80 g of AD-Mix-Beta is placed in 3 L of tert-butanol/water (1:1) and stirred for 20 minutes at RT. The mixture is cooled to 0° C., 2.97 g (31.25 mmol) of methanesulfonamide and 7.54 g (31.25 mmol) of 1-benzyl-4-trifluoromethyl-1,2,3,6-tetrahydropyridine are added, the cooling bath is removed, and the mixture is stirred for 8 days at RT. A further 22 g of AD-Mix-Beta and 1.5 g of methanesulfonamide are added and the mixture is again stirred for 7 days at RT. 11.2 g of sodium sulfite is added and the mixture is stirred for 1 hour. 200 mL of semisaturated sodium bicarbonate solution is added and the aqueous phase is exhaustively extracted with DCM. The combined organic phases are dried over sodium sulfate (Na₂SO₄) and evaporated down in vacuo. The crude product is purified by MPLC-MS (Grom-Sil 120 ODS 4, 10 μm, gradient 0.15% formic acid in water/acetonitrile 90:10→10:90 in 10 minutes). The eluates are combined, evaporated down in vacuo and neutralized with 100 mL of semisaturated sodium bicarbonate solution. The aqueous phase is extracted with EtOAc, and the combined organic phases are dried over sodium sulfate and evaporated down in vacuo. Yield: 1.51 g (17% of theoretical); C₁₃H₁₆F₃NO₂ (M=275.267); calc.: molpeak (M+H)⁺: 276; found: molpeak (M+H)⁺: 276; R_{f} value: 0.40 (silica gel, cyc/EtOAc 2:1).

### A1d. (3S,4R)-4-trifluoromethylpiperidin-3,4-diol

A mixture of 1.50 g (5.45 mmol) of (3S,4R)-1-benzyl-4-trifluoromethylpiperidin-3,4-diol and 170 mg of Pd/C (10%) in 17 mL of MeOH is hydrogenated at RT and 3 bar hydrogen pressure for 5 hours. The catalyst is filtered off and the filtrate is evaporated down in vacuo. Yield: 910 mg (90% of theoretical); C₆H₁₀F₃NO₂ (M=185.144); calc.: molpeak (M+H)⁺: 186; found: molpeak (M+H)⁺: 186; R_{f} value: 0.35 (silica gel, EtOAc/MeOH/NH₃ 7:3:0.3).

### amine A2: (3R,4S)-4-trifluoromethylpiperidin-3,4-diol

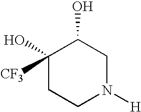

### A2a. (3R,4S)-1-benzyl-4-trifluoromethylpiperidin-3,4-diol

The product is obtained analogously to A1c starting from intermediate product A1b and AD-Mix-Alpha. The crude product is purified by HPLC-MS. Yield: 1.09 g (16% of theoretical); C₁₃H₁₆F₃NO₂ (M=275.267); calc.: molpeak (M+H)⁺: 276; found: molpeak (M+H)⁺: 276; HPLC-MS: 3.70 min (method A).

### A2b. (3R,4S)-4-trifluoromethylpiperidin-3,4-diol

The product is obtained analogously to A1d starting from (3R,4S)-1-benzyl-4-trifluoromethylpiperidin-3,4-diol. Yield: 665 mg (91% of theoretical); C₆H₁₀F₃NO₂ (M=185.144); calc.: molpeak (M+H)⁺: 186; found: molpeak (M+H)⁺: 186; R_{f} value: 0.35 (silica gel, EtOAc/MeOH/NH₃ 7:3:0.3).

### amine A3: (3R,4S)-4-methylpiperidin-3,4-diol

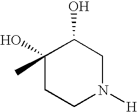

### A3a. 1-benzyl-4-methyl-1,2,3,6-tetrahydropyridine

The product is obtained analogously to A1b starting from 10.0 g (45.5 mmol) of 1-benzyl-4-methylpyridinium chloride. Yield: 7.15 g (84% of theoretical); C₁₃H₁₇N (M=187.281); calc.: molpeak (M+H)⁺: 188; found: molpeak (M+H)⁺: 188; R_{f} value: 0.95 (silica gel, EtOAc/MeOH/NH₃ 9:1:0.1).

### A3b. (3R,4S)-1-benzyl-4-methylpiperidin-3,4-diol

Under a nitrogen atmosphere 14 g AD-Mix-Alpha are placed in 50 mL water and 50 mL of tert-butanol and the mixture is stirred for 20 minutes at RT. Subsequently it is cooled to 0° C., 0.95 g (10.0 mmol) of methanesulfonic acid amide and 1.87 g (10.0 mmol) of 1-benzyl-4-methyl-1,2,3,6-tetrahydropyridine are added, the cooling bath is removed, and the reaction mixture is stirred for 24 hours at RT. 3.5 g of sodium sulfite is added to the reaction mixture and it is stirred for 1 hours. 200 mL of DCM and 200 mL of saturated sodium bicarbonate solution are added, and the organic phase is separated off and extracted with 100 mL of KHSO₄ solution. The aqueous phase is made alkaline with saturated potassium carbonate solution, extracted with 200 mL of EtOAc, and the organic phase is dried over sodium sulfate. After the desiccant and solvent have been eliminated, the crude product is purified by chromatography (silica gel, EtOAc/MeOH/NH₃ 19:1:0.1). Yield: 1.23 g (56% of theoretical); C₁₃H₁₉NO₂ (M=221.296); calc.: molpeak (M+H)⁺: 222; found: molpeak (M+H)⁺: 222; R_{f} value: 0.56 (silica gel, EtOAc/MeOH/NH₃ 19:1:0.1).

### A3c. (3R,4S)-4-methylpiperidin-3,4-diol

The product is obtained analogously to A1d starting from 1.23 g (5.57 mmol) of (3R,4S)-1-benzyl-4-methylpiperidin-3,4-diol. Yield: 730 mg (quant. yield); C₆H₁₃NO₂ (M=131.173); calc.: molpeak (M+H)⁺: 132; found: molpeak (M+H)⁺: 132; HPLC-MS: 0.93 min (method C).

### amine A4: (3S,4R)-4-methylpiperidin-3,4-diol

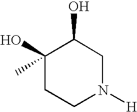

### A4a. (3S,4R)-1-benzyl-4-methylpiperidin-3,4-diol

The product is obtained analogously to A3b starting from 5.0 g (26.7 mmol) of 1-benzyl-4-methyl-1,2,3,6-tetrahydropyridine and AD-Mix-Beta. Yield: 4.68 g (79% of theoretical); C₁₃H₁₉NO₂ (M=221.296); calc.: molpeak (M+H)⁺: 222; found: molpeak (M+H)⁺: 222; R_{f} value: 0.54 (silica gel, EtOAc/MeOH/NH₃ 9:1:0.1).

### A4b. (3S,4R)-4-methylpiperidin-3,4-diol

The product is obtained analogously to A1d starting from 4.68 g (21.14 mmol) of (3S,4R)-1-benzyl-4-methylpiperidin-3,4-diol. Yield: 2.33 g (84% of theoretical); C₆H₁₃NO₂ (M=131.173); calc.: molpeak (M+H)⁺: 132; found: molpeak (M+H)⁺: 132; R_{f} value: 0.05 (silica gel, EtOAc/MeOH/NH₃ 9:1:0.1).

### amine A5: (3S,4R)-4-ethylpiperidin-3,4-diol

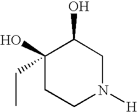

### A5a. 1-benzyl-4-ethylpyridinium chloride

The product is obtained analogously to A1a starting from 100 mL (933 mmol) of 4-ethylpyridine. Yield: 143 g (66% of theoretical); C₁₄H₁₆N*Cl (M=233.736); calc.: molpeak (M+H)⁺: 198; found: molpeak (M+H)⁺: 198; R_{f} value: 0.12 (silica gel, EtOAc/MeOH/NH₃ 9:1:0.1).

### A5b. 1-benzyl-4-ethyl-1,2,3,6-tetrahydropyridine

The product is obtained analogously to A1b starting from 143 g (614 mmol) of 1-benzyl-4-ethylpyridinium chloride. Yield: 99 g (80% of theoretical); C₁₄H₁₉N (M=201.307); calc.: molpeak (M+H)⁺: 202; found: molpeak (M+H)⁺: 202; R_{f} value: 0.91 (silica gel, EtOAc/MeOH/NH₃ 9:1:0.1).

### A5c. (3S,4R)-1-benzyl-4-ethylpiperidin-3,4-diol

The product is obtained analogously to A3b starting from 14.37 g (71.4 mmol) of 1-benzyl-4-ethyl-1,2,3,6-tetrahydropyridine and AD-Mix-Beta. Yield: 11.46 g (68% of theoretical); C₁₄H₂₁NO₂ (M=235.322); calc.: molpeak (M+H)⁺: 236; found: molpeak (M+H)⁺: 236; R_{f} value: 0.58 (silica gel, EtOAc/MeOH/NH₃ 95:5:0.5).

### A5d. (3S,4R)-4-ethylpiperidin-3,4-diol

The product may be obtained analogously to A1d starting from (3S,4R)-1-benzyl-4-ethylpiperidin-3,4-diol.

The enantiomer (3R,4S)-4-ethylpiperidin-3,4-diol may be obtained analogously to the sequence described.

### amine A6: cis-pyrrolidine-3,4-diol

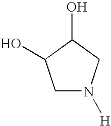

### A6a. 1-benzyl-2,5-dihydro-1H-pyrrole

1-4.6 mL (127 mmol) of benzyl chloride is added dropwise to a solution of 10 mL (127 mmol) of 2,5-dihydro-1H-pyrrole in 100 mL of acetonitrile, during which time the reaction mixture heats up to 45° C. After 1.5 hours, 300 mL of MTBE is added to the suspension, the precipitate is filtered off, and the filtrate is evaporated down. The residue is purified by chromatography (silica gel, EtOAc/MeOH/NH₃ 19:1:0.1). Yield: 4.0 g (20% of theoretical); C₁₁H₁₃N (M=159.228); calc.: molpeak (M+H)⁺: 160; found: molpeak (M+H)⁺: 160; R_{f} value: 0.60 (silica gel, EtOAc/MeOH/NH₃ 19:1:0.1).

### A6b. cis-1-benzylpyrrolidine-3,4-diol

The product is obtained analogously to A3b starting from 4.0 g (25.1 mmol) of 1-benzyl-2,5-dihydro-1H-pyrrole and AD-Mix-Beta. Yield: 0.97 g (20% of theoretical); C₁₁H₁₅NO₂ (M=193.242); calc.: molpeak (M+H)⁺: 194; found: molpeak (M+H)⁺: 194; R_{f} value: 0.15 (silica gel, EtOAc/MeOH/NH₃ 9:1:0.1).

### A6c. cis-pyrrolidine-3,4-diol

The product is obtained analogously to A1d starting from 0.97 g (5.04 mmol) of cis-1-benzylpyrrolidine-3,4-diol. Yield: 0.52 g (quant. yield); C₄H₉NO₂ (M=103.120); calc.: molpeak (M+H)⁺: 104; found: molpeak (M+H)⁺: 104; R_{f} value: 0.05 (silica gel, EtOAc/MeOH/NH₃ 5:5:0.5).

### amine A7: 4-hydroxymethylpiperidin-4-ol

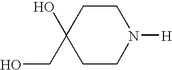

### A7a. 1-benzyl-4-hydroxymethylpiperidin-4-ol

The product is obtained analogously to A3b starting from 3.15 g (16.83 mmol) of 1-benzyl-4-methylenepiperidine and AD-Mix-Alpha. Yield: 2.92 g (79% of theoretical); C₁₃H₁₉NO₂ (M=221.296); calc.: molpeak (M+H)⁺: 222; found: molpeak (M+H)⁺: 222; R_{f} value: 0.12 (silica gel, EtOAc/MeOH/NH₃ 9:1:0.1).

### A7b. 4-hydroxymethylpiperidin-4-ol

The product is obtained analogously to A1d starting from 2.92 g (13.21 mmol) of 1-benzyl-4-hydroxymethylpiperidin-4-ol. Yield: 1.88 g (quant. yield); C₆H₁₃NO₂ (M=131.173); calc.: molpeak (M+H)⁺: 132; found: molpeak (M+H)⁺: 132; R_{f} value: 0.06 (silica gel, EtOAc/MeOH/NH₃ 5:5:0.5).

### amine A8: (S)-1-pyrrolidin-2-ylcyclopropanol

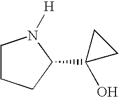

### A8a. 1-((S)-1-benzylpyrrolidin-2-yl)-cyclopropanol

First 6.91 mL (23.57 mmol) of titanium (IV) isopropoxide and then 14.3 mL (42.9 mmol, 3M in diethyl ether) of ethylmagnesium bromide are slowly added dropwise to a solution of 5.0 g (21.43 mmol) of N-benzyl-L-prolinethylester in 80 mL of dry diethyl ether cooled to −15° C. and the reaction mixture is stirred for 30 minutes at this temperature. Subsequently at approximately 10° C., 5.4 mL (42.9 mmol) of boron trifluoride-diethyl ether complex is added and the mixture is stirred for a further 75 hours at RT. While cooling, the mixture is combined with 50 mL of 1M NaOH, stirred for 1 hour at RT, combined with 100 mL of diethyl ether, and the organic phase is separated off and dried over sodium sulfate. After the desiccant and solvent have been eliminated, the residue is purified by chromatography (silica gel, EtOAc). Yield: 0.745 g (16% of theoretical); C₁₄H₁₉NO (M=217.307); calc.: molpeak (M+H)⁺: 218; found: molpeak (M+H)⁺: 218; R_{f} value: 0.17 (silica gel, EtOAc).

### A8b. (S)-1-pyrrolidin-2-ylcyclopropanol

The product is obtained analogously to A1d starting from 745 mg (3.43 mmol) of 1-((S)-1-benzylpyrrolidin-2-yl)cyclopropanol. Yield: 350 mg (80% of theoretical); C₇H₁₃NO (M=127.184); calc.: molpeak (M+H)⁺: 128; found: molpeak (M+H)⁺: 128; R_{f} value: 0.10 (silica gel, EtOAc/MeOH/NH₃ 5:5:0.5).

### intermediate product A9: 5-(4-chlorophenyl)-2-ethynyl-3-fluoropyridine

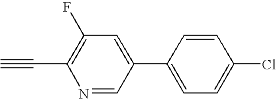

### A9a. 2,5-dibromo-3-fluoropyridine

A solution of 1.78 g (25.80 mmol) of sodium nitrite in 3.5 mL of water is added dropwise at −5° C. to a solution of 6.50 g (25.80 mmol) of 2,5-dibromopyridin-3-ylamine and 15 mL of concentrated HCl (180.62 mmol) in 15 mL of water and the mixture is stirred for 30 minutes. At 0° C., 11.41 mL (77.41 mmol) of hexafluorophosphoric acid (60% in water) is added and the mixture is stirred for 1 hour at 0° C. The diazonium salt formed is filtered off, washed with cold water, isopropanol, and diethyl ether, and dried in vacuo in the desiccator. Petroleum ether (100° C.-140° C.) is heated to 90° C., the diazonium salt is added batchwise, and the mixture is stirred until no further development of gas is detectable. The reaction mixture is cooled to RT, made alkaline with saturated sodium carbonate solution and the aqueous phase is exhaustively extracted with MTBE. The combined organic phases are washed with saturated sodium carbonate solution and water, dried over magnesium sulfate (MgSO₄), and evaporated down in vacuo. The residue is dissolved in DCM, filtered through silica gel, and the filtrate is evaporated down in vacuo. Yield: 3.30 (51% of theoretical); C₅H₂Br₂FN (M=254.883); calc.: molpeak (M+H)⁺: 253/255/257 (Br₂); found: molpeak (M+H)⁺: 253/255/257 (Br₂); R_{f} value: 0.63 (silica gel, PE/EtOAc 9:1).

### A9b. 5-bromo-2-[(tert-butyldimethylsilanyl)ethynyl]-3-fluoropyridine

Under an argon atmosphere, 2.62 mL (13.811 mmol) of tert-butylethynyldimethylsilane is added to a solution of 3.20 g (12.56 mmol) of 2,5-dibromo-3-fluoropyridine, 5.22 mL (37.67 mmol) of triethylamine, 59.8 mg (0.31 mmol) of CuI, and 220.3 mg (0.31 mmol) of bis(triphenylphosphane)palladium (II) chloride in 30 mL of absolute THF at 15° C. and the mixture is stirred for 2 hours at RT. Then a further 1 mL of tert-butylethynyldimethylsilane is added and the mixture is stirred for 1 hour at RT. The reaction mixture is evaporated down in vacuo and the residue is taken up in EtOAc. The organic phase is washed with semisaturated sodium bicarbonate solution, 5% NH₃ solution, and water, and dried over magnesium sulfate. After the desiccant and solvent have been eliminated, the residue is purified by chromatography (silica gel, PE/DCM 9:1). Yield: 1.62 g (41% of theoretical); C₁₃H₁₇BrFNSi (M=314.269); calc.: molpeak (M+H)⁺: 314/316 (Br); found: molpeak (M+H)⁺: 314/316 (Br); HPLC-MS: 7.85 min (method B).

### A9c. 2-[(tert-butyldimethylsilanyl)ethynyl]-5-(4-chlorophenyl)-3-fluoropyridine

10 mL of MeOH, 10 mL of 2N sodium carbonate solution, and 94 mg (0.13 mmol) of PdCl₂(dppf) are added to a solution of 1.61 g (5.14 mmol) of 5-bromo-2-[(tert-butyldimethylsilanyl)ethynyl]-3-fluoropyridine and 0.90 g (5.65 mmol) of 4-chlorophenylboric acid in 30 mL of 1,4-dioxane and the mixture is refluxed for 15 minutes. The reaction mixture is evaporated down in vacuo and diluted with EtOAc. The organic phase is washed with water and semisaturated sodium bicarbonate solution and dried over sodium sulfate. After the desiccant and solvent have been eliminated, the residue is purified by chromatography (silica gel, PE/DCM 1:1). Yield: 1.25 g (70% of theoretical); C₁₉H₂₁CIFNSi (M=345.913); calc.: molpeak (M+H)⁺: 346/348 (Cl); found: molpeak (M+H)⁺: 346/348 (Cl); HPLC-MS: 8.83 min (method B).

### A9d. 5-(4-chlorophenyl)-2-ethynyl-3-fluoropyridine

1.14 g (3.61 mmol) of TBAF is added at RT to a solution of 1.25 g (3.61 mmol) of 2-[(tert-butyldimethylsilanyl)ethynyl]-5-(4-chlorophenyl)-3-fluoropyridine in 30 mL of DCM and the mixture is stirred for 2 hours. The organic phase is washed with water and dried over sodium sulfate. After the desiccant and solvent have been eliminated, the residue is stirred with PE, and the precipitate is filtered off, washed with PE, and dried in the air. Yield: 0.72 g (86% of theoretical); C₁₃H₇ClFN (M=231.653); calc.: molpeak (M+H)⁺: 232/234 (Cl); found: molpeak (M+H)⁺: 232/234 (Cl); HPLC-MS: 5.81 min (method B).

### EXAMPLE 1.1

### ((S)-1-{6-[5-(4-chlorophenyl)pyridin-2-ylethynyl]-1-benzopyran-2-ylmethyl}pyrrolidin-2-yl)methanol

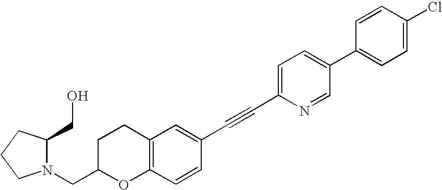

### 1.1a. (6-iodo-1-benzopyran-2-yl)-methanol

A finely triturated mixture of 4 g of silica gel (0.2-0.5 mm) and 2.02 g (5.0 mmol) of iron (III) nitrate nonahydrate is added to a solution of 1.64 g (10.00 mmol) of 1-benzopyran-2-ylmethanol and 1.40 g (5.50 mmol) of iodine in 15 mL of DCM and stirred for 12 hours at RT. A further 5 g of silica gel is added and the reaction mixture is evaporated down in vacuo. Column chromatography (silica gel, PE/EtOAc 7:3) yielded the product. Yield: 1.80 g (62% of theoretical); C₁₀H₁₁O₂ (M=290.098); calc.: molpeak (M+H)⁺: 290; found: molpeak (M+H)⁺: 290; R_{f} value: 0.45 (silica gel, PE/EtOAc 4:1).

### 1.1b. 5-bromo-2-[(tert-butyldimethylsilanyl)ethynyl]pyridine

Under an argon atmosphere, 0.80 g (4.20 mmol) of CuI and 2.90 g (4.13 mmol) of bis(triphenylphosphane)palladium (II) chloride is added to a solution of 49.90 g (201.01 mmol) of 2,5-dibromopyridine and 43.00 mL (225.57 mmol) of tert-butylethynyldimethylsilane in 500 mL of absolute THF and 120 mL of triethylamine at −7° C. and the mixture is stirred for 30 minutes at 0° C. The reaction mixture is stirred for 3.5 hours at RT, then filtered and the filtrate is evaporated down in vacuo. The residue is dissolved in 1 L of EtOAc, and the organic phase is washed with water and saturated NaCl solution, dried over sodium sulfate, and evaporated down in vacuo. The crude product is used in the next reaction step without any further purification. Yield: 59.5 g (quant. yield); C₁₃H₁₈BrNSi (M=296.278); calc.: molpeak (M+H)⁺: 296/298 (Br); found: molpeak (M+H)⁺: 296/298 (Br); R_{f} value: 0.75 (silica gel, cyc/EtOAc 8:1).

### 1.1c. 2-[(tert-butyldimethylsilanyl)ethynyl]-5-(4-chlorophenyl)pyridine

250 mL of MeOH, 220 mL of 2N sodium carbonate solution, and 1.80 g (2.46 mmol) of PdCl₂(dppf) are added to a solution of 59.5 g (201.01 mmol) of 5-bromo-2-[(tert -butyldimethylsilanyl)ethynyl]pyridine and 36.5 g (233.40 mmol) of 4-chlorophenylboric acid in 600 mL of 1,4-dioxane and the mixture is refluxed for 1 hour. The reaction mixture is evaporated down in vacuo and diluted with EtOAc. The organic phase is washed with water and semisaturated sodium bicarbonate solution, dried over sodium sulfate, and evaporated down in vacuo. The residue is purified by column chromatography (silica gel, cyc/EtOAc 9:1). Yield: 38.5 g (58% of theoretical); C₁₉H₂₂ClNSi (M=327.923); calc.: molpeak (M+H)⁺: 328/330 (Cl); found: molpeak (M+H)⁺: 328/330 (Cl); R_{f} value: 0.60 (silica gel, cyc/EtOAc 8:1).

### 1.1d. 5-(4-chlorophenyl)-2-ethynylpyridine

43.66 g (156.00 mmol) of TBAF is added to a solution of 46.50 g (142.00 mmol) of 2-[(tert-butyldimethylsilanyl)ethynyl]-5-(4-chlorophenyl)pyridine in 1 L of DCM at RT and the mixture is stirred for 2 hours. The organic phase is washed with water, dried over sodium sulfate, and evaporated down in vacuo. The residue is stirred with DIPE, and the precipitate is filtered off and washed with PE. Yield: 26.0 g (86% of theoretical); C₁₃H₈ClN (M=213.662); calc.: molpeak (M+H)⁺: 214/216 (Cl); found: molpeak (M+H)⁺: 214/216 (Cl); R_{f} value: 0.30 (silica gel, cyc/EtOAc 4:1).

### 1.1e. {6-[5-(4-chlorophenyl)pyridin-2-ylethynyl]-1-benzopyran-2-yl}methanol

Under an argon atmosphere, 48.6 mg (0.25 mmol) of CuI is added to a solution of 1.62 g (5.58 mmol) of (6-iodo-1-benzopyran-2-yl)-methanol, 1.20 g (5.60 mmol) of 5-(4-chlorophenyl)-2-ethynylpyridine, 204.1 mg (0.25 mmol) of PdCl₂(dppf), and 2.36 mL (17.00 mmol) of triethylamine in 50 mL of acetonitrile and the mixture is stirred for 6 hours at RT. The reaction mixture is poured onto water, the precipitate is filtered off and dried in the air. The residue is purified by column chromatography (silica gel, gradient DCM/EtOAc 100:0→50:50 in 30 min). Yield: 0.85 g (41% of theoretical); C₂₃H₁₈ClNO₂ (M=375.847); calc.: molpeak (M+H)⁺: 376/378 (Cl); found: molpeak (M+H)⁺: 376/378 (Cl); R_{f} value: 0.20 (silica gel, DCM/EtOAc 9:1).

### 1.1f. 6-[5-(4-chlorophenyl)pyridin-2-ylethynyl]-1-benzopyran-2-ylmethyl methanesulfonate

389 μL (5.00 mmol) of methanesulfonic acid chloride is added dropwise at 0° C. to a solution of 0.85 g (2.26 mmol) of 6-[5-(4-chlorophenyl)pyridin-2-ylethynyl]-1-benzopyran-2-yl}methanol and 403 μL (5.00 mmol) of pyridine in 20 mL of DCM and the mixture is stirred for 2 hours at RT. A further 806 μL (10.00 mmol) of methanesulfonic acid chloride is added and the mixture is stirred for 16 hours. The reaction mixture is washed with water and dilute sodium bicarbonate solution, dried over sodium sulfate, filtered through activated charcoal, and evaporated down in vacuo. The crude product is purified by column chromatography (silica gel, DCM/EtOAc 4:1). Yield: 0.70 g (68% of theoretical); C₂₄H₂₀ClNO₄S (M=453.939); calc.: molpeak (M+H)⁺: 454/456 (Cl); found: molpeak (M+H)⁺: 454/456 (Cl); R_{f} value: 0.53 (silica gel, DCM/EtOAc 9:1).

### 1.1g. ((S)-1-{6-[5-(4-chlorophenyl)pyridin-2-ylethynyl]-1-benzopyran-2-ylmethyl}pyrrolidin-2-yl)methanol

A solution of 113.5 mg (0.25 mmol) of 6-[5-(4-chlorophenyl)pyridin-2-ylethynyl]-1-benzopyran-2-ylmethyl methanesulfonate and 120 μL (1.10 mmol) of (S)-1-pyrrolidin-2-ylmethanol in 2 mL of DMF is stirred for 24 hours at 80° C. and then poured onto water. The precipitate is washed with water, a little acetone, and DIPE, and dried in the air. Yield: 80 mg (70% of theoretical); C₂₈H₂₇ClN₂O₂ (M=458.979); calc.: molpeak (M+H)⁺: 459/461 (Cl); found: molpeak (M+H)⁺: 459/461 (Cl); R_{f} value: 0.28 (silica gel, DCM/EtOAc 9:1).

The following Examples are prepared analogously starting from 6-[5-(4-chlorophenyl)pyridin-2-ylethynyl]-1-benzopyran-2-ylmethyl methanesulfonate (Example 1.1f).

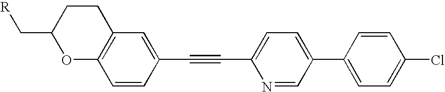

| | | | | | R_{f} value |
|---|---|---|---|---|---|
| Ex- | | | | mass | (silica gel, |
| am- | | Yield | empirical | spec- | DCM/ |
| ple | R | (%) | formula | trum | MeOH 9:1) |
| 1.2 | 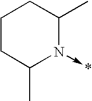 | 17 | C₃₀H₃₁ClN₂O | 471/473 [M +H]⁺ | 0.40 |
| 1.3 | 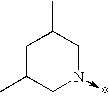 | 76 | C₃₀H₃₁ClN₂O | 471/473 [M +H]⁺ | 0.40 |

### EXAMPLE 2.1

### 6-[5-(4-chlorophenyl)pyridin-2-ylethynyl]-2-(4-methylpiperidin-1-ylmethyl)-1-benzopyran-4-one

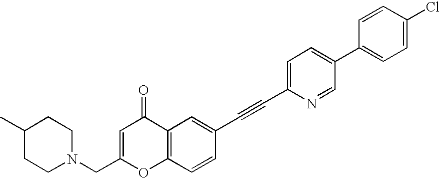

### 2.1a. 6-bromo-2-hydroxymethyl-1-benzopyran-4-on

17.03 g (105.00 mmol) of CDI is added to a solution of 26.91 g (100.00 mmol) of 6-bromo-4-oxo-4H-1-benzopyran-2-carboxylic acid in 250 mL of THF and the mixture is heated in the water bath until the development of gas has ended. This mixture is poured onto a suspension of 1.89 g (50.00 mmol) of sodium borohydride in 500 mL of water, while keeping the temperature below 10° C., and stirred for a further 2 hours at RT. The reaction mixture is poured onto 500 mL of saturated ammonium chloride solution, stirred for 1 hour at RT, and the aqueous phase is exhaustively extracted with DIPE. The combined organic phases are washed with water, dried over sodium sulfate, and evaporated down in vacuo. Yield: 19.50 g (76% of theoretical); C₁₀H₇BrO₃ (M=255.065); calc.: molpeak (M+H)⁺: 255/257 (Br); found: molpeak (M+H)⁺: 255/257 (Br); R_{f} value: 0.40 (silica gel, DCM/MeOH 9:1).

### 2.1b. 6-bromo-2-bromomethyl-1-benzopyran-4-one

A solution of 0.47 mL (5.00 mmol) of phosphorus tribromide in 2.5 mL of MTBE is added dropwise at 0° C. to a solution of 2.55 g (10.00 mmol) of 6-bromo-2-hydroxymethyl-1-benzopyran-4-one in 50 mL of MTBE and the mixture is stirred for 16 hours at RT. Ice water is added and the organic phase is separated off. The organic phase is washed with ice water, dried over magnesium sulfate, and evaporated down in vacuo. The residue is purified by column chromatography (silica gel, gradient PE/EtOAc 80:20→50:50 in 20 min). Yield: 1.69 g (53% of theoretical); C₁₀H₆Br₂O₂ (M=317.961); calc.: molpeak (M+H)⁺: 317/319/321 (2Br); found: molpeak (M+H)⁺: 317/319/321 (2Br); R_{f} value: 0.63 (silica gel, PE/EtOAc 6:4).

### 2.1c. 6-bromo-2-(4-methylpiperidin-1-ylmethyl)-1-benzopyran-4-one

A mixture of 0.60 g (1.89 mmol) of 6-bromo-2-bromomethyl-l-benzopyran-4-one, 223 μL (1.89 mmol) of 4-methylpiperidine, and 525 mg (3.80 mmol) of potassium carbonate in 50 mL of acetonitrile is stirred for 3 hours at RT and then evaporated down in vacuo. The residue is taken up in MTBE, the organic phase is washed with water, dried over magnesium sulfate, filtered through activated charcoal, and evaporated down in vacuo. Yield: 450 mg (71% of theoretical); C₁₆H₁₈BrNO₂ (M=336.224); calc.: molpeak (M+H)⁺: 336/338 (Br); found: molpeak (M+H)⁺: 336/338 (Br); HPLC-MS: 3.90 min (method B).

### 2.1d. 6-iodo-2-(4-methylpiperidin-1-ylmethyl)-1-benzopyran-4-one

Under an argon atmosphere, a mixture of 450 mg (1.34 mmol) of 6-bromo-2-(4-methylpiperidin-1-ylmethyl)-1-benzopyran-4-one, 26 mg (0.134 mmol) of CuI, 400 mg (2.68 mmol) of NaI, and 28 μL (0.268 mmol) of N,N′-dimethylethan-1,2-diamine in 1.4 mL of 1,4-dioxane is heated to 110° C. for 20 hours. The reaction mixture is combined with dilute sodium bicarbonate solution and the aqueous phase is exhaustively extracted with MTBE. The combined organic phases are dried over magnesium sulfate and evaporated down in vacuo. The residue is purified by HPLC-MS (Zorbax Bonus C18 amide-phase 5 μm, gradient 0.15% formic acid in water/acetonitrile 10/90→90/10 v/v). The eluates are evaporated down in vacuo, combined with 5% sodium carbonate solution and the aqueous phase is exhaustively extracted with MTBE. The combined organic phases are dried over magnesium sulfate and dried in vacuo. Yield: 220 mg (43% of theoretical); C₁₆H₁₈INO₂ (M=383.224); calc.: molpeak (M+H)⁺: 384; found: molpeak (M+H)⁺: 384; HPLC-MS: 4.0 min (method B).

### 2.1e. 6-[5-(4-chlorophenyl)pyridin-2-ylethynyl]-2-(4-methylpiperidin-1-ylmethyl)-1-benzopyran-4-one

The product is obtained analogously to Example 1.1e starting from 6-iodo-2-(4-methylpiperidin-1-ylmethyl)-1-benzopyran-4-one and 5-(4-chlorophenyl)-2-ethynylpyridine (Example 1.1d). The crude product is purified by HPLC-MS (Zorbax Bonus C18 amide-phase 5 μm, gradient 0.15% formic acid in water/acetonitrile 10/90→90/10 v/v). Yield: 20.5 mg (22% of theoretical); C₂₉H₂₅ClN₂O₂ (M=468.974); calc.: molpeak (M+H)⁺: 469/471 (Cl); found: molpeak (M+H)⁺: 469/471 (Cl); HPLC-MS: 5.1 min (method B).

The following compounds are obtained analogously to 2.1c, 2.1d, and 2.1e.

| | | | | retention time |
|---|---|---|---|---|
| | | yield in | mass | HPLC |
| Ex. | structure | % | spectrum | (method) |
| 2.2 | 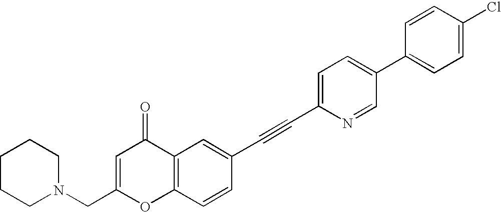 | | | |
| 2.3 | 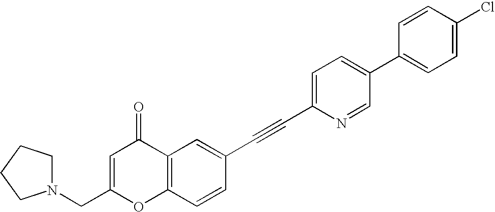 | | | |
| 2.4 | 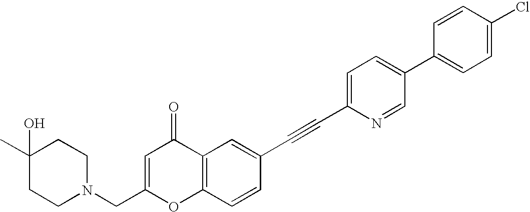 | 3 | 485/487 [M + H]⁺ | 4.39 min (B) |
| 2.5 | 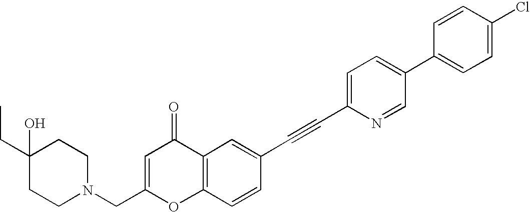 | 4 | 499/501 [M + H]⁺ | 4.52 min (B) |
| 2.6 | 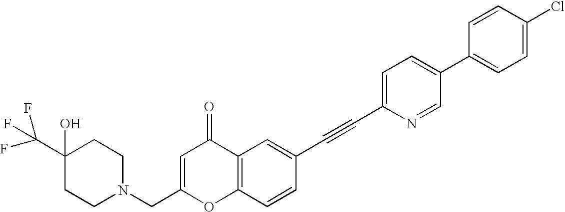 | | | |
| 2.7 | 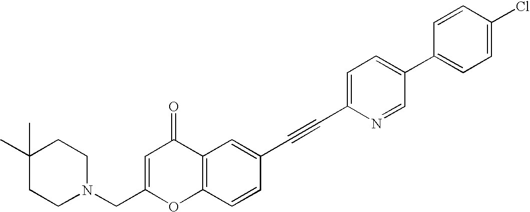 | | | |
| 2.8 | 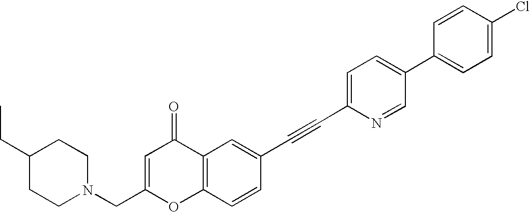 | | | |
| 2.9 | 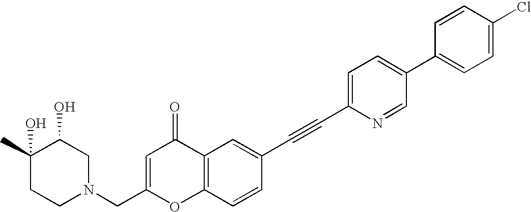 | | | |
| 2.10 | 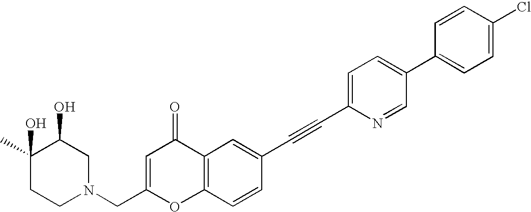 | | | |
| 2.11 | 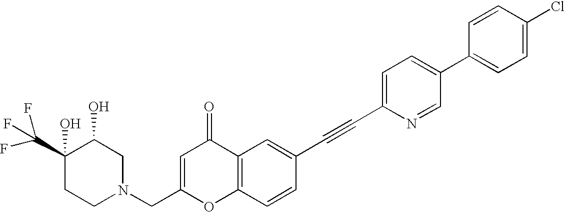 | | | |
| 2.12 | 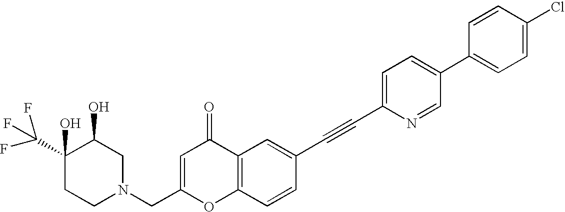 | | | |
| 2.13 | 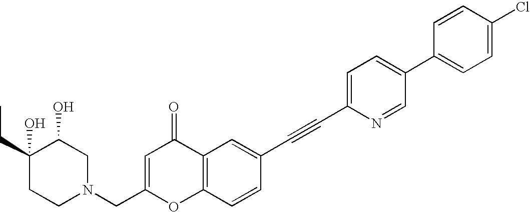 | | | |
| 2.14 | 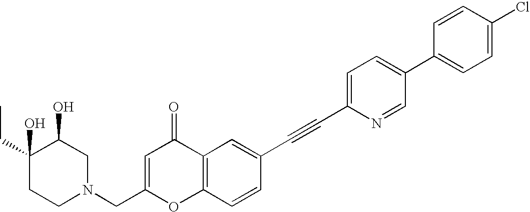 | | | |
| 2.15 | 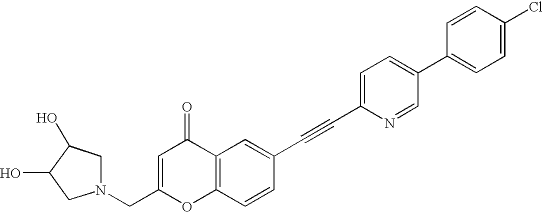 | | | |
| 2.16 | 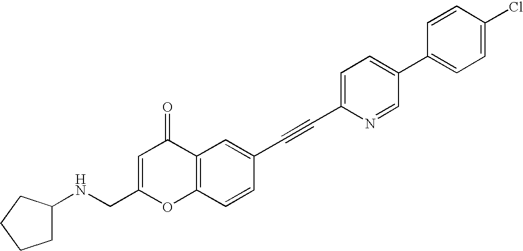 | | | |
| 2.17 | 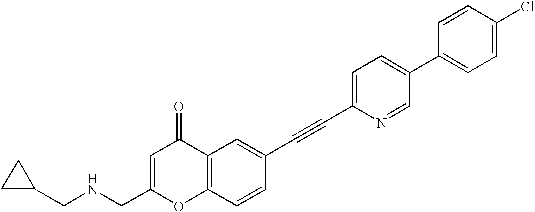 | | | |
| 2.18 | 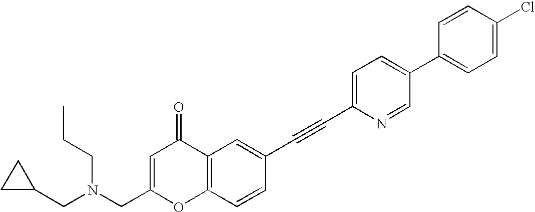 | | | |
| 2.19 | 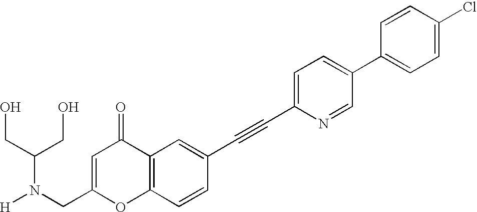 | | | |
| 2.20 | 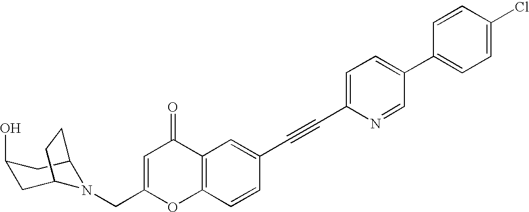 | | | |
| 2.21 | 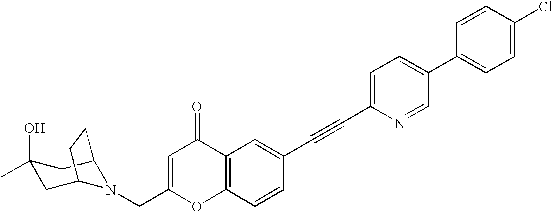 | | | |
| 2.22 | 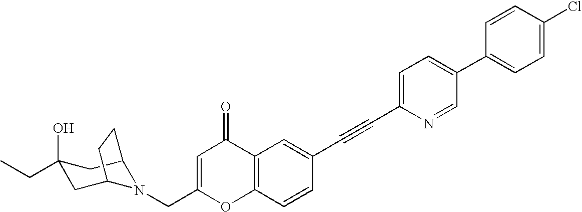 | | | |
| 2.23 | 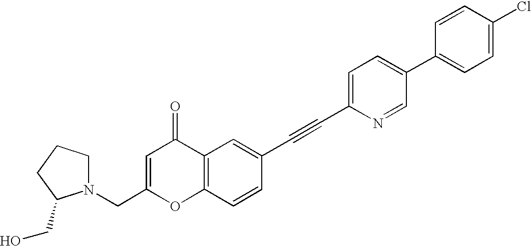 | | | |
| 2.24 | 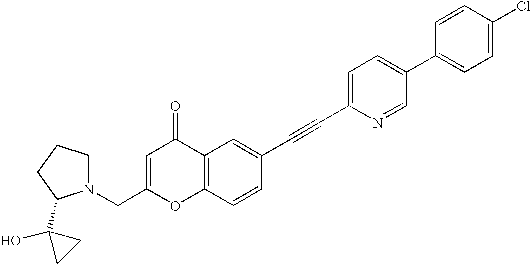 | | | |
| 2.25 | 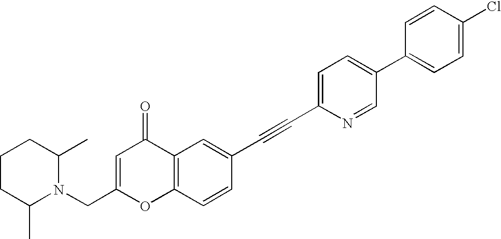 | 21 | 483/485 [M + H]⁺ | 4.8 min (B) |
| 2.26 | 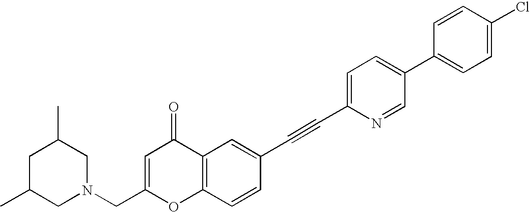 | | | |
| 2.27 | 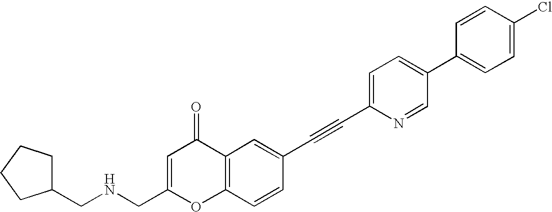 | | | |
| 2.28 | 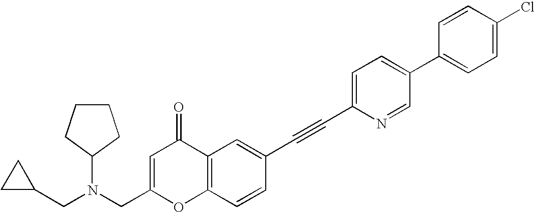 | | | |
| 2.29 | 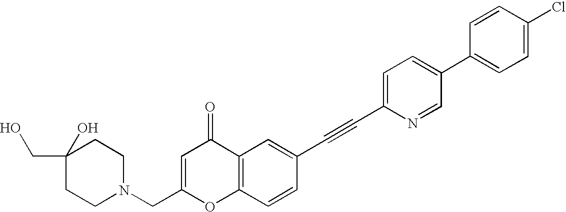 | | | |

The following compounds are obtained analogously to 2.1c, 2.1d, and 2.1e (with compound A9 as educt):

| Example | Structure |
|---|---|
| 3.1 | 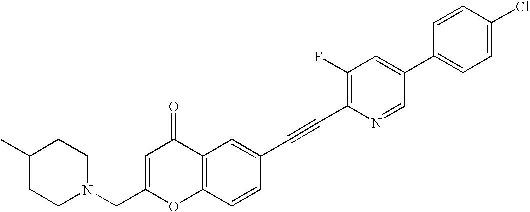 |
| 3.2 | 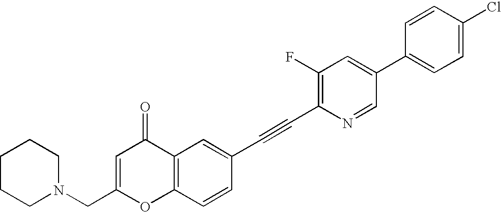 |
| 3.3 | 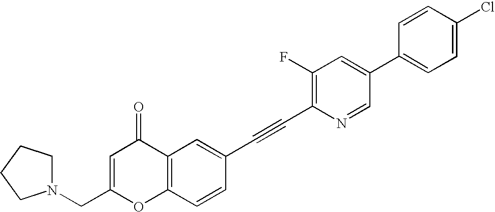 |
| 3.4 | 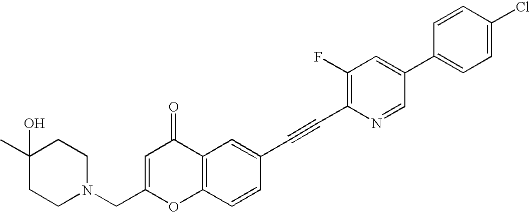 |
| 3.5 | 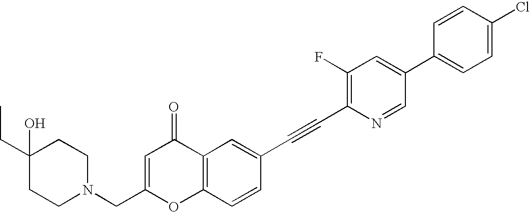 |
| 3.6 | 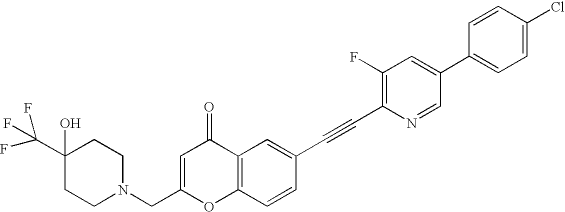 |
| 3.7 | 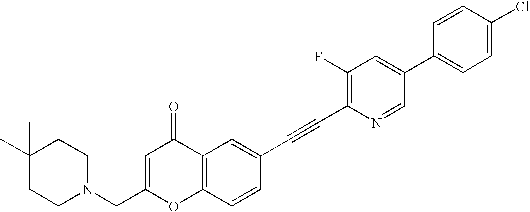 |
| 3.8 | 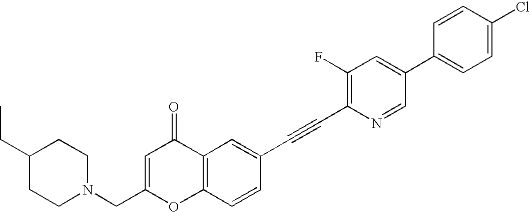 |
| 3.9 | 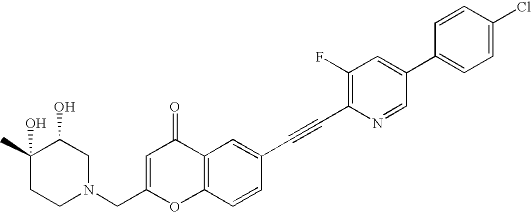 |
| 3.10 | 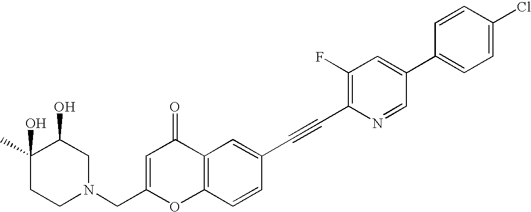 |
| 3.11 | 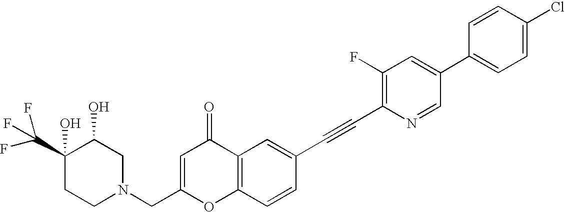 |
| 3.12 | 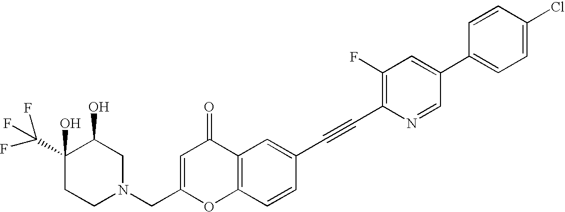 |
| 3.13 | 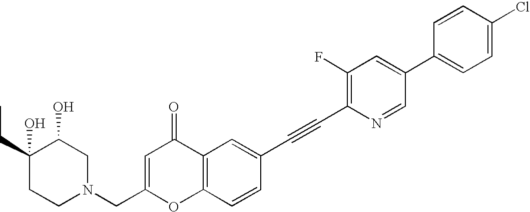 |
| 3.14 | 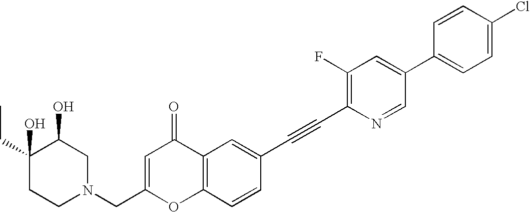 |
| 3.15 | 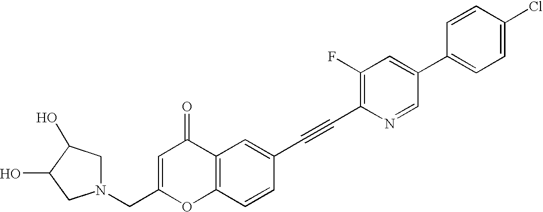 |
| 3.16 | 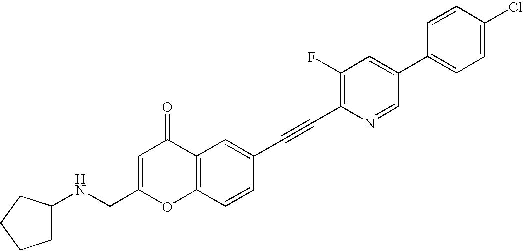 |
| 3.17 | 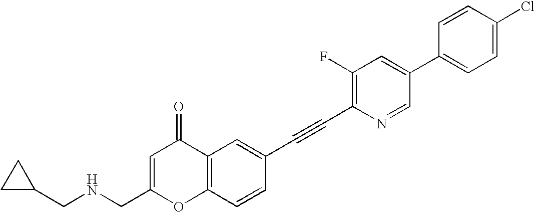 |
| 3.18 | 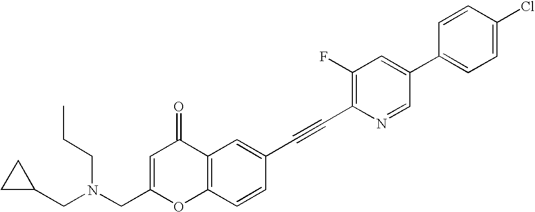 |
| 3.19 | 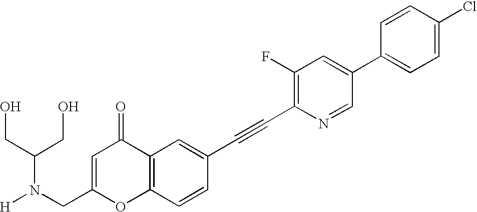 |
| 3.20 | 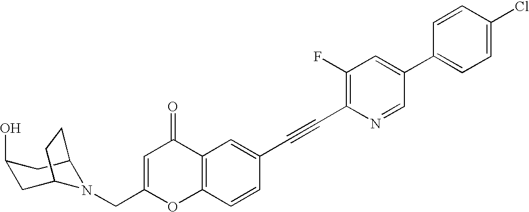 |
| 3.21 | 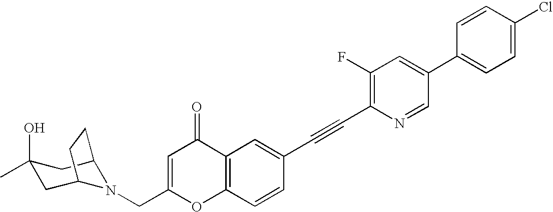 |
| 3.22 | 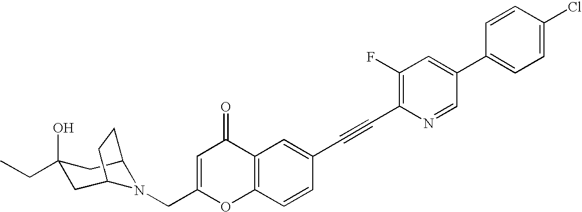 |
| 3.23 | 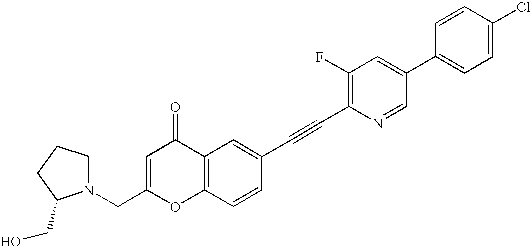 |
| 3.24 | 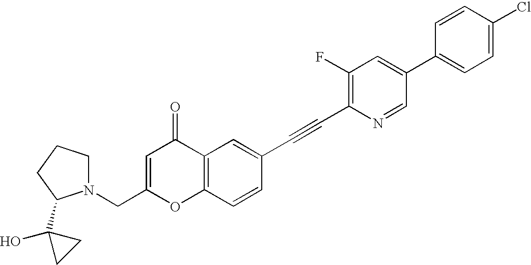 |
| 3.25 | 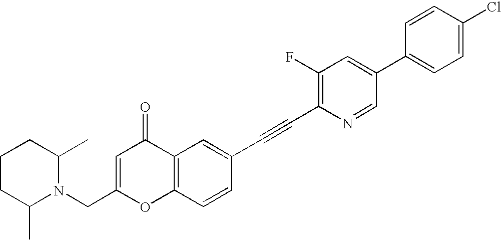 |
| 3.26 | 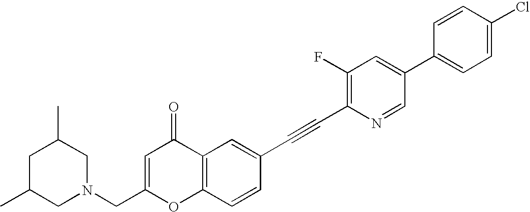 |
| 3.27 | 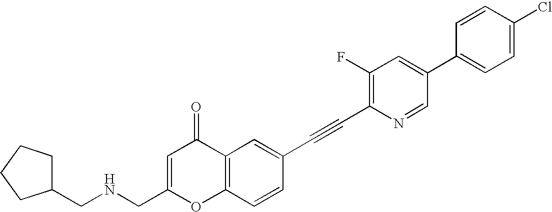 |
| 3.28 | 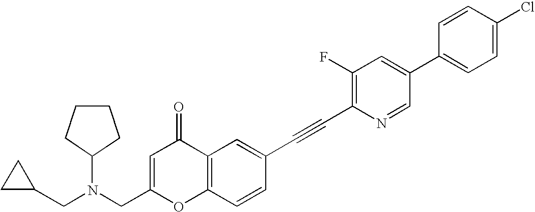 |
| 3.29 | 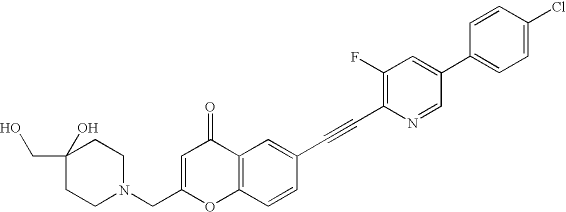 |

Some test methods for determining an MCH-receptor antagonistic activity will now be described. In addition, other test methods known to the skilled man may be used, e.g., by inhibiting the MCH-receptor-mediated inhibition of cAMP production, as described in M. Hoogduijn et al., Melanin-concentrating hormone and its receptor are expressed and functional in human skin, Biochem. Biophys. Res Commun. 296 (2002) pp. 698-701, and by biosensory measurement of the binding of MCH to the MCH receptor in the presence of antagonistic substances by plasmon resonance, as described in O. P. Karisson and S. Lofas, Flow-Mediated On-Surface Reconstitution of G-Protein Coupled Receptors for Applications in Surface Plasmon Resonance Biosensors, Anal. Biochem. 300 (2002), pp. 132-138. Other methods of testing antagonistic activity to MCH receptors are contained in the references and patent documents mentioned hereinbefore, and the description of the test methods used is hereby incorporated in this application.

MCH-1 Receptor Binding Test
Method: MCH binding to hMCH-1R transfected cells
Species: Human
Test cell: hMCH-1R stably transfected into CHO/Galpha16 cells
Results: IC₅₀ values
Membranes from CHO/Galpha16 cells stably transfected with human hMCH-1R are resuspended using a syringe (needle 0.6×25 mm) and diluted in test buffer (50 mM HEPES, 10 mM MgCl₂, 2 mM EGTA, pH 7.00; 0.1% bovine serum albumin (protease-free), 0.021% bacitracin, 1 μg/mL aprotinin, 1 μg/mL leupeptin and 1 μM phosphoramidone) to a concentration of 5 to 15 μg/mL. 200 μL of this membrane fraction (contains 1 to 3 μg of protein) are incubated for 60 minutes at ambient temperature with 100 pM of ¹²⁵I-tyrosyl melanin concentrating hormone (¹²⁵I-MCH commercially obtainable from NEN) and increasing concentrations of the test compound in a final volume of 250 μL. After the incubation the reaction is filtered using a cell harvester through 0.5% PEI treated fiberglass filters (GF/B, Unifilter Packard). The membrane-bound radioactivity retained on the filter is then determined after the addition of scintillator substance (Packard Microscint 20) in a measuring device (TopCount of Packard). The non-specific binding is defined as bound radioactivity in the presence of 1 micromolar MCH during the incubation period. The analysis of the concentration binding curve is carried out on the assumption of one receptor binding site. Standard: Non-labeled MCH competes with labeled ¹²⁵I-MCH for the receptor binding with an IC50 value of between 0.06 and 0.15 nM. The KD value of the radioligand is 0.156 nM.

MCH-1 Receptor-Coupled Ca²⁺ Mobilization Test
Method: Calcium mobilization test with human MCH (FLIPR³⁸⁴)
Species: Human
Test cells: CHO/Galpha 16 cells stably transfected with hMCH-R1

Results: 1st measurement: % stimulation of the reference (MCH 10⁻⁶M); 2nd measurement: pKB value

| | | |
|---|---|---|
| Reagents: | HBSS (10×) | (GIBCO) |
| | HEPES buffer (1 M) | (GIBCO) |
| | Pluronic F-127 | (Molecular Probes) |
| | Fluo-4 | (Molecular Probes) |
| | Probenecid | (Sigma) |
| | MCH | (Bachem) |
| | bovine serum albumin | (Serva) |
| | (protease-free) | |
| | DMSO | (Serva) |
| | Ham's F12 | (BioWhittaker) |
| | FCS | (BioWhittaker) |
| | L-Glutamine | (GIBCO) |
| | Hygromycin B | (GIBCO) |
| | PENStrep | (BioWhittaker) |
| | Zeocin | (Invitrogen) |

Clonal CHO/Galpha16 hMCH-R1 cells are cultivated in Ham's F12 cell culture medium (with L-glutamine; BioWhittaker; Cat. No.: BE12-615F). This contains per 500 mL: 10% FCS, 1% PENStrep, 5 mL of L-glutamine (200 mM stock solution), 3 mL of hygromycin B (50 mg/mL in PBS), and 1.25 mL of zeocin (100 μg/mL stock solution). One day before the experiment the cells are plated on a 384-well microtiter plate (black-walled with a transparent base, made by Costar) in a density of 2500 cells per cavity and cultivated in the above medium overnight at 37° C., 5% CO₂, and 95% relative humidity. On the day of the experiment, the cells are incubated with cell culture medium to which 2 mM Fluo-4 and 4.6 mM Probenicid have been added, at 37° C. for 45 minutes. After charging with fluorescent dye, the cells are washed four times with Hanks buffer solution (1×HBSS, 20 mM HEPES), which has been combined with 0.07% Probenicid. The test substances are diluted in Hanks buffer solution, combined with 2.5% DMSO. The background fluorescence of non-stimulated cells is measured in the presence of substance in the 384-well microtiter plate five minutes after the last washing step in the FLIPR³⁸⁴ apparatus (Molecular Devices; excitation wavelength: 488 nm; emission wavelength: bandpass 510 to 570 nm). To stimulate the cells MCH is diluted in Hanks buffer with 0.1% BSA, pipetted into the 384-well cell culture plate 35 minutes after the last washing step and the MCH-stimulated fluorescence is then measured in the FLIPR apparatus.

Data Analysis:

1st measurement: The cellular Ca²⁺ mobilization is measured as the peak of the relative fluorescence minus the background and is expressed as the percentage of the maximum signal of the reference (MCH 10⁻⁶M). This measurement serves to identify any possible agonistic effect of a test substance.

2nd measurement: The cellular Ca²⁺ mobilization is measured as the peak of the relative fluorescence minus the background and is expressed as the percentage of the maximum signal of the reference (MCH 10⁻⁶M, signal is standardized to 100%). The EC50 values of the MCH dosage activity curve with and without test substance (defined concentration) are determined graphically by the GraphPad Prism 2.01 curve program. MCH antagonists cause the MCH stimulation curve to shift to the right in the graph plotted.

The inhibition is expressed as a pKB value:
pKB=log(EC_{50(testsubstance+MCH)}/EC_{50(MCH)}−1)−log c_{(testsubstance)}

The compounds according to the invention, including their salts, exhibit an MCH-receptor antagonistic activity in the tests mentioned above. Using the MCH-1 receptor binding test described above an antagonistic activity is obtained in a dosage range from about 10⁻¹⁰ to 10⁻⁵ M, particularly from 10⁻⁹ to 10⁻⁶ M.

The following IC₅₀ values were determined using the MCH-1 receptor binding test described above:

| Compound | | |
|---|---|---|
| according to | | |
| Example No. | Name of Substance | IC₅₀ value |
| 1.1 | ((S)-1-{6-[5-(4- | 6 nM |
| | chlorophenyl)pyridin-2- | |
| | ylethynyl]-1-benzopyran- | |
| | 2-ylmethyl}pyrrolidin- | |
| | 2-yl)-methanol | |
| 2.1 | 6-[5-(4- | 3 nM |
| | chlorophenyl)pyridin-2- | |
| | ylethynyl]-2-(4- | |
| | methylpiperidin-1- | |
| | ylmethyl)-1-benzopyran-4-one | |

Some examples of formulations will be described hereinafter, wherein the term “active substance” denotes one or more compounds according to the invention, including their salts. In the case of one of the combinations with one or more active substances described, the term “active substance” also includes the additional active substances.

### EXAMPLE A

### Capsules for Powder Inhalation Containing 1 mg Active Substance

Composition:

1 capsule for powder inhalation contains:

| | | |
|---|---|---|
| active substance | 1.0 | mg |
| lactose | 20.0 | mg |
| hard gelatin capsules | 50.0 | mg |
| | 71.0 | mg |

Preparation: The active substance is ground to the particle size required for inhalation. The ground active substance is homogeneously mixed with the lactose. The mixture is packed into hard gelatin capsules.

### EXAMPLE B

### Inhalable Solution for Resipimat® Containing 1 mg Active Substance

Composition:

1 spray contains:

| | | |
|---|---|---|
| active substance | 1.0 | mg |
| benzalkonium chloride | 0.002 | mg |
| disodium edetate | 0.0075 | mg |
| purified water | to 15.0 | μL |

Preparation: The active substance and benzalkonium chloride are dissolved in water and packed into Respimat® cartridges.

### EXAMPLE C

### Inhalable Solution for Nebulizer Containing 1 mg Active Substance

Composition:

1 vial contains:

| | | |
|---|---|---|
| active substance | 0.1 | g |
| sodium chloride | 0.18 | g |
| benzalkonium chloride | 0.002 | g |
| purified water | to 20.0 | mL |

Preparation: The active substance, sodium chloride, and benzalkonium chloride are dissolved in water.

### EXAMPLE D

### Propellant Type Metered Dose Aerosol Containing 1 mg Active Substance

Composition:

1 spray contains:

| | | |
|---|---|---|
| active substance | 1.0 | mg |
| lecithin | 0.1% | |
| propellant gas | to 50.0 | μL |

Preparation: The micronized active substance is homogeneously suspended in the mixture of lecithin and propellant gas. The suspension is transferred into a pressurised container with a metering valve.

### EXAMPLE E

### Nasal Spray Containing 1 mg Active Substance

Composition:

| | | |
|---|---|---|
| active substance | 1.0 | mg |
| sodium chloride | 0.9 | mg |
| benzalkonium chloride | 0.025 | mg |
| disodium edetate | 0.05 | mg |
| purified water | to 0.1 | mL |

Preparation: The active substance and the excipients are dissolved in water and transferred into a corresponding container.

### EXAMPLE F

### Injectable solution containing 5 mg of active substance per 5 mL

Composition:

| | | |
|---|---|---|
| active substance | 5 | mg |
| glucose | 250 | mg |
| human serum albumin | 10 | mg |
| glycofurol | 250 | mg |
| water for injections | to 5 | mL |

Preparation: Glycofurol and glucose are dissolved in water for injections (WfI); human serum albumin is added; active ingredient is dissolved with heating; made up to specified volume with WfI; transferred into ampoules under nitrogen gas.

### EXAMPLE G

### Injectable Solution C 100 mg of Active Substance per 20 mL

Composition:

| | | |
|---|---|---|
| active substance | 100 | mg |
| monopotassium dihydrogen | 12 | mg |
| phosphate = KH₂PO₄ | | |
| disodium hydrogen | 2 | mg |
| phosphate = Na₂HPO₄.2H₂O | | |
| sodium chloride | 180 | mg |
| human serum albumin | 50 | mg |
| Polysorbate 80 | 20 | mg |
| water for injections | to 20 | mL |

Preparation: Polysorbate 80, sodium chloride, monopotassium dihydrogen phosphate, and disodium hydrogen phosphate are dissolved in water for injections (WfI); human serum albumin is added; active ingredient is dissolved with heating; made up to specified volume with WfI; transferred into ampoules.

### EXAMPLE H

### Lyophilisate Containing 10 mg of Active Substance

Composition:

| | | |
|---|---|---|
| Active substance | 10 | mg |
| Mannitol | 300 | mg |
| human serum albumin | 20 | mg |

Preparation: Mannitol is dissolved in water for injections (WfI); human serum albumin is added; active ingredient is dissolved with heating; made up to specified volume with WfI; transferred into vials; freeze-dried.

Solvent for lyophilisate:

| | | |
|---|---|---|
| Polysorbate 80 = Tween 80 | 20 | mg |
| mannitol | 200 | mg |
| water for injections | to 10 | mL |

Preparation: Polysorbate 80 and mannitol are dissolved in water for injections (WfI); transferred into ampoules.

### EXAMPLE I

### Tablets Containing 20 mg of Active Substance

Composition:

| | | |
|---|---|---|
| active substance | 20 | mg |
| lactose | 120 | mg |
| maize starch | 40 | mg |
| magnesium stearate | 2 | mg |
| Povidone K 25 | 18 | mg |

Preparation: Active substance, lactose, and maize starch are homogeneously mixed; granulated with an aqueous solution of povidone; mixed with magnesium stearate; compressed in a tablet press; weight of tablet: 200 mg.

### EXAMPLE J

### Capsules Containing 20 mg Active Substance

Composition:

| | | |
|---|---|---|
| active substance | 20 | mg |
| maize starch | 80 | mg |
| highly dispersed silica | 5 | mg |
| magnesium stearate | 2.5 | mg |

Preparation: Active substance, maize starch, and silica are homogeneously mixed; mixed with magnesium stearate; the mixture is packed into size 3 hard gelatin capsules in a capsule filling machine.

### EXAMPLE K

### Suppositories Containing 50 mg of Active Substance

Composition:

| | | |
|---|---|---|
| active substance | 50 | mg |
| hard fat (Adeps solidus) | q.s. ad 1700 | mg |

Preparation: Hard fat is melted at about 38° C.; ground active substance is homogeneously dispersed in the molten hard fat; after cooling to about 35° C. it is poured into chilled moulds.

### EXAMPLE L

### Injectable Solution Containing 10 mg of Active Substance Per 1 mL

Composition:

| | | |
|---|---|---|
| active substance | 10 | mg |
| mannitol | 50 | mg |
| human serum albumin | 10 | mg |
| water for injections | to 1 | mL |

Preparation: Mannitol is dissolved in water for injections (WfI); human serum albumin is added; active ingredient is dissolved with heating; made up to specified volume with WfI; transferred into ampoules under nitrogen gas.

1. A compound of formula I 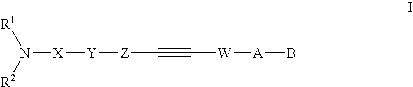
wherein:
R¹ and R² are each independently H, C₁₋₈-alkyl, C₃₋₇-cycloalkyl, or a phenyl or pyridinyl group optionally mono- or polysubstituted by identical or different groups R²⁰ and/or monosubstituted by nitro, wherein the alkyl or cycloalkyl group is optionally mono- or polysubstituted by identical or different groups R¹¹, and a —CH₂— group in position 3 or 4 of a 5-, 6-, or 7-membered cycloalkyl group is optionally replaced by —O—, —S—, or —NR¹³—, or
R¹ and R² form a C₃₋₈-alkylene bridge, wherein a —CH₂— group not adjacent to the N atom of the R¹R²N— group is optionally replaced by —CH═N—, —CH═CH—, —O—, —S—, —SO—, —(SO₂)—, —CO—, —C(═CH₂)—, or —NR¹³—, wherein in the alkylene bridge one or more H atoms are optionally replaced by identical or different groups R¹⁴, and the alkylene bridge is optionally substituted by one or two identical or different groups Cy such that the bond between the alkylene bridge and Cy is made via a single or double bond, via a common C atom forming a spirocyclic ring system, via two common adjacent C and/or N atoms forming a fused bicyclic ring system, or via three or more C and/or N atoms forming a bridged ring system;
X is a C₁₋₄-alkylene bridge, wherein if X is C₂₋₄-alkylene, one or two C atoms are optionally monosubstituted by R¹⁰, or if X is C₃₋₄-alkylene, a —CH₂—CH₂— group not immediately adjacent to the N atom of the R¹R²N— group is replaced by —CH═CH— or —C≡C—, and wherein X optionally include a substituent selected from C₂₋₆-alkenyl, C₂₋₆-alkynyl, C₃₋₇-cycloalkyl, and C₃₋₇-cycloalkyl-C₁₋₃-alkyl, as well as, independently, one, two, or three identical or different C₁₋₄-alkyl substituents, while two alkyl groups are optionally joined together forming a 3- to 7-membered cyclic group or an alkyl and an alkenyl group is optionally joined together forming a 5- to 7-membered cyclic group;
W and Z are each independently a single bond or a C₁₋₂-alkylene bridge, while two adjacent C atoms are optionally joined together by an additional C₁₋₄-alkylene bridge, and one or two C atoms are independently substituted by one or two identical or different C₁₋₃-alkyl groups, while two alkyl groups are optionally joined together to form a carbocyclic ring;
Y is Y1 or Y2 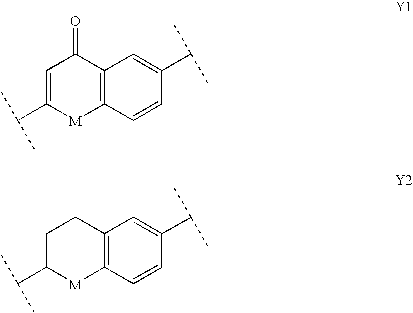
wherein the group M denotes O, S, or NR^{M}, where R^{M} is H, C₁₋₆-alkyl, C₃₋₆-alkenyl, C₃₋₆-alkynyl, C₃₋₇-cycloalkyl, or C₃₋₇-cycloalkyl-C₁₋₃-alkyl, and one or more C atoms of Y1 and Y2 are optionally independently substituted by R²⁰;
A is independently phenyl, pyridinyl, pyrimidinyl, pyrazinyl, pyridazinyl, naphthyl, tetrahydronaphthyl, indolyl, dihydroindolyl, quinolinyl, dihydroquinolinyl, tetrahydroquinolinyl, isoquinolinyl, dihydroisoquinolinyl, tetrahydroisoquinolinyl, benzimidazolyl, benzoxazolyl, thienyl, furanyl, benzothienyl, or benzofuranyl, each optionally mono- or polysubstituted at one or more C atoms by identical or different groups R²⁰, and in the case of a phenyl ring is additionally optionally monosubstituted by nitro, and one or more NH groups are optionally substituted by R²¹;
B is Y, A, C₁₋₆-alkyl, C₁₋₆-alkenyl, C₁₋₆-alkynyl, C₃₋₇-cycloalkyl, C₅₋₇-cycloalkenyl, C₃₋₇-cycloalkyl-C₁₋₃-alkyl, C₃₋₇-cycloalkenyl-C₁₋₃-alkyl, C₃₋₇-cycloalkyl-C₁₋₃-alkenyl, or C₃₋₇-cycloalkyl-C₁₋₃-alkynyl, wherein one or more C atoms are independently optionally mono- or polysubstituted by halogen and/or monosubstituted by hydroxy or cyano and/or cyclic groups are optionally mono- or polysubstituted by identical or different groups R²⁰;
Cy is a saturated 3- to 7-membered carbocyclic group, an unsaturated 4- to 7-membered carbocyclic group, a phenyl group, a saturated 4- to 7-membered or unsaturated 5- to 7-membered heterocyclic group with an N, O, or S atom as heteroatom, a saturated or unsaturated 5- to 7-membered heterocyclic group with two or more N atoms or with one or two N atoms and an O or S atom as heteroatoms, an aromatic heterocyclic 5- or 6-membered group with one or more identical or different heteroatoms selected from N, O, and/or S, wherein the saturated 6- or 7-membered groups thereof are optionally also present as bridged ring systems with an imino, (C₁₋₄-alkyl)-imino, methylene, (C₁₋₄-alkyl)-methylene, or di-(C₁₋₄-alkyl)-methylene bridge, and wherein the cyclic groups thereof are optionally mono- or polysubstituted at one or more C atoms by identical or different groups R²⁰, and in the case of a phenyl group are additionally optionally monosubstituted by nitro, and/or one or more NH groups are optionally substituted by R²¹;
R¹⁰ is hydroxy, ω-hydroxy-C₁₋₃-alkyl, C₁₋₄-alkoxy, or C₁₋₄-alkoxy-C₁₋₃-alkyl;
R¹¹ is halogen, C₁₋₆-alkyl, C₂₋₆-alkenyl, C₂₋₆-alkynyl, R¹⁵—O—, R¹⁵—O—CO—, R¹⁵—CO—O—-cyano, R¹⁶R¹⁷N, R¹⁸R¹⁹N—CO—, or Cy, wherein one or more C atoms thereof are optionally independently substituted by halogen, OH, CN, CF₃, C₁₋₃-alkyl, or hydroxy-C₁₋₃-alkyl;
R¹³ is R¹⁷;
R¹⁴ is halogen, cyano, C₁₋₆-alkyl, C₂₋₆-alkenyl, C₂₋₆-alkynyl, R¹⁵—O—R¹⁵—O—CO—, R¹⁵—CO—O—R¹⁶R¹⁷N, R¹⁸R¹⁹N—CO—,R¹⁵—O—C₁₋₃-alkyl, R¹⁵—O—CO-C₁₋₃-alkyl, R¹⁵—SO₂—NH—, R¹⁵—O—CO—NH—C₁₋₃-alkyl, R¹⁵—SO₂—NH—C₁₋₃-alkyl, R¹⁵—CO—C₁₋₃-alkyl, R¹⁵—CO—O—C₁₋₃-alkyl, R¹⁶R¹⁷N—C₁₋₃-alkyl, R¹⁸R¹⁹N—CO—C₁₋₃-alkyl, or Cy-C₁₋₃-alkyl;
R¹⁵ is H, C₁₋₄-alkyl, C₃₋₇-cycloalkyl, C₃₋₇-cycloalkyl-C₁₋₃-alkyl, phenyl, phenyl-C₁₋₃-alkyl, pyridinyl, or pyridinyl-C₁₋₃-alkyl;
R¹⁶ is H, C₁₋₆-alkyl, C₃₋₇-cycloalkyl, C₃₋₇-cycloalkyl-C₁₋₃-alkyl, C₄₋₇-cycloalkenyl, C₄₋₇-cycloalkenyl-C₁₋₃-alkyl, ω-hydroxy-C₂₋₃-alkyl, ω-(C₁₋₄-alkoxy)-C₂₋₃-alkyl, amino-C₂₋₆-alkyl, C₁₋₄-alkyl-amino-C₂₋₆-alkyl, di-(C₁₋₄-alkyl)-amino-C₂₋₆-alkyl, or cyclo-C₃₋₆-alkyleneimino-C₂₋₆-alkyl;
R¹⁷ is R¹⁶, phenyl, phenyl-C₁₋₃-alkyl, pyridinyl, C₁₋₄-alkylcarbonyl, hydroxycarbonyl-C₁₋₃-alkyl, C₁₋₄-alkoxycarbonyl, C₁₋₄-alkoxycarbonyl-C₁₋₃-alkyl, C₁₋₄-alkylcarbonylamino-C₂₋₃-alkyl, N—(C₁₋₄-alkylcarbonyl)-N—(C₁₋₄-alkyl)-amino-C₂₋₃-alkyl, C₁₋₄-alkylsulfonyl, C₁₋₄-alkylsulfonylamino-C₂₋₃-alkyl, or N—(C₁₋₄-alkylsulfonyl)-N(—C₁₋₄-alkyl)-amino-C₂₋₃-alkyl;
R¹⁸ and R¹⁹ are each independently H or C₁₋₆-alkyl;
R²⁰ is halogen, hydroxy, cyano, C₁₋₆-alkyl, C₂₋₆-alkenyl, C₂₋₆-alkynyl, C₃₋₇-cycloalkyl, C₃₋₇-cycloalkyl-C₁₋₃-alkyl, hydroxy-C₁₋₃-alkyl, R²²—C₁₋₃-alkyl, or R²²;
R²¹ is C₁₋₄-alkyl, ω-hydroxy-C₂₋₆-alkyl, ω-C₁₋₄-alkoxy-C₂₋₆-alkyl, ω-C₁₋₄-alkyl-amino-C₂₋₆-alkyl, ω-di-(C₁₋₄-alkyl)-amino-C₂₋₆-alkyl, ω-cyclo-C₃₋₆-alkyleneimino-C₂₋₆-alkyl, phenyl, phenyl-C₁₋₃-alkyl, C₁₋₄-alkyl-carbonyl, C₁₋₄-alkoxy-carbonyl, C₁₋₄-alkylsulfonyl, aminosulfonyl, C₁₋₄-alkylaminosulfonyl, di-C₁₋₄-alkylaminosulfonyl, or cyclo-C₃₋₆-alkylene-iminosulfonyl; and
R²² is pyridinyl, phenyl, phenyl-C₁₋₃-alkoxy, cyclo-C₃₋₆-alkyleneimino-C₂₋₄-alkoxy, OHC—, HO—N═HC—, C₁₋₄-alkoxy-N═HC—, C₁₋₄-alkoxy, C₁₋₄-alkylthio, carboxy, C₁₋₄-alkylcarbonyl, C₁₋₄-alkoxycarbonyl, aminocarbonyl, C₁₋₄-alkylaminocarbonyl, di-(C₁₋₄-alkyl)-aminocarbonyl, cyclo-C₃₋₆-alkyl-aminocarbonyl, cyclo-C₃₋₆-alkyleneiminocarbonyl, phenylaminocarbonyl, cyclo-C₃₋₆-alkyleneimino-C₂₋₄-alkyl-aminocarbonyl, C₁₋₄-alkyl-sulfonyl, C₁₋₄-alkyl-sulfinyl, C₁₋₄-alkyl-sulfonylamino, amino, C₁₋₄-alkylamino, di-(C₁₋₄-alkyl)-amino, C₁₋₄-alkyl-carbonylamino, cyclo-C₃₋₆-alkyleneimino, phenyl-C₁₋₃-alkylamino, N—(C₁₋₄-alkyl)-phenyl-C₁₋₃-alkylamino, acetylamino, propionylamino, phenylcarbonyl, phenylcarbonylamino, phenylcarbonylmethylamino, hydroxy-C₂₋₃-alkylaminocarbonyl, (4-morpholinyl)carbonyl, (1-pyrrolidinyl)carbonyl, (1-piperidinyl)carbonyl, (hexahydro-1-azepinyl)carbonyl, (4-methyl-1-piperazinyl)carbonyl, methylenedioxy, aminocarbonylamino, or C₁₋₄-alkylaminocarbonylamino,
wherein in each of the abovementioned groups and residues one or more C atoms are additionally optionally mono- or polysubstituted by F and/or one or two C atoms are independently optionally monosubstituted by Cl or Br and/or one or more phenyl rings independently optionally contain one, two, or three substituents selected from F, Cl, Br, I, cyano, C₁₋₄-alkyl, C₁₋₄-alkoxy, difluoromethyl, trifluoromethyl, hydroxy, amino, C₁₋₃-alkylamino, di-(C₁₋₃-alkyl)-amino, acetylamino, aminocarbonyl, difluoromethoxy, trifluoromethoxy, amino-C₁₋₃-alkyl, C₁₋₃-alkylamino-C₁₋₃-alkyl-, and di-(C₁₋₃-alkyl)-amino-C₁₋₃-alkyl and/or are optionally monosubstituted by nitro, and
the H atom of any carboxy group present or an H atom bound to an N atom are each optionally replaced by a group which can be cleaved in vivo, and
the tautomers, enantiomers, salts, and mixtures thereof,
and excluding the following compounds:
6-[5-(4-chlorophenyl)pyridin-2-ylethynyl]-2-pyrrolidin-1-ylmethyl-1,2,3,4-tetrahydroquinoline,
6-[5-(4-chlorophenyl)pyridin-2-ylethynyl]-1-methyl-2-pyrrolidin-1-ylmethyl-1,2,3,4-tetrahydroquinoline, and
5-(4-chlorophenyl)-2-[2-(4-methylpiperidin-1-ylmethyl)chroman-6-ylethynyl]pyridine.

2. The compound of formula (I) according to claim 1, wherein:
R¹ and R² are independently H, C₁₋₆-alkyl, C₃₋₅-alkenyl, C₃₋₅-alkynyl, C₃₋₇-cycloalkyl, hydroxy-C₃₋₇-cycloalkyl, C₃₋₇-cycloalkyl-C₁₋₃-alkyl, (hydroxy-C₃₋₇-cycloalkyl)-C₁₋₃-alkyl, hydroxy-C₂₋₄-alkyl, ω-NC—C₂₋₃-alkyl, C₁₋₄-alkoxy-C₂₋₄-alkyl, hydroxy-C₁₋₄-alkoxy-C₂₋₄-alkyl, C₁₋₄-alkoxy-carbonyl-C₁₋₄-alkyl, carboxyl-C₁₋₄-alkyl, amino-C₂₋₄-alkyl, C₁₋₄-alkyl-amino-C₂₋₄-alkyl, di-(C₁₋₄-alkyl)-amino-C₂₋₄-alkyl, cyclo-C₃₋₆-alkyleneimino-C₂₋₄-alkyl, pyrrolidin-3-yl, N—(C₁₋₄-alkyl)-pyrrolidin-3-yl, pyrrolidinyl-C₁₋₃-alkyl, N—(C₁₋₄-alkyl)-pyrrolidinyl-C₁₋₃-alkyl, piperidin-3-yl, piperidin-4-yl, N—(C₁₋₄-alkyl)-piperidin-3-yl, N—(C₁₋₄-alkyl)-piperidin-4-yl, piperidinyl-C₁₋₃-alkyl, N—(C₁₋₄-alkyl)-piperidinyl-C₁₋₃-alkyl, tetrahydropyran-3-yl, tetrahydropyran-4-yl, phenyl, phenyl-C₁₋₃-alkyl, pyridyl, or pyridyl-C₁₋₃-alkyl, wherein one or more C atoms thereof are optionally independently mono- or polysubstituted by F, C₁₋₃-alkyl, or hydroxy-C₁₋₃-alkyl, and/or one or two C atoms thereof are optionally independently monosubstituted by Cl, Br, OH, CF₃, or CN, and the phenyl or pyridyl group is optionally mono- or polysubstituted by identical or different groups R²⁰, and in the case of a phenyl group is additionally optionally monosubstituted by nitro.

3. The compound of formula (I) according to claim 1, wherein:
R¹ and R² together with the N atom to which they are bound is a heterocyclic group selected from pyrrolidine, piperidine, piperazine wherein the free imine function is substituted by R¹³, and morpholine, wherein one or more H atoms are optionally replaced by identical or different groups R¹⁴, and/or the heterocyclic group is optionally substituted by one or two identical or different Cy groups in such a way that the bond between the alkylene bridge and Cy is made via a single or double bond, via a common C atom forming a spirocyclic ring system, via two common adjacent C and/or N atoms forming a fused bicyclic ring system, or via three or more C and/or N atoms forming a bridged ring system.

4. The compound of formula (I) according to claim 1, wherein:
X is a methylene or ethylene bridge optionally substituted by one or two identical or different C₁₋₃-alkyl-substituents and/or a substituent selected from C₂₋₆-alkenyl, C₂₋₆-alkynyl, C₃₋₆-cycloalkyl, and C₃₋₆-cycloalkyl-C₁₋₃-alkyl, wherein two alkyl substituents are optionally joined together forming a 3- to 6-membered carbocyclic ring system.

5. The compound of formula (I) according to claim 1, wherein:
Z is a single bond or ethylene; and
W is a single bond.

6. The compound of formula (I) according to claim 1, wherein: 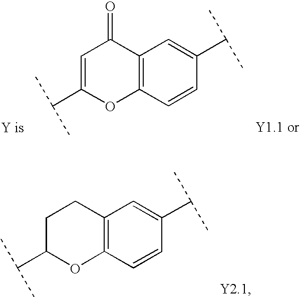
wherein one or more C atoms thereof are optionally independently substituted with R²⁰.

7. The compound of formula (I) according to claim 1, wherein:
A is phenyl, pyridinyl, pyrimidinyl, pyrazinyl, or pyridazinyl, each optionally mono- or polysubstituted at one or more C atoms by identical or different groups R²⁰, or in the case of a phenyl ring are additionally optionally monosubstituted by nitro.

8. The compound of formula (I) according to claim 1, wherein:
B is phenyl, cyclohexenyl, pyridyl, thienyl, or furanyl, each optionally mono- or polysubstituted at one or more C atoms by identical or different groups R²⁰, or in the case of a phenyl group are additionally optionally monosubstituted by nitro.

9. The compound of formula (I) according to claim 1, wherein: 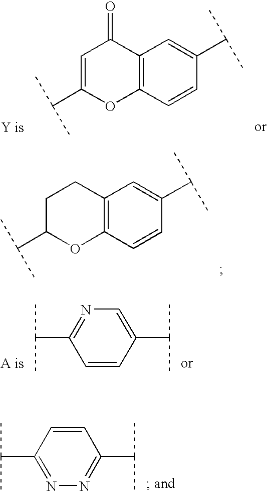
B is phenyl, cyclohexenyl, pyridyl, thienyl, or furanyl,
wherein Y and A are unsubstituted or monosubstituted by R²⁰, and B is unsubstituted or independently mono-, di-, or trisubstituted by R²⁰, or in the case of a phenyl ring are additionally optionally monosubstituted by nitro.

10. The compound of formula (I) according to claim 1, wherein:
R²⁰ is F, Cl, Br, I, OH, cyano, methyl, difluoromethyl, trifluoromethyl, ethyl, n-propyl, isopropyl, amino, acetyl, methoxy, difluoromethoxy, trifluoromethoxy, ethoxy, n-propoxy or isopropoxy.

11. A physiologically acceptable salt of the compound according to claim 1.

12. A pharmaceutical formulation comprising the compound according to claim 1 and one or more physiologically acceptable excipients or inert carriers or diluents.

13. A pharmaceutical formulation comprising the compound according to claim 2 and one or more physiologically acceptable excipients or inert carriers or diluents.

14. A pharmaceutical formulation comprising the compound according to claim 3 and one or more physiologically acceptable excipients or inert carriers or diluents.

15. A pharmaceutical formulation comprising the physiologically acceptable salt according to claim 11 and one or more physiologically acceptable excipients or inert carriers or diluents.

16. The pharmaceutical formulation according to claim 12 further comprising a second active substance selected from the group consisting of active substances for the treatment of diabetes, active substances for the treatment of diabetic complications, active substances for the treatment of obesity, active substances for the treatment of high blood pressure, active substances for the treatment of hyperlipidemia or arteriosclerosis, active substances for the treatment of arthritis, active substances for the treatment of anxiety states, and active substances for the treatment of depression.

17. The pharmaceutical formulation according to claim 13 further comprising a second active substance selected from the group consisting of active substances for the treatment of diabetes, active substances for the treatment of diabetic complications, active substances for the treatment of obesity, active substances for the treatment of high blood pressure, active substances for the treatment of hyperlipidemia or arteriosclerosis, active substances for the treatment of arthritis, active substances for the treatment of anxiety states, and active substances for the treatment of depression.

18. The pharmaceutical formulation according to claim 14 further comprising a second active substance selected from the group consisting of active substances for the treatment of diabetes, active substances for the treatment of diabetic complications, active substances for the treatment of obesity, active substances for the treatment of high blood pressure, active substances for the treatment of hyperlipidemia or arteriosclerosis, active substances for the treatment of arthritis, active substances for the treatment of anxiety states, and active substances for the treatment of depression.

19. The pharmaceutical formulation according to claim 15 further comprising a second active substance selected from the group consisting of active substances for the treatment of diabetes, active substances for the treatment of diabetic complications, active substances for the treatment of obesity, active substances for the treatment of high blood pressure, active substances for the treatment of hyperlipidemia or arteriosclerosis, active substances for the treatment of arthritis, active substances for the treatment of anxiety states, and active substances for the treatment of depression.

20. A method for influencing the eating behavior of a mammal comprising administering to the mammal an effective amount of the compound according to claim 1.

21. A method for influencing the eating behavior of a mammal comprising administering to the mammal an effective amount of the compound according to claim 2.

22. A method for influencing the eating behavior of a mammal comprising administering to the mammal an effective amount of the compound according to claim 3.

23. A method for influencing the eating behavior of a mammal comprising administering to the mammal an effective amount of the physiologically acceptable salt according to claim 11.

24. A method for reducing the body weight and/or for preventing an increase in the body weight of a mammal comprising administering to the mammal an effective amount of the compound according to claim 1.

25. A method for reducing the body weight and/or for preventing an increase in the body weight of a mammal comprising administering to the mammal an effective amount of the compound according to claim 2.

26. A method for reducing the body weight and/or for preventing an increase in the body weight of a mammal comprising administering to the mammal an effective amount of the compound according to claim 3.

27. A method for reducing the body weight and/or for preventing an increase in the body weight of a mammal comprising administering to the mammal an effective amount of the physiologically acceptable salt according to claim 11.

28. A method for preventing or treating a metabolic disorder or eating disorder in a mammal comprising administering to the mammal an effective amount of the compound according to claim 1.

29. A method for preventing or treating a metabolic disorder or eating disorder in a mammal comprising administering to the mammal an effective amount of the compound according to claim 2.

30. A method for preventing or treating a metabolic disorder or eating disorder in a mammal comprising administering to the mammal an effective amount of the compound according to claim 3.

31. A method for preventing or treating a metabolic disorder or eating disorder in a mammal comprising administering to the mammal an effective amount of the physiologically acceptable salt according to claim 11.

32. The method according to claim 28, wherein the metabolic disorder or eating disorder is obesity, bulimia, bulimia nervosa, cachexia, anorexia, anorexia nervosa, or hyperphagia.

33. The method according to claim 29, wherein the metabolic disorder or eating disorder is obesity, bulimia, bulimia nervosa, cachexia, anorexia, anorexia nervosa, or hyperphagia.

34. The method according to claim 30, wherein the metabolic disorder or eating disorder is obesity, bulimia, bulimia nervosa, cachexia, anorexia, anorexia nervosa, or hyperphagia.

35. The method according to claim 31, wherein the metabolic disorder or eating disorder is obesity, bulimia, bulimia nervosa, cachexia, anorexia, anorexia nervosa, or hyperphagia.

36. A method for preventing or treating diabetes, diabetic retinopathy, diabetic neuropathy, diabetic nephropathy, insulin resistance, pathological glucose tolerance, encephalorrhagia, cardiac insufficiency, arteriosclerosis, high blood pressure, arthritis, or gonitis in a mammal comprising administering to the mammal an effective amount of the compound according to claim 1.

37. A method for preventing or treating diabetes, diabetic retinopathy, diabetic neuropathy, diabetic nephropathy, insulin resistance, pathological glucose tolerance, encephalorrhagia, cardiac insufficiency, arteriosclerosis, high blood pressure, arthritis, or gonitis in a mammal comprising administering to the mammal an effective amount of the compound according to claim 2.

38. A method for preventing or treating diabetes, diabetic retinopathy, diabetic neuropathy, diabetic nephropathy, insulin resistance, pathological glucose tolerance, encephalorrhagia, cardiac insufficiency, arteriosclerosis, high blood pressure, arthritis, or gonitis in a mammal comprising administering to the mammal an effective amount of the compound according to claim 3.

39. A method for preventing or treating hyperlipidemia, cellulitis, fat accumulation, malignant mastocytosis, systemic mastocytosis, emotional disorders, affective disorders, depression, anxiety, sleep disorders, reproductive disorders, sexual disorders, memory disorders, epilepsy, forms of dementia, or hormonal disorders in a mammal comprising administering to the mammal an effective amount of the compound according to claim 1.

40. A method for preventing or treating hyperlipidemia, cellulitis, fat accumulation, malignant mastocytosis, systemic mastocytosis, emotional disorders, affective disorders, depression, anxiety, sleep disorders, reproductive disorders, sexual disorders, memory disorders, epilepsy, forms of dementia, or hormonal disorders in a mammal comprising administering to the mammal an effective amount of the compound according to claim 2.

41. A method for preventing or treating hyperlipidemia, cellulitis, fat accumulation, malignant mastocytosis, systemic mastocytosis, emotional disorders, affective disorders, depression, anxiety, sleep disorders, reproductive disorders, sexual disorders, memory disorders, epilepsy, forms of dementia, or hormonal disorders in a mammal comprising administering to the mammal an effective amount of the compound according to claim 3.

42. A method for preventing or treating urinary incontinence, hyperactive urinary bladder, urgency, nycturia, or enuresis in a mammal comprising administering to the mammal an effective amount of the compound according to claim 1.

43. A method for preventing or treating urinary incontinence, hyperactive urinary bladder, urgency, nycturia, or enuresis in a mammal comprising administering to the mammal an effective amount of the compound according to claim 2.

44. A method for preventing or treating urinary incontinence, hyperactive urinary bladder, urgency, nycturia, or enuresis in a mammal comprising administering to the mammal an effective amount of the compound according to claim 3.

45. A method for treating dependencies and/or withdrawal symptoms in a mammal comprising administering to the mammal an effective amount of the compound according to claim 1.

46. A method for treating dependencies and/or withdrawal symptoms in a mammal comprising administering to the mammal an effective amount of the compound according to claim 2.

47. A method for treating dependencies and/or withdrawal symptoms in a mammal comprising administering to the mammal an effective amount of the compound according to claim 3.
